(19) United States
(12) Patent Application Publication
(10) Pub. No.: US 2015/0024955 A1
(43) Pub. Date: Jan. 22, 2015

(54) Methods of Identifying Modulators of Dephosphorylation of Histone Deacetylase

(71) Applicant: THE J. DAVID GLADSTONE INSTITUTES, SAN FRANCISCO, CA (US)

(72) Inventors: Eric Verdin, Mill Valley, CA (US); Maribel Parra, San Francisco, CA (US)

(21) Appl. No.: 14/335,631

(22) Filed: Jul. 18, 2014

Related U.S. Application Data
(62) Division of application No. 11/741,561, filed on Apr. 27, 2007, now abandoned.
(60) Provisional application No. 60/795,767, filed on Apr. 27, 2006.

(51) Int. Cl. C12Q 1/42; G01N 33/50; G01N 33/573; C12Q 1/44

(52) U.S. Cl. CPC C12Q 1/42; C12Q 1/44; G01N 33/5023; G01N 33/573; G01N 2500/04; G01N 2500/02
USPC 506/9; 435/7.4; 435/6.13; 435/6.11; 435/6.12

(57) ABSTRACT

The present invention relates to screening methods that make use of a histone deacetylase interacting with a myosin phosphatase for the identification of novel therapeutics useful for inhibiting or inducing apoptosis and for the treatment of pathological conditions, such as smooth muscle cell disorder, cardiac hypertrophy or asthma. Also disclosed are methods for inhibiting or inducing apoptosis and for treatment of a pathological condition by administering to a mammal a therapeutically effective amount of a compound that inhibits or increases the dephosphorylation of a histone deacetylase by a myosin phosphatase or inhibits or increases the binding of a histone deacetylase to a myosin phosphatase.

### CROSS-REFERENCES TO RELATED APPLICATIONS

This application claims benefit of U.S. provisional application Ser. No. 60/795,767, filed Apr. 27, 2006, the disclosure of which is incorporated herein in its entirety by reference.

### FIELD OF THE INVENTION

The present invention generally relates to methods and compositions useful for the identification of compounds which modulate the dephosphorylation of a histone deacetylase, and in particular HDAC7, by myosin phosphatase, for inhibiting or inducing apoptosis, and for the treatment of a pathological condition such as smooth muscle cell disorder, cardiac hypertrophy, asthma and other pathological conditions which involve an aberrant expression of a gene under control of an histone deacetylase, in particular HDAC7.

### BACKGROUND OF THE INVENTION

Histone acetylation and deacetylation play essential roles in modifying chromatin structure and regulating gene expression in eukaryotes. Histone deacetylases (HDACs) catalyze the deacetylation of lysine residues in the histone N-terminal tails and are found in large multi-protein complexes with transcriptional co-repressors. Human HDACs are grouped into three classes based on their similarity to known yeast factors. Class I HDACs are similar to the yeast transcriptional repressor yRPD3 and include HDAC1, HDAC2, HDAC3, HDAC8, and HDAC11. They are predominantly nuclear proteins expressed in most tissues and cell lines (Fischle et al., 2001, Biochem Cell Biol 79:337-348). Class II HDACs are related to yHDA1 and include HDAC4, HDAC5, HDAC9, HDAC7, HDAC6N, HDAC10, and HDAC6C. Class III HDACs are similar to ySIR2. Based on sequence homology and domain structure, class II HDACs are further divided into class IIa HDACs (HDAC4, HDAC5, HDAC7, and HDAC9) and class IIb HDACs (HDAC6C, HDAC6N, and HDAC10) (for review, see Verdin et al., 2003, Trends Genet. 19(5):286-293, incorporated herewith by reference in its entirety). These newly discovered enzymes have been implicated as global regulators of gene expression during cell differentiation and development.

Whereas most class I HDACs are ubiquitously expressed, class IIa HDACs are highly similar transcriptional repressors that are expressed in a restricted number of cell types. The repressive activity of Class IIa HDACs is regulated by signal transduction mechanisms that determine whether they are located in the nucleus or cytoplasm (McKinsey et al., 2001, Curr Opin Genet Dev 11:497-504). Three of the class IIa HDACs, HDAC4, -5 and -9, show highest expression in heart, skeletal muscle and brain, where their biological activities might be partially redundant. (Fischle et al., 2001, Biochem Cell Biol 79:337-348; Grozinger et al., 1999, Proc Natl Acad Sci USA 96:4868-4873; Wang et al., 1999, Mol Cell Biol 19:7816-7827; Verdel et al., 1999, J Biol Chem 274:2440-2445; Zhou et al., 2000, Proc Natl Acad Sci USA 97:1056-1061; Zhou et al., 2001, Proc Natl Acad Sci USA 98:1-572-10577). While initial reports described highest HDAC7 expression in heart and lung tissues (Fischle et al., 2001, J Biol Chem 276:35826-35835; Kao et al., 2000, Genes Dev 14:55-66), it was observed that HDAC7 is most highly expressed in CD4/CD8 double-positive thymocytes (Verdin et al., 2003, Trends Genet. 19(5):286-293). In resting thymocytes, HDAC7 is localized in the nucleus and functions as a transcriptional repressor for the proapoptotic orphan receptor Nur77 and other cellular genes involved in T lymphocyte differentiation (Dequiedt et al., 2003, Immunity 18:687-698). After T-cell receptor (TCR) activation or PMA stimulation, the serine/threonine kinase PKD1 phosphorylates HDAC7 on three residues (Serine 155 (S155), Serine 318 (S318), and Serine 448 (S448) that are conserved among other class IIa HDACs (Kao et al., 2001, J Biol Chem 276:47496-47507; Dequiedt et al., 2003, Immunity 18:687-698; Dequiedt et al., 2005, J Exp Med 201:793-804; Parra et al., 2005, J Biol Chem 280:13762-13770). Phosphorylation of HDAC7 leads to its nuclear export, association with 14-3-3 proteins, and to the derepression of its gene targets, including Nur77 (Kao et al., 2001, J Biol Chem 276:47496-47507; Dequiedt et al., 2003, Immunity 18:687-698; Dequiedt et al., 2005, J Exp Med 201:793-804; Parra et al., 2005, J Biol Chem 280:13762-13770).

Histone deacetylases represent the catalytic subunit of large multiprotein complexes. HDACs do not bind directly to DNA and are thought to be recruited to specific promoters through their interaction with DNA sequence-specific transcription factors. Several interacting partners have been described to interact with class II HDACs through distinct domains of class II HDACs. For example, the myocyte enhancer factor 2 (MEF2) family of transcription factors is one of the major targets of class IIa HDACs. For example, HDAC7 in cells is associated with the N-CoR/SMRT complex which contains the histone deacetylase HDAC3 and other associated cofactors. HDAC7 binds indirectly to the transcription factor MEF2, an interaction which targets HDAC7 to selective genes within the human genome. The HDAC7 complex bound at these MEF2 sites deacetylates lysine residues within closely positioned nucleosomes and contributes to transcriptional silencing of the genes occupied by HDAC7. Other HDAC interactions occur with CtBP (E1A C-terminal binding protein), 14-3-3 proteins (a family of highly conserved acidic proteins), calmodulin (CaM), transcriptional co-repressors SMRT (silencing mediator for retinoid and thyroid receptors) and N-CoR (nuclear receptor co-repressor), heterochromatin protein HP1a and SUMO (a ubiquitin-like protein) (for review, see Verdin et al., 2003, Trends Genet. 19(5):286-293; incorporated herewith by reference in its entirety).

Nucleic acid molecules that encode histone deacetylase, in particular HDAC7, as well as recombinant vectors, histone deacetylase polypeptides are disclosed in U.S. Patent Application Nos. 20030143712 and 20060051815, which are incorporated herewith by reference in their entirety.

The many interactions between class IIa HDACs and transcriptional regulators suggest a wide variety of potential biological roles. However, most of these interactions have not been examined in a biological context. By contrast, the importance of interactions between MEF2 and class IIa HDACs has been demonstrated in several tissue culture and animal models. MEF2 plays a significant transcriptional regulatory role in myogenesis, in negative selection of developing thymocytes, and in the transcriptional regulation of Epstein-Barr virus (EBV) (for a complete review see McKinsey et al., 2002 Trends Biochem Sci 27:40-47). Recently, it has been shown that class IIa HDACs inhibit myogenesis by binding to MEF2 at several promoters critical for the muscle differentiation program (McKinsey et al., 2001, Curr Opin Genet Dev 11:497-504; Lu et al., 2000, Proc Natl Acad Sci USA 97:4070-4075).

Central immune tolerance is established in the thymus for T cells via a complex selection process that involves interactions between CD4⁺CD8⁺ double positive thymocytes and antigen-presenting cells. Developing CD4/CD8 double-positive T cells that receive a strong signal from major histocompatibility complex (MHC)-self-peptide through their antigen receptors are deleted by an apoptotic process termed negative selection. The apoptotic process is activated by the expression of Nur77, an orphan steroid receptor (Milbrandt, 1988, Neuron 1:183-188; Hazel et al., Proc Natl Acad Sci USA 85:8444-8448; Ryseck et al., 11989. EMBO J, 8:3327-3335). Constitutive expression of Nur77 in thymocytes results in a dramatic involution of the thymus, whereas expression of a dominant-negative Nur77 interferes with negative selection (Woronicz et al., 1994, Nature 367:277-281; Calnan et al., 1995, Immunity 3, 273-282). HDAC7, a class II histone deacetylase, is highly expressed in CD4⁺ CD8⁺ double positive thymocytes and regulates the expression of genes involved in apoptosis, such as Nur77 (see Verdin et al., 2003, Trends Genet. 19(5):286-293; and herein).

In unstimulated thymocytes, class IIa HDACs, primarily HDAC7, are localized in the nucleus where they associate with a MEF2 family protein, MEF2-D in CD4/CD8 double-positive thymocytes and repress the latent activating potential of MEF2-D, i.e., inhibiting Nur77 expression. After T-cell receptor (TCR) activation elevation of intracellular Ca²⁺ levels activates the Ca²⁺ sensor calmodulin (CaM), which can directly displace class IIa HDACs from MEF2. In addition, CaM-dependent activation of a Ca²⁺/CaM-dependent protein kinase (CaMK) I and II results in the phosphorylation of HDACs. For example, HDAC7, is regulated via the phosphorylation of three serine residues (Ser155, Ser318, and Ser448) by protein kinase D (Parra et al., 2005, J Biol Chem 280(14):13762-70). Ultimately 14-3-3 proteins bind to the phosphorylated class II HDACs and mediate nuclear export of class IIa HDACs. This ultimately allows expression of MEF2 target genes, such as Nur77 and induction of apoptosis (for review, see Verdin et al., 2003, Trends Genet. 19(5):286-293; incorporated herewith by reference in its entirety). Thus, class IIa HDACs, and in particular HDAC7 play a critical role in the repression of Nur77 during thymic maturation of T cells.

Reactivation of latent Epstein Barr Virus (EBV), like myogenesis and Nur77 expression, also seems to be regulated by a Ca²⁺-dependent MEF2 switch in which class IIa HDACs mediate basal repression (Liu et al., 1997, EMBO J. 16:143-153; Gruffat et al., 2002, EMBO Rep 3:141-146).

Upon dephosphorylation by yet unknown cytoplasmic phosphatases, class IIa HDACs, such as HDAC7, are released from 14-3-3 proteins and can reenter the nucleus and shut down MEF2 activated gene expression, such as Nur77 expression and preventing apoptosis (Verdin et al., 2003, Trends Genet. 19(5):286-293).

Myosin phosphatase is a multi-protein complex composed of three subunits: a catalytic subunit of type 1 phosphatase, PP1β, and two regulatory subunits, MYPT1 (myosin phosphatase target subunit) and M20, a smaller subunit of unknown function (for review, see Ito et al., 2004, Mol Cell Biochem 259:197-209; incorporated herein by reference in its entirety). MYPT1 is a critical component of myosin phosphatase targeting the catalytic subunit to a specific substrate. Other MYPT family members have been described and include MYPT2, MBS85, MYPT3 and TIMAP (Ito et al., 2004, Mol Cell Biochem 259:197-209). It has been reported that, for example, MYPT2 is the main myosin phosphatase target subunit expressed in striated muscle (skeletal and cardiac muscle). The activity of myosin phosphatase itself is subject to regulation by phosphorylation and dephosphorylation. For example, phosphorylation of an inhibitory site on MYPT1, Thr696 (human isoform) results in inhibition of PP1c activity. Thr696 in turn can be phosphorylated by, e.g., Rho-kinase. Myosin phosphatase is also inactivated by the protein kinase C-potentiated inhibitor protein 17 kDa (CPI-17). A detailed discussion of (i) the structure of myosin phosphatase (MYPT family members, MYPT isoforms, M20 subunit, catalytic subunits of type 1 phosphatase (PP1c), and subunit interactions), (ii) regulation of myosin phosphatase activity (inhibition of myosin phosphatase by MYPT1 phosphorylation, regulation by subunit dissociation and targeting function, CPI-17, and activation of myosin phosphatase), and (iii) roles of myosin phosphatase in physiological and pathological conditions, see Ito et al. (2004, Mol Cell Biochem 259:197-209; incorporated herewith by reference in its entirety).

The main role assigned to myosin phosphatase has been the dephosphorylation of the phosphorylated myosin light chain (MLC) in smooth muscle cells leading to the relaxation of smooth muscle (Somlyo and Somlyo, 1994, Nature 372:231-236; Somlyo and Somlyo, 1994, J Physiool 552:177-185).

However, to the best of Applicants' knowledge, the role of myosin phosphatase in other cellular systems, as well as the existence of additional substrates has not been described in the prior art. Employing a variety of assays, Applicants herein identify the unknown cytoplasmic phosphatase that dephosphorylates class IIa HDAC, and in particular HDAC7, as myosin phosphatase.

### BRIEF SUMMARY OF THE INVENTION

The present invention relates to screening methods that make use of a histone deacetylase interacting with a myosin phosphatase for the identification of novel therapeutics useful for inhibiting or reducing apoptosis and for inducing apoptosis. Also disclosed are methods for inhibiting or reducing apoptosis and methods for inducing apoptosis in a mammalian cell expressing the histone deacetylase and myosin phosphatase. In addition, methods for the treatment and prevention of smooth muscle cell disorders, cardiac hypertrophy, hypertension, and asthma are disclosed.

In a first aspect, the present invention provides a method for identifying a candidate compound which modulates the dephosphorylation of a histone deacetylase by a myosin phosphatase. In a preferred embodiment, this method comprises the steps of (a) performing a first assay determining the dephosphorylation of a histone deacetylase by a myosin phosphatase and (b) performing a second assay determining the dephosphorylation of the histone deacetylase by the myosin phosphatase in the presence of a candidate compound, wherein the candidate compound which modulates the dephosphorylation of the histone deacetylase is identified. In one embodiment of this invention, this method comprises the step of comparing the result of the first assay to the result of the second assay.

Also provided herein is a method for identifying a candidate compound which modulates the interaction between a histone deacetylase and a myosin phosphatase. In a preferred embodiment of the present invention, this method comprises the steps of (a) performing a first assay determining the interaction between a histone deacetylase and a myosin phosphatase and (b) performing a second assay determining the interaction between the histone deacetylase and the myosin phosphatase in the presence of a candidate compound, wherein the candidate compound which modulates the interaction between the histone deacetylase and the myosin phosphatase is identified. In one embodiment of this invention, this method comprises the step of comparing the result of the first assay to the result of the second assay.

In another aspect of the present invention, a method for identifying a candidate compound capable of reducing or inhibiting apoptosis in a mammalian cell expressing a histone deacetylase, preferably a class II histone deacetylase, is provided. In a preferred embodiment of the present invention, this method comprises the steps of (a) assaying expression of a gene regulated in a mammalian cell by the histone deacetylase and a MEF2 family protein, (b) contacting the mammalian cell with a candidate compound, and (c) determining whether, in the presence of the candidate compound, the expression of the gene regulated by the histone deacetylase and the MEF2 family protein is inhibited, wherein if the expression of the gene in the presence of the candidate compound is inhibited, the candidate compound is a compound for reducing or inhibiting apoptosis.

In yet another aspect of the present invention, a method for identifying a candidate compound for reducing or inhibiting apoptosis is provided. In a preferred embodiment of this method, the method comprises the steps of (a) contacting a myosin phosphatase with a candidate compound, and (b) determining whether the candidate compound binds to the myosin phosphatase, increases the activity of the myosin phosphatase, or increases binding of the myosin phosphatase to a histone deacetylase, wherein the candidate compound that binds to the myosin phosphatase, increases the activity of the myosin phosphatase, or increases binding of the myosin phosphatase to the histone deacetylase is a compound for reducing or inhibiting apoptosis.

The present invention also provides methods for inducing apoptosis. In a preferred embodiment of this invention, this method comprises the steps of (a) contacting a myosin phosphatase with a candidate compound, and (b) determining whether the candidate compound binds to the myosin phosphatase, inhibits the activity of the myosin phosphatase, or inhibits binding of the myosin phosphatase to a histone deacetylase, wherein the candidate compound that binds to the myosin phosphatase, inhibits the activity of the myosin phosphatase, or inhibits binding of the myosin phosphatase to the histone deacetylase is a compound for inducing apoptosis.

In another aspect, the present invention provides a method for identifying a candidate compound which mimics the effect of a myosin phosphatase. In a preferred embodiment of the present invention, this method comprises the steps of (a) assaying an enzymatic activity or binding activity of a histone deacetylase in the presence of a myosin phosphatase, (b) contacting the histone deacetylase with a compound, and (c) determining whether, in the presence of the candidate compound, the histone deacetylase mimics the enzymatic activity or binding activity of the histone deacetylase in the presence of the myosin phosphatase, wherein if the histone deacetylase mimics the enzymatic activity or binding activity of the myosin phosphatase, the candidate compound is a compound that mimics the effect of the myosin phosphatase.

This invention also provides methods for reducing or preventing apoptosis in a mammalian cell expressing a histone deacetylase and a myosin phosphatase. In a preferred embodiment of the present invention, the method comprises the step of contacting the mammalian cell with an effective amount of an agent that increases a level or activity of the myosin phosphatase in the mammalian cell.

The level or activity of the myosin phosphatase in the mammalian cell is increased by at least 10% relative to an untreated control cell, preferably by at least 30% relative to an untreated control cell.

This invention also provides methods for inducing apoptosis in a mammalian cell expressing a histone deacetylase and a myosin phosphatase. In a preferred embodiment of the present invention, the method comprises the step of contacting the mammalian cell with an effective amount of an agent that inhibits the level or activity of the myosin phosphatase in the mammalian cell.

A variety of agents can be used to reduce the level or activity of the myosin phosphatase. A preferred agent is an siRNA. Another preferred agent is an antisense RNA.

According to the present invention, apoptosis can be reduced, inhibited or induced in a mammalian cell. A preferred mammalian cell is a human cell.

Further, apoptosis can be reduced, inhibited or induced in vitro and in vivo. In a preferred embodiment of the present invention, apoptosis is reduced, inhibited, or induced in a human cell which is in a human.

Methods of the present invention can be practiced using a myosin phosphatase from several species. A preferred myosin phosphatase is a human myosin phosphatase.

A variety of histone deacetylases can be used to practice the methods of the invention. A preferred histone deacetylase is a class II histone deacetylase, preferably HDAC7, and more preferably a human HDAC7.

In another aspect of the present invention, a method for the treatment of a pathological condition, which involves an aberrant expression of at least one gene, the expression of which is controlled by a histone deacetylase, preferably a class II histone deacetylase, and a transcription factor of the MEF2 family protein, is provided. In a preferred embodiment of the present invention, this method comprises the step of administering to a patient a therapeutically effective amount of an agent that reduces the interaction between the histone deacetylase and a myosin phosphatase, whereby the expression of at least one gene the expression of which is controlled by a histone deacetylase, preferably a class II histone deacetylase, and a transcription factor of the MEF2 family protein, is increased or decreased, thereby treating the pathological condition.

A preferred gene regulated by HDAC7 is selected from the genes shown in FIG. 5.

A preferred pathological condition is a smooth muscle cell disorder. Other preferred pathological conditions which can be treated using a method of the present invention include cardiac hypertrophy, hypertension, and asthma.

In another aspect, the present invention provides a method for the treatment of cardiac hypertrophy. In a preferred embodiment, this method comprises the steps of (a) identifying a patient having cardiac hypertrophy and (b) administering to the patient an effective amount of an activator of myosin phosphatase, wherein the cardiac hypertrophy is treated.

In another embodiment of the present invention, the method for the treatment of cardiac hypertrophy comprises the steps of (a) identifying a patient having cardiac hypertrophy and (b) administering to the patient an effective amount of an inhibitor of HDAC7 nuclear export, wherein the cardiac hypertrophy is treated.

In a further aspect, this invention also provides a method for the prevention of cardiac hypertrophy. In a preferred embodiment, this method comprises the steps of (a) identifying a patient at risk of developing cardiac hypertrophy and (b) administering to the patient an effective amount of an activator of myosin phosphatase, wherein the cardiac hypertrophy is prevented.

In another embodiment of the present invention, the method for the prevention of cardiac hypertrophy comprises the steps of (a) identifying a patient at risk of developing cardiac hypertrophy and (b) administering to the patient an effective amount of an inhibitor of HDAC7 nuclear export, wherein the cardiac hypertrophy is prevented.

In yet another aspect, the present invention provides a method for the treatment of asthma. In a preferred embodiment, this method comprises the steps of (a) identifying a patient having asthma and (b) administering to the patient an effective amount of an activator of myosin phosphatase, wherein the asthma is treated.

In another embodiment of the present invention, the method for the treatment of asthma comprises the steps of (a) identifying a patient having asthma and (b) administering to the patient an effective amount of an inhibitor of HDAC7 nuclear export, wherein the asthma is treated.

In a further aspect, this invention also provides a method for the prevention of asthma. In a preferred embodiment, this method comprises the steps of (a) identifying a patient at risk of developing asthma and (b) administering to the patient an effective amount of an activator of myosin phosphatase, wherein the asthma is prevented.

In another embodiment of the present invention, the method for the prevention of asthma comprises the steps of (a) identifying a patient at risk of developing asthma and (b) administering to the patient an effective amount of an inhibitor of HDAC7 nuclear export, wherein the asthma is prevented.

Further, this invention provides pharmaceutical compositions comprising compounds and compositions of the present invention. In one aspect, this invention provides pharmaceutical compositions for reducing, inhibiting, or inducing apoptosis. A preferred pharmaceutical composition comprises (i) an agent that modulates the level or activity of a myosin phosphatase and (ii) a pharmaceutically acceptable carrier.

In another aspect, this invention also provides kits comprising compounds and compositions of the present invention. In one aspect, this invention provides kits for reducing, inhibiting, or inducing apoptosis. A preferred kit comprises (i) a container containing an agent that modulates the level or activity of a myosin phosphatase and (ii) instructions for contacting the agent to a mammalian cell for reducing, inhibiting, or inducing apoptosis.

Methods, compositions, and kits of the invention embrace the specifics as described herein.

### BRIEF DESCRIPTION OF THE DRAWINGS

FIG. 1 shows that HDAC7 is predominantly expressed in the thymic cortex. A. Northern blot analysis reveals that HDAC7 was expressed at high level within the thymus. B. In situ hybridization reveals that HDAC7 is expressed in cortical lymphocytes (“C”) within the thymus.

FIG. 2 shows expression of HDAC4, HDAC5, and HDAC7 in various thymocytes populations. HDAC7 is highly expressed in double-positive (CD4⁺ CD8⁺) thymocytes.

FIG. 3 shows that the replacement of the HDAC catalytic domain with the VP 16 activation domain converts HDAC7 from a corepressor to a coactivator. See, Example 3 for details.

FIG. 4 shows that mutation of three serine residues in the N-terminal domain of HDAC7 converts HDAC7 into a super-repressor. Upon TCR activation and phosphorylation of HDAC, HDAC7 disassociates from the MEF2/HDAC7 complex leading to coactivator mediated transcription of target genes. An HDAC7 mutant having the three serine residues mutated and thus, can not be phosphorylated, suppresses target genes. The arrows in the microarray slide indicate genes of which the expression is activated (top) and suppressed (bottom)

FIG. 5 shows genes regulated by HDAC7 in a thymocyte hybridoma. For details, see Example 3.

FIG. 6 shows that phosphorylation of HDAC7 by PMA is transient. A. HDAC7 was immunoprecipitated using an α-HDAC7 antibody and analyzed by Western blotting using anti-phospho serine specific HDAC7 antibodies (α-P-Ser155; α-P-Ser318, α-P-Ser448; α-FLAG was used as a control). An anti-Flag Western blot shows equal amounts of HDAC7-Flag were immunoprecipitated. CIP, treatment of the immunoprecipitated material with phosphatase before Western blotting. B. DO11.10-HDAC-Flag cells were treated with PMA alone (left panel) or with PMA+okadaic acid (right panel) for the times indicated. Equal amounts of HDAC7-Flag was immunoblotted with antiphospho-HDAC7 antisera. C. DO11.10-HDAC7-Flag cells were treated or not with anti CD3 antibody for the indicated times. HDAC7 phosphorylation was analyzed with antiphospho-HDAC7 antisera as in B. For details, see Example 4.

FIG. 7 shows that the nuclear exclusion of HDAC7 following PMA treatment is transient. A. Immunofluorescence was performed in DO11.10 cells nucleofected with an HDAC7-GFP expression vector followed 24 h later by PMA treatment for the indicated times. HDAC7 subcellular distribution was analyzed by immunofluorescence microscopy (representative fields are shown. B. Quantitation of immunofluorescence microscopy in A. The percentage of cells showing nuclear exclusion of HDAC7 is indicated at each time point. One-hundred cells were counted for each point. Error bars represent SEM for four independent experiments. For details, see Example 5.

FIG. 8 shows that activation of Nur77 by PMA is transient. A. Western blot showing total cell lysates prepared from DO11.10 cells, treated with PMA as in FIG. 6B were analyzed by Western blotting with antisera against Nur77 and actin. B. Western blot showing total cell lysates prepared from DO11.10 cells, treated with CD3 antibodies as in FIG. 6C, were analyzed by Western blotting with antisera against Nur77 and actin. For details, see Example 6.

FIG. 9A depicts a Coomassie gel of HDAC7-Flag-tagged-containing complexes immunoprecipitated from DO11.101 cells with anti-M2 agarose beads leading to the identification of an HDAC7 associated phosphatase. Lane 1, size marker; lane 2 (Empty), T cells transfected with empty vector; lane 3 (HDAC7-FLAG), T cells transfected with vector encoding FLAG-HDAC7. The positions of HDAC7 interacting proteins MYPT1, HDAC7, PP1β, 14-3-3β, 14-3-3ε, and 14-3-3θ, identified by mass spectrometry, are indicated by arrows. FIG. 9B depicts Western blotting analysis showing HDAC7 was immunoprecipitated from DO11.10-HDAC7-FLAG T cells and probed for its association with various proteins using the antibodies indicated. Details are described in Example 7.

FIG. 10 depicts interaction of myosin phosphatase with HDAC7 in mouse primary thymocytes as shown by immunoprecipitation and Western blot analysis. A. Proteins were immunoprecipitated from total cell lysates prepared from mouse primary thymocytes using α-PP1β antibodies or no antibody (control) and probed for its association with HDAC7 using an anti-HDAC7 antibody. B. Proteins were immunoprecipitated from total cell lysates prepared from mouse primary thymocytes using α-PP1β antibodies, α-PP1γ antibodies, α-MYTP1 antibodies or α-14-3-3ε antibodies or no antibody (control) and probed for its association with HDAC7 by immunoblotting with a anti-HDAC7 antibody. Details are described in Example 8.

FIG. 11 shows that myosin phosphatase (subunit PP1β) dephosphorylates HDCA7. A. DO11.10-HDAC7-Flag cells were either left untreated or treated with PMA for 30 min. A mixture of recombinant PP1 isoforms was added to the immunoprecipitated material before Western blotting. After stimulation by PMA, HDAC7 serine residues at position 155, 318, and 448 become phosphorylated as shown by Western blot analysis using specific anti-HDAC7 phospho antibodies α-P-Ser155, α-P-Ser318, and α-P-Ser448. α-Flag was used as a control. B. DO11.10-HDAC7-Flag cells nucleofected with either siCo or siPP1β+MYPT1 were treated with PMA for the indicated times. HDAC7 phosphorylation was determined as described in FIG. 6. HDAC7-Flag, PP1β and MYPT1 protein levels are shown. Details are described in Example 9.

FIG. 12 shows that suppression of myosin phosphatase by RNAi in DO11.10 cells enhances HDAC7 exclusion from the nucleus and delays HDAC7 re-entry into the nucleus. A. SiRNA treatment reduces cellular PP1β and MYPT1 proteins. B. SiRNA treatment leads to exportation of HDAC7 into the cytoplasm followed by relocalization to the nucleus. When PP1β and MYPT1 are both knocked down, this re-entry is significantly delayed. The graph represents the percentage of cells where HDAC7 was excluded from the nucleus. siCo, treatment of cells with control siRNAs; siRNAPP1β+MYTP1, treatment of cells with siRNAs specific for PP1β and MYTP1. “*” means statistically significant. Error bars represent SEM for four independent experiments. For details, see Example 10.

FIG. 13 shows that suppression of myosin phosphatase by RNAi in DO11.10 cells induces Nur77 expression and apoptosis in mouse thymocytes. A. Depletion of MYPT1, PP1β, or both in DO11.10 cells by siRNA-mediated knockdown. PP1β and MYPT1 protein levels were analyzed 48 h after nucleofection of the different siRNAs. B. Myosin phosphatase regulates Nur77 induction. DO11.10-Empty cells (expressing HDAC7) or DO11.10-HDAC7ΔP cells (expressing an HDAC7 phosphorylation mutant) were nucleofected with either siCo, siPP1β, siMYPT1, or siPP1β+siMYPT1. After 24 h, cells were induced with α-CD3 antibodies in the absence (−) or presence (+) of siRNAs as indicated. Cellular extracts were prepared and expression of Nur77 was analyzed by Western blotting using an anti-Nur77 antibody (α-Nur77). A-Actin was used as a control. C. Depletion of PP1α, PP1β, and PP1γ in DO11.10 cells by siRNA-mediated knockdown. PP1α, PP1β, and PP1γ protein levels were analyzed 48 h after nucleofection with different siRNAs. D. DO11.10-Empty or DO11.10-HDAC7ΔP-Flag cells were nucleofected with siRNAs for the different PP1 isoforms. Cells were treated and analyzed as above. Details are described in Example 11.

FIG. 14 demonstrates that myosin phosphatase regulates apoptosis in mouse thymocytes. The indicated siRNAs were introduced into primary thymocytes. Cells were treated with anti-CD3 antibody for 16 h, followed by staining with anti-CD4-PE, anti-CD8-FTIC and AnnexinV-APC followed by flow cytometry analysis. A representative FACS plot is shown that quantifies Annexin V binding (y axis) in CD4⁺CD8⁺ cells, i.e apoptosis. Data are represented as mean±SEM of three independent experiments. “*” p<0.001. In the upper panel, flow histograms illustrate the percentage of apoptotic cells in a representative experiment. Details are described in Example 12.

FIG. 15 shows a role of HDAC7 in thymocyte differentiation. A. Under basal conditions no effect of any of the constructs (HDAC7-VP 16 or HDAC7Δ) was observed and the cells were maintained as double positive CD4 and CD8. B. When the cells were cocultivated with the antigen presenting cells DCEK-ICAM, HDAC7-VP 16 fusion protein expression was associated with a very significant differentiation of the cells into single positive CD4 T cells. C. When the peptide was added, the cells also became differentiated in CD4 positive T cells, but this effect was largely suppressed by the expression of the HDAC7 superrepressor HDAC7-ΔP. The fax plots show CD8 is on the X axis while CD4 is on the Y axis. Details are described in Example 13.

FIG. 16 shows a model for thymocyte differentiation and the roles of HDAC7 and myosin phosphatase. A. Signaling pathways responsible for HDAC7 nucleocytoplasmic shuttling after TCR activation. The nucleocytoplasmic location of HDAC7 is under the competing influences of a kinase (PKD1) and a phosphatase (myosin phosphatase). B. TCR activation leads to the functional inactivation of HDAC7 (indicated by crossing out) and gene activation. Details are described in Example 14.

### DETAILED DESCRIPTION OF THE INVENTION

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, “activity of myosin phosphatase” refers to (i) the binding of myosin phosphatase to a polypeptide or peptide, (ii) the interaction of a myosin phosphatase with a polypeptide or peptide, or (iii) the dephosphorylation of a phosphorylated polypeptide or phosphorylated peptide.

As used herein, “antagonist” means a chemical substance that diminishes, abolishes or interferes with the physiological action of a polypeptide. The antagonist may be, for example, a chemical antagonist, a pharmacokinetic antagonist, a non-competitive antagonist, or a physiological antagonist, such as a biomolecule, e.g., a polypeptide. A preferred antagonist diminishes, abolishes or interferes with the physiological action or activity of a myosin phosphatase.

Specifically, an antagonist may act at the level of the interaction between a first polypeptide, e.g., a myosin phosphatase and a second polypeptide, for example, a binding partner, such as a histone deacetylase. The antagonist, for example, may competitively or non-competitively (e.g., allosterically) inhibit binding of the first polypeptide to the second polypeptide. A “competitive antagonist” is a molecule which binds directly to the first polypeptide in a manner that sterically interferes with the interaction of the first polypeptide with the second polypeptide. Non-competitive antagonism describes a situation where the antagonist does not compete directly with the binding, but instead blocks a point in the signal transduction pathway subsequent to the binding of the first polypeptide to the second polypeptide. A “pharmacokinetic antagonist” effectively reduces the concentration of the active drug at its site of action, e.g., by increasing the rate of metabolic degradation of the first polypeptide. Physiological antagonism loosely describes the interaction of two substances whose opposing actions in the body tend to cancel each other out. An antagonist can also be a substance that diminishes or abolishes expression of a first polypeptide. Thus, a myosin phosphatase antagonist can be, for example, a substance that diminishes or abolishes: (i) the expression of the gene encoding myosin phosphatase, (ii) the translation of myosin phosphatase RNA, (iii) the post-translational modification of myosin phosphatase, or (iv) the interaction of subunits of the myosin phosphatase to form a functional myosin phosphatase.

The term “antisense-oligonucleotides” as used herein encompasses both nucleotides that are entirely complementary to a target sequence and those having a mismatch of one or more nucleotides, so long as the antisense-oligonucleotides can specifically hybridize to the target sequence. For example, the antisense-oligonucleotides of the present invention include polynucleotides that have a homology (also referred to as sequence identity) of at least 70% or higher, preferably at 80% or higher, more preferably 90% or higher, even more preferably 95% or higher over a span of at least 15 continuous nucleotides up to the full length sequence of any of the nucleotide sequences of a PP1α, PP1β, PP1γ, MYPT1 or M20 gene. Algorithms known in the art can be used to determine the homology. Furthermore, derivatives or modified products of the antisense-oligonucleotides can also be used as antisense-oligonucleotides in the present invention. Examples of such modified products include lower alkyl phosphonate modifications such as methyl-phosphonate-type or ethyl-phosphonate-type, phosphorothioate modifications and phosphoroamidate modifications.

As used herein, “biological sample” means a sample of biological tissue or fluid that contains nucleic acids and/or polypeptides. Such samples are typically from humans, but include tissues isolated from non-human primates, or rodents, e.g., mice, and rats. Biological samples may also include sections of tissues such as biopsy and autopsy samples, frozen sections taken for histological purposes, cerebral spinal fluid, blood, plasma, serum, sputum, stool, tears, mucus, hair, skin, etc. Biological samples also include explants and primary and/or transformed cell cultures derived from patient tissues. A “biological sample” also refers to a cell or population of cells or a quantity of tissue or fluid from an animal. Most often, the biological sample has been removed from an animal, but the term “biological sample” can also refer to cells or tissue analyzed in vivo, i.e., without removal from the animal. Typically, a “biological sample” will contain cells from the animal, but the term can also refer to noncellular biological material, such as noncellular fractions of blood, serum, saliva, cerebral spinal fluid or urine, that can be used to measure expression level of a polynucleotide or polypeptide. Numerous types of biological samples can be used in the present invention, including, but not limited to, a tissue biopsy or a blood sample. As used herein, a “tissue biopsy” refers to an amount of tissue removed from an animal, preferably a human, for diagnostic analysis. “Tissue biopsy” can refer to any type of biopsy, such as needle biopsy, fine needle biopsy, surgical biopsy, etc.

As used herein, “providing a biological sample” means to obtain a biological sample for use in methods described in this invention. Most often, this will be done by removing a sample of cells from a subject, but can also be accomplished by using previously isolated cells (e.g., isolated by another person, at another time, and/or for another purpose), or by performing the methods of the invention in vivo. Archival tissues, having treatment or outcome history, will be particularly useful.

The terms “candidate agent,” “agent”, “candidate compound” “compound” and “small molecule” are used interchangeably herein. Candidate agents encompass numerous chemical classes, typically synthetic, semi-synthetic, or naturally-occurring inorganic or organic molecules. Candidate agents may be small organic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

As used herein, the term “cardiac hypertrophy” refers to the process in which adult cardiac myocytes respond to stress through hypertrophic growth. Such growth is characterized by cell size increases without cell division, assembling of additional sarcomeres within the cell to maximize force generation, and activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the term “decreased expression” refers to a level of a gene expression product that is lower and/or the activity of the gene expression product is lower. Preferably, the decrease is at least 20%, more preferably, the decrease is at least 30%, at least 40%, at least 50%, at least 60%, at least 70%, at least 80%, or at least 90% and most preferably, the decrease is at least 100%, relative to a control.

Synonyms of the term, “determining” are contemplated within the scope of the present invention and include, but are not limited to, detecting, measuring, assaying, or testing for the presence, absence, amount or concentration of a molecule, such as a myosin phosphatase, a histone deacetylase, a label, a small molecule of the invention or a myosin phosphatase antagonist. The term refers to both qualitative and quantitative determinations.

As used herein, “determining the functional effect” means assaying for a compound that increases or decreases a parameter that is indirectly or directly under the influence of the compound, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of a gene, such as Nur77, measuring binding activity, e.g., binding of a myosin phosphatase to a histone deacetylase, assaying for phosphorylation and/or dephosphorylation of e.g., a histone deacetylase, measuring cellular proliferation, measuring apoptosis, measuring subcellular localization of a polypeptide, such as histone deacetylase, or the like. Determination of the functional effect of a compound on a disease, disorder, cancer or other pathology can also be performed using assays known to those of skill in the art such as in vitro assays, e.g., cellular proliferation; growth factor or serum dependence; mRNA and protein expression in cells, and other characteristics of cells. The functional effects can be evaluated by many means known to those skilled in the art, e.g., microscopy for quantitative or qualitative measures of alterations in morphological features, measurement of changes in RNA or protein levels, measurement of RNA stability, identification of downstream or reporter gene expression (CAT, luciferase, β-gal, GFP and the like), e.g., via chemiluminescence, fluorescence, colorimetric reactions, antibody binding, inducible markers, ligand binding assays, apoptosis assays, and the like. “Functional effects” include in vitro, in vivo, and ex vivo activities.

As used herein, “disorder”, “disease” or “pathological condition” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

As used herein, “effective amount”, “effective dose”, “sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition. In some embodiments, the desired result is an increase in nuclear localization of a histone deacetylase. In other embodiments, the desired result is an increase in cytoplasmic localization of a histone deacetylase. In yet other embodiments, the desired result is an increase or decrease in the phosphorylation status of a histone deacetylase. Amelioration of a symptom of a particular condition by administration of a pharmaceutical composition described herein refers to any lessening, whether permanent or temporary, lasting or transient that can be associated with the administration of the pharmaceutical composition. An “effective amount” can be administered in vivo and/or in vitro.

As used herein, “HDAC” means histone deacetylase.

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

For the purposes of this invention the terms “hybridize” or “hybridize specifically” are used to refer to the ability of two nucleic acid molecules to hybridize under “stringent hybridization conditions.” The phrase “stringent hybridization conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times, background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 50° C. The antisense-oligonucleotides and derivatives thereof act on cells producing the proteins encoded by a PP1α, PP1β, PP1γ, MYPT1, or M20 gene by binding to the DNA or mRNA encoding the protein, inhibiting transcription or translation thereof, promoting the degradation of the mRNAs and inhibiting the expression of the protein, thereby resulting in the inhibition of the protein function.

The terms “individual,” “subject,” “host,” and “patient,” used interchangeably herein, refer to a mammal, including, but not limited to, murines, simians, felines, canines, equines, bovines, mammalian farm animals, mammalian sport animals, and mammalian pets and humans.

As used herein, “in vitro” means outside the body of the organism from which a cell or cells is obtained or from which a cell line is isolated.

As used herein, “in vivo” means within the body of the organism from which a cell or cells is obtained or from which a cell line is isolated.

A “label” or a “detectable moiety” is a composition detectable by spectroscopic, photochemical, biochemical, immunochemical, chemical, or other physical means. For example, useful labels include ³H, ¹²⁵I, ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a histone deacetylase or a small molecule compound. A preferred label is ³²P.

As used herein, “level of an mRNA” in a biological sample refers to the amount of mRNA transcribed from a gene that is present in a cell or a biological sample. The mRNA generally encodes a functional protein, although mutations may be present that alter or eliminate the function of the encoded protein. A “level of mRNA” need not be quantified, but can simply be detected, e.g., a subjective, visual detection by a human, with or without comparison to a level from a control sample or a level expected of a control sample. A preferred mRNA is a myosin phosphatase mRNA, a histone acetylase mRNA or a Nur77 mRNA.

As used herein, “level of a polypeptide” in a biological sample refers to the amount of polypeptide translated from an mRNA that is present in a cell or biological sample. The polypeptide may or may not have protein activity. A “level of a polypeptide” need not be quantified, but can simply be detected, e.g., a subjective, visual detection by a human, with or without comparison to a level from a control sample or a level expected of a control sample. A preferred polypeptide is a myosin phosphatase polypeptide, a histone acetylase polypeptide or a Nur77 polypeptide.

As used herein, “mammal” or “mammalian” means or relates to the class mammalia including the orders carnivore (e.g., dogs and cats), rodentia (e.g., mice, guinea pigs, and rats), and primates (e.g., humans, chimpanzees, and monkeys).

As used herein, the term “modulate” encompasses “increase” and “decrease.” In some embodiments, of particular interest are agents which inhibit myosin phosphatase activity, and/or which reduce a level of a myosin phosphatase polypeptide in a cell, and/or which reduce a level of a myosin phosphatase mRNA in a cell. In other embodiments, of particular interest are agents which increase myosin phosphatase activity, and/or which increase a level of a myosin phosphatase polypeptide in a cell, and/or which increase a level of a myosin phosphatase mRNA in a cell. Such agents are of interest as candidates for reducing, inhibiting or inducing apoptosis and for treating a pathological condition, e.g., cancer, cardiac hypertrophy, hypertension, or asthma.

As used herein a “modulator” of the level or activity of a polypeptide, such as a myosin phosphatase, includes an activator and/or inhibitor of that polypeptide and is used to refer to agents that activate or inhibit the level of expression of the polypeptide or the activity of the polypeptide. A preferred polypeptide is myosin phosphatase. Another preferred polypeptide is a histone deacetylase. Activators are agents that, e.g., induce or activate the expression of a polypeptide of the invention or bind to, stimulate, increase, open, activate, facilitate, or enhance activation, sensitize or up regulate the activity of a polypeptide of the invention. Activators include nucleic acids that encode myosin phosphatase, demethylating compounds, as well as naturally occurring and synthetic compounds, small chemical molecules and the like. Assays for activators include, e.g., applying candidate compounds to cells expressing myosin phosphatase and histone deacetylase and then determining the functional effects. Samples or assays comprising myosin phosphatase and histone deacetylase that are treated with a potential activator are compared to control samples without the activator to examine the extent of effect. Control samples (untreated with candidate agents) are assigned a relative activity value of 100%. Activation of the polypeptide is achieved when the polypeptide activity value relative to the control is 110%, optionally 130%, 150%, optionally 200%, 300%, 400%, 500%, or 1000-3000% or more higher. Inhibitors are agents that, e.g., repress or inactivate the expression of a polypeptide of the invention or bind to, decrease, close, inactivate, impede, or reduce activation, desensitize or down regulate the activity of a polypeptide of the invention. Inhibitors include nucleic acids such as siRNA and antisense RNA that interfere with the expression of myosin phosphatase, as well as naturally occurring and synthetic compounds, small chemical molecules and the like. Assays for activators (see above and herein) can also be used as assays for inhibitors. Samples or assays comprising myosin phosphatase and histone deacetylase that are treated with a potential inhibitor are compared to control samples without the inhibitor to examine the extent of effect. Control samples (untreated with candidate agents) are assigned a relative activity value of 100%. Inhibition of the polypeptide is achieved when the polypeptide activity value relative to the control is reduced by 10%, optionally 20%, optionally 30%, optionally 40%, optionally 50%, 60%, 70%, 80%, or 90-100%.

As used herein, “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, “polypeptide,” and “protein” are used interchangeably herein to refer to a polymer of amino acid residues. The terms also apply to amino acid polymers in which one or more amino acid residue is an artificial chemical mimetic of a corresponding naturally occurring amino acids, as well as to naturally occurring amino acid polymers, those containing modified residues, and non-naturally occurring amino acid polymers.

The term “recombinant” when used with reference to, e.g., a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

As used herein, the term “salts” refers to salts of an active compound of the present invention, such as a myosin phosphatase antagonist, which are prepared with relatively nontoxic acids or bases, depending on the particular substituents found on the compounds described herein. When compounds, agents, and small molecules of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds, agents, and small molecules of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds, agents, and small molecules of the present invention may be regenerated by contacting the salt with a base or acid and isolating the parent compound in the conventional manner. The parent form of the compound, agent, and small molecule differs from the various salt forms in certain physical properties, such as solubility in polar solvents, but otherwise the salts are equivalent to the parent form of the compound, agent, and small molecule for the purposes of the present invention.

By “small interfering RNA,” “short interfering RNA,” or “siRNA” is meant an isolated RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that functions as a key intermediate in triggering sequence-specific RNA degradation. A range of 19-25 nucleotides is the most preferred size for siRNAs. siRNAs can also include short hairpin RNAs (shRNA) in which both strands of an siRNA duplex are included within a single RNA molecule. Double-stranded siRNAs generally consist of a sense and anti-sense strand. Single-stranded siRNAs generally consist of only the antisense strand that is complementary to the target gene or mRNA. siRNA includes any form of RNA, preferably dsRNA (proteolytically cleaved products of larger dsRNA, partially purified RNA, essentially pure RNA, synthetic RNA, recombinantly produced RNA) as well as modified RNA that differs from naturally occurring RNA by the addition, deletion, substitution, and/or alteration of one or more nucleotides.

As used herein, the term “solvate” refers to compounds, agents, and small molecules of the present invention that are complexed to a solvent. Solvents that can form solvates with the compounds, agents, and small molecules of the present invention include common organic solvents such as alcohols (methanol, ethanol, etc.), ethers, acetone, ethyl acetate, halogenated solvents (methylene chloride, chloroform, etc.), hexane and pentane. Additional solvents include water. When water is the complexing solvent, the complex is termed a “hydrate.”

As used herein, “subject” or “patient” to be treated for a pathological condition or disease by a method of the present invention means either a human or non-human animal in need of treatment for a pathological condition or disease.

As used herein, the terms “treat”, “treating”, and “treatment” include: (1) preventing a pathological condition or disease, i.e. causing the clinical symptoms of the pathological condition or disease not to develop in a subject that may be predisposed to the pathological condition or disease but does not yet experience any symptoms of the pathological condition or disease; (2) inhibiting the pathological condition or disease, i.e. arresting or reducing the development of the pathological condition or disease or its clinical symptoms; or (3) relieving the pathological condition or disease, i.e. causing regression of the pathological condition or disease or its clinical symptoms. These terms encompass also prophylaxis, therapy and cure. Treatment means any manner in which the symptoms of a pathological condition or disease are ameliorated or otherwise beneficially altered. Preferably, the subject in need of such treatment is a mammal, more preferable a human.

### II. Inhibitors of Myosin Phosphatase

The present invention relates to screening methods that make use of a histone deacetylase interacting with a myosin phosphatase for the identification of novel therapeutics useful for inhibiting or reducing apoptosis and for inducing apoptosis. Also disclosed are methods for inhibiting or reducing apoptosis and methods for inducing apoptosis in a mammalian cell expressing the histone deacetylase and myosin phosphatase. Applicants have discovered that myosin phosphatase binds to and dephosphorylates a histone deacetylase, such as a class II histone deacetylase, and in particular HDAC7. As described herein, dephosphorylation of a histone deacetylase, such as HDAC7, leads to the inhibition of apoptosis. Thus, compounds that modulate the level or activity of a myosin phosphatase, and in particular the dephosphorylation of a histone deacetylase by a myosin phosphatase or the interaction between a histone deacetylase and a myosin phosphatase, are useful for inhibiting, reducing or inducing apoptosis. Compounds that inhibit the level or activity of a myosin phosphatase are particularly useful for inducing apoptosis.

A. SiRNA

A variety of compounds can be used to inhibit the level or activity of a myosin phosphatase and in particular inhibit the dephosphorylation of a histone deacetylase by the myosin phosphatase or inhibit the interaction between a histone deacetylase and the myosin phosphatase. In a preferred embodiment the inhibitor is a small interfering RNA (siRNA). See, e.g., PCT applications WO00/44895, WO99/32619, WO01/75164, WO01/92513, WO01/29058, WO01/89304, WO02/16620, and WO02/29858; and U.S. Patent Publication No. 20040023390 for descriptions of siRNA technology. In a preferred embodiment the inhibitor is an siRNA directed against a myosin phosphatase mRNA, more specifically against a PP1β mRNA, against a MYPT1 mRNA or against a M20 mRNA.

Agents of the present invention that are useful for practicing the methods of the present invention include, but are not limited to siRNAs of myosin phosphatase. Typically, such agents are capable of (i) binding to myosin phosphatase mRNA, (ii) interfere with translation of myosin phosphatase mRNA or (iii) lead to degradation of myosin phosphatase mRNA. In a preferred embodiment, the agent inhibiting the level or activity of myosin phosphatase is an siRNA of myosin phosphatase. The present invention provides compositions and methods using RNA interference to modulate myosin phosphatase expression. These methods and compositions are useful for the treatment of pathological conditions, disease, induction of apoptosis and interfering with myosin phosphatase activity.

In many species, introduction of double-stranded RNA (dsRNA) which may alternatively be referred to herein as small interfering RNA (siRNA), induces potent and specific gene silencing, a phenomena called RNA interference or RNAi. This phenomenon has been extensively documented in the nematode C. elegans (Fire et al., 1998, Nature, 391:806-811), but is widespread in other organisms, ranging from trypanasomes to mouse. Depending on the organism being discussed, RNA interference has been referred to as “cosuppression”, “post-transcriptional gene silencing”, “sense suppression” and “quelling.” RNAi is an attractive biotechnological tool because it provides a means for knocking out the activity of specific genes. It is particularly useful for knocking out gene expression in species that were not previously considered to be amenable to genetic analysis or manipulation.

RNAi is usually described as a post-transcriptional gene-silencing (PTGS) phenomenon in which dsRNAs trigger degradation of homologous mRNA in the cytoplasm. The basic process involves a dsRNA that is processed into shorter units (called short interfering RNAs (siRNAs)) that guide recognition and targeted cleavage of homologous messenger RNA (mRNA). The dsRNAs that (after processing) trigger RNAi/PTGS can be made in the nucleus or cytoplasm in a number of ways. The processing of dsRNA into siRNAs, which in turn degrade mRNA, is a two-step RNA degradation process. The first step involves a dsRNA endonuclease (ribonuclease III-like; RNase III-like) activity that processes dsRNA into sense and antisense RNAs which are 21 to 25 nucleotides (nt) long (i.e., siRNA). In Drosophila, this RNase III-type protein is termed Dicer. In the second step, the antisense siRNAs produced combine with, and serve as guides for, a different ribonuclease complex called RNA-induced silencing complex (RISC), which cleaves the homologous single-stranded mRNAs. RISC cuts the mRNA approximately in the middle of the region paired with the antisense siRNA, after which the mRNA is further degraded. dsRNAs from different sources can enter the processing pathway leading to RNAi/PTGS.

Thus, in a preferred embodiment of the present invention, the agent for use in the methods of the present invention is an siRNA of myosin phosphatase. siRNA can be used to reduce the expression level of myosin phosphatase. An siRNA of myosin phosphatase hybridizes to a myosin phosphatase mRNA and thereby decreases or inhibits production of myosin phosphatase protein.

In designing RNAi experiments there are several factors that need to be considered such as the nature of the siRNA, the durability of the silencing effect, and the choice of delivery system. To produce an RNAi effect, the siRNA that is introduced into the organism should contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 90% or more prefererably 100% identity between the sequence of the siRNA and the sequence of the gene to be inhibited. Sequences with less than about 80% identity to the target gene are substantially less effective. Thus, the greater homology between the siRNA of myosin phosphatase and the myosin phosphatase gene whose expression is to be inhibited, the less likely expression of unrelated genes will be affected.

In addition, the size of the siRNA is important. Generally, the present invention relates to siRNA molecules of myosin phosphatase, which are double or single stranded and comprise at least about 19-25 nucleotides, and are able to modulate the gene expression of myosin phosphatase. In the context of the present invention, the siRNA is preferably less than 500, 200, 100, 50 or 25 nucleotides in length. More preferably, the siRNA is from about 19 nucleotides to about 25 nucleotides in length.

In one aspect, the invention generally features an isolated siRNA molecule of at least 19 nucleotides, having at least one strand that is substantially complementary to at least ten but no more than thirty consecutive nucleotides of myosin phosphatase, and that reduces the expression of myosin phosphatase gene or protein.

Applicants have described useful siRNA sequences herein for the inhibition of myosin phosphatase (siMYPT1) and PP1β (siPP1β). One of ordinary skill in the art would appreciate that agents inhibiting MYPT1 or PP1β expression are not limited to the siRNA sequences disclosed herein. Rather, one of skill in the art would appreciate that nucleic acids inhibiting MYPT1 or PP1β expression include, e.g., MYPT1 or PP1β small interfering RNA (siRNA), MYPT1 or PP1β micro RNA (miRNA), MYPT1 or PP1β short hairpin RNA (shRNA) and MYPT1 or PP1β antisense RNA, and the like.

Numerous publications have provided guidance for the design of siRNA, miRNAs, shRNAs or antisense RNAs. For example, Elbashir et al., (Elbashir et al., 2001, EMBO J, 20(23):6877-6888) teaches synthetic, short interfering RNAs (siRNAs) and their requirement regarding length, structure, chemical composition and sequence in order to mediate efficient RNA interference. Elbashir et al. (Elbashir et al. 2002, Methods 26(2): 199-213), provides a collection of protocols for siRNA-mediated knockdown of mammalian gene expression and eludes to the “robustness of the siRNA knockdown technology.” Additional guidance for the design of siRNAs is provided by Amarzguioui et al. (Amarzguioui et al., 2003, Nucl Acids Res (31(2):589-595). Further, Harborth et al. (Harborth et al., 2003, Antisense Nucleic Acid Drug Dev. 13(2):83-105) address the predictability of siRNA inhibition and find that 26 of 44 tested standard 21-23 nucleotide (nt) siRNA duplexes reduced protein expression by at least 90%, and only two duplexes reduced protein expression to <50%. Also Semizarov et al. (Semizarov et al. 2003, Proc Natl Acad Sci USA, 100(11):6347-6352) conclude that siRNA is a highly specific tool for targeted gene knockdown. Thus, the state of the art of designing nucleic acids, such as siRNAs, based on a known target sequence, for efficient inhibition of a target protein expression and the level of ordinary skill is high. Further, there is a high predictability in the art.

Myosin phosphatase is a complex of three components as described herein, PP1β, MYPT1, and M20. Thus, referring to an siRNA inhibiting expression of a myosin phosphatase means an siRNA for PP1β, MYPT1 and/or M20 leading to the inhibition of expression of PP1, MYPT1 and/or M20. Nucleic acid sequences encoding those subunits are described in the art and are available at Genbank. For example, human nucleic acid and protein sequences for MYPT1 can be found, e.g., at GenBank Accession Nos. D87930 and AF458589; mouse MYPT1 sequences can be found, e.g., at Genbank Accession No. AB042280. Having these sequences at hand, a skilled artisan can readily identify without undue experimentation by using, e.g., the disclosure provided herein, siRNAs other than those disclosed herein, for practicing methods and compositions of the present invention.

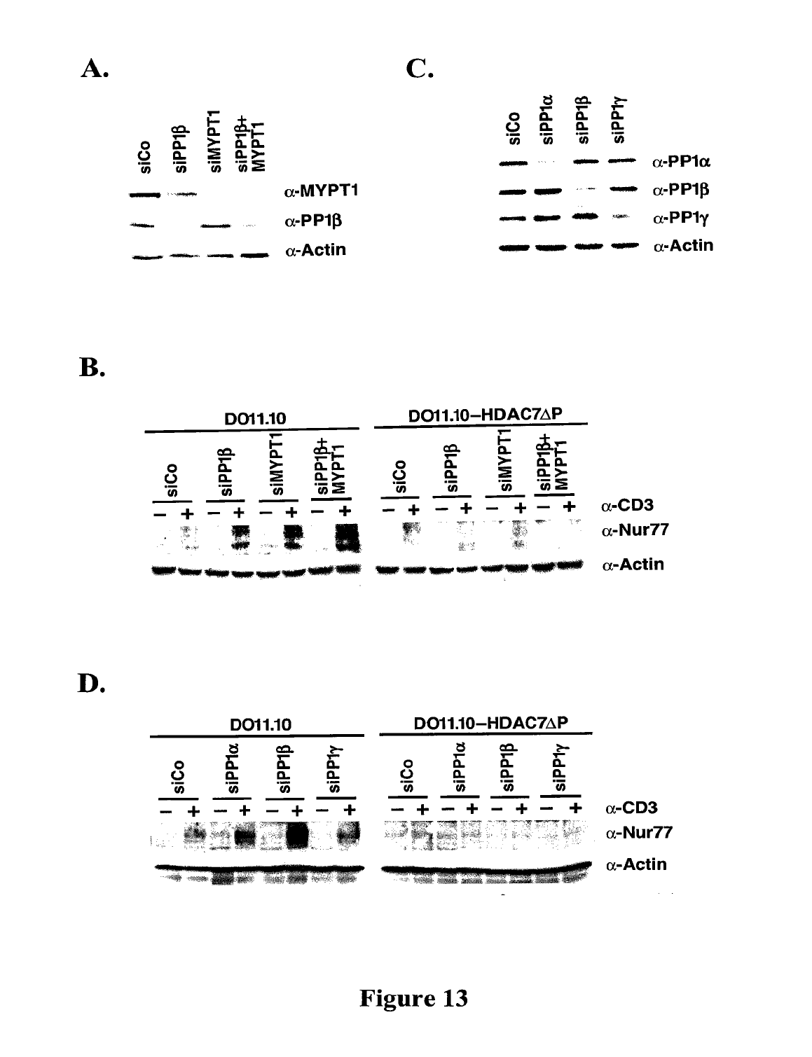

In a preferred embodiment of the present invention, the siRNA molecule has at least one strand that is substantially complementary to at least ten but no more than thirty consecutive nucleotides of a PP1β. In a preferred embodiment, the siRNA for inhibiting PP1β expression from PP1β mRNA (siPP1β) comprises the following nucleic acid sequence: 5′-CCAGAAGCCAACUAUCUUU-3′. In another preferred embodiment, the siRNA for inhibiting PP1β expression from PP1β mRNA (siPP1β) comprises the following nucleic acid sequence: 5′-GCCAACUAUCUUUUCUUAG-3′. In yet another preferred embodiment, the siRNA for inhibiting PP1β expression from PP1β mRNA (siPP1β) comprises the following nucleic acid sequence: 5′-CGGAUAUGAAUUUUUUGCU-3′. Other useful siRNAs for inhibiting PP1β are 5′-CCAGAAGCCAACUAUCUUUtt-3′,5′-GCCAACUAUCUUUUCUUAGtt-3′ and 5′-CGGAUAUGAAUUUUUUGCUtt-3′. The siPP1β oligonucleotides may be used alone or in combination with each other to inhibit PP1β expression. Using these oligonucleotides, expression of PP1β has been drastically reduced (Examples 10 and 11; FIGS. 12 and 13).

In another embodiment of the present invention, an siRNA is used for inhibiting expression of the MYPT1 subunit of the myosin phosphatase. In a preferred embodiment of the present invention, the siRNA molecule has at least one strand that is substantially complementary to at least ten but no more than thirty consecutive nucleotides of a MYPT1. In a preferred embodiment, the siRNA for inhibiting MYPT1 expression from MYPT1 mRNA (siMYPT) comprises the following nucleic acid sequence: 5′-GAACGAGACUUGCGUAUGUUU-3′. In another preferred embodiment, the siRNA for inhibiting MYPT1 expression from MYPT1 mRNA (siMYPT1) comprises the following nucleic acid sequence: 5′-AAGAAUAGUUCGAUCAAUGUU-3′. In another preferred embodiment, the siRNA for inhibiting MYPT1 expression from MYPT1 mRNA (siMYPT1) comprises the following nucleic acid sequence: 5′-CGACAUCAAUUACGCCAAUUU-3′. In yet another preferred embodiment, the siRNA for inhibiting MYPT1 expression from MYPT1 mRNA (siMYPT1) comprises the following nucleic acid sequence: 5′-UCGGCAAGGUGUUGAUAUAUU-3′. These siMYPT1 oligonulceotides may be used alone or in combination with each other. Using these oligonucleotides, expression of MYPT1 has been drastically reduced (see Example 10 and FIGS. 12 and 13).

Other suitable siRNA for MYPT1 can be obtained from the MYPT1 sequences available in the prior art and using the guidelines and examples provided herein (see Example 10 and FIG. 12).

Also contemplated herein are siRNAs directed against myosin phosphatase subunit M20. In a preferred embodiment of the present invention, the siRNA molecule has at least one strand that is substantially complementary to at least ten but no more than thirty consecutive nucleotides of an M20 nucleotide sequence. M20 nucleotide sequences are available at the web site of NCBI. Suitable siM20 siRNAs can be designed and tested using the guidance and assays described herein, e.g., below, Example 10 and FIG. 12.

In some embodiments of the present invention, it is desirable to knockdown expression of PP1α by, e.g., using an siRNA. The siRNA molecule has at least one strand that is substantially complementary to at least ten but no more than thirty consecutive nucleotides of a PP1α. In a preferred embodiment, the siRNA for inhibiting PP1α expression from PP1α mRNA (siPP1α) comprises the following nucleic acid sequence: 5′-GAACGUGCAGCUGACAGAG-3′. In another preferred embodiment, the siRNA for inhibiting PP1α expression from PP1α mRNA (siPP1α) comprises the following nucleic acid sequence: 5′-GGGCAAGUAUGGGCAGUUC-3′ In yet another preferred embodiment, the siRNA for inhibiting PP1α expression from PP1α mRNA (siPP1α) comprises the following nucleic acid sequence: 5′-GGUUGUAGAAGAUGGCUAU-3′. Other useful siRNAs for inhibiting PP1α are 5′-GAACGUGCAGCUGACAGAGtt-3′,5′-GGGCAAGUAUGGGCAGUUCtt-3′ and 5′-GGUUGUAGAAGAUGGCUAUtt-3′. The siPP1α oligonucleotides may be used alone or in combination with each other to inhibit PP1α expression. Using these oligonucleotides, expression of PP1α has been drastically reduced (see Example 11; FIG. 13).

In other embodiments of the present invention, it is desirable to knockdown expression of PP1γ by, e.g., using an siRNA. The siRNA molecule has at least one strand that is substantially complementary to at least ten but no more than thirty consecutive nucleotides of a PP1γ. In a preferred embodiment, the siRNA for inhibiting PP1γ expression from PP1γ mRNA (siPP1γ) comprises the following nucleic acid sequence: 5′-CCGAUAAUGCUUUCUUUGG-3′. In another preferred embodiment, the siRNA for inhibiting PP1γ expression from PP1γ mRNA (siPP1γ) comprises the following nucleic acid sequence: 5′-GCAAGCCAAGCACUUCAUU-3′. In yet another preferred embodiment, the siRNA for inhibiting PP1γ expression from PP1γ mRNA (siPP1γ) comprises the following nucleic acid sequence: 5′-CGGGCAGUACUAUGAUUUG-3′. Other useful siRNAs for inhibiting PP1γ are 5′-CCGAUAAUGCUUUCUUUGGtt-3′,5′-GCAAGCCAAGCACUUCAUUtt-3′ and 5′-CGGGCAGUACUAUGAUUUGtt-3′. The siPP1γ oligonucleotides may be used alone or in combination with each other to inhibit PP1γ expression. Using these oligonucleotides, expression of PP1γ has been drastically reduced (Example 11; FIG. 13).

In another preferred embodiment, an siRNA molecule for inhibiting PP1α, PP1β, PP1γ, and MYPT1 includes a sequence that is at least 90% homologous, preferably 95%, 99%, or 100% homologous, to one of the following nucleic acid sequences: 5′-GAACGUGCAGCUGACAGAG-3′,5′-GGGCAAGUAUGGGCAGUUC-3′,5′-GGUUGUAGAAGAUGGCUAU-3′,5′-CCAGAAGCCAACUAUCUUU-3′,5′-GCCAACUAUCUUUUCUUAG-3′ or 5′-CGGAUAUGAAUUUUUUGCU-3′,5′-CCGAUAAUGCUUUCUUUGG-3′,5′-GCAAGCCAAGCACUUCAUU-3′,5′-CGGGCAGUACUAUGAUUUG-3′,5′-GAACGAGACUUGCGUAUGUUU-3′,5′-AAGAAUAGUUCGAUCAAUGUU-3′,5′-CGACAUCAAUUACGCCAAUUU-3′,5′-UCGGCAAGGUGUUGAUAUAUU-3′,5′-GAACGUGCAGCUGACAGAGtt-3′,5′-GGGCAAGUAUGGGCAGUUCtt-3′,5′-GGUUGUAGAAGAUGGCUAUtt-3′,5′-CCAGAAGCCAACUAUCUUUtt-3′,5′-GCCAACUAUCUUUUCUUAGtt-3′,5′-CGGAUAUGAAUUUUUUGCUtt-3′,5′-CCGAUAAUGCUUUCUUUGGtt-3′,5′-GCAAGCCAAGCACUUCAUUtt-3′ and 5′-CGGGCAGUACUAUGAUUUGtt-3′. Without undue experimentation and using the disclosure of this invention, it is understood that additional siRNAs for inhibiting PP1α, PP1β, PP1γ, and MYPT1 that modulate myosin phosphatase expression can be designed and used to practice the methods of the invention.

A preferable siRNA used in the present invention has the general formula:

5′-[A]-[B]-[A′]-3′

wherein [A] is a ribonucleotide sequence corresponding to a target sequence of a PP1α, PP1γ, PP1γ, MYPT1, or M20 gene; [B] is a ribonucleotide sequence consisting of about 3 to about 23 nucleotides; and [A′] is a ribonucleotide sequence complementary to [A]. Herein, the phrase a “target sequence of a PP1α, PP1β, PP1γ, MYPT1, or M20 gene” refers to a sequence that, when introduced into a mammalian cell, is effective for inhibiting or reducing the translation of a PP1α, PP1β, PP1γ, MYPT1, or M20 mRNA.

Other than the siRNAs disclosed herein, siRNAs useful to practice a method of the present invention can be identified as follows. Beginning with the AUG start codon of the transcript (e.g., a PP1α, PP1β, PP1γ, MYPT1, or M20 mRNA), the transcript is scanned downstream for AA dinucleotide sequences. The occurrence of each AA and the 3′ adjacent 19 nucleotides as potential siRNA target sites are recorded. It may not be recommended to design siRNA against the 5′ and 3′ untranslated regions (UTRs) and regions near the start codon (within 75 bases) as these may be richer in regulatory protein binding sites, and thus the complex of endonuclease and siRNAs that are designed against these regions may interfere with the binding of UTR-binding proteins and/or translation initiation complexes (Tuschl, et al. 1999, Genes Dev 13(24):3191-7). Then the potential target sites are compared to the human genome database. Any target sequences with significant homology to other coding sequences are eliminated from consideration. The homology search can be performed using BLAST (Altschul et. al., 1997, Nucleic Acids Res 25:3389-402; Altschul et. al., 1990, J Mol Biol 215:403-10). Next, qualifying target sequences are selected for synthesis. On the website of Ambion, several preferable target sequences can be selected along the length of the gene for evaluation.

The double-stranded molecule of the present invention comprises a sense strand and an antisense strand, wherein the sense strand comprises a ribonucleotide sequence corresponding to a PP1α, PP1β, PP1γ, MYPT1 or M20 target sequence, and wherein the antisense strand comprises a ribonucleotide sequence which is complementary to said sense strand, wherein said sense strand and said antisense strand hybridize to each other to form said double-stranded molecule, and wherein said double-stranded molecule, when introduced into a cell expressing a PP1α, PP1β, PP1γ, MYPT1 or M20 gene, inhibits expression of said gene.

The double-stranded molecule of the present invention may be a polynucleotide derived from its original environment (i.e., when it is a naturally occurring molecule, the natural environment), physically or chemically altered from its natural state, or chemically synthesized. According to the present invention, such double-stranded molecules include those composed of DNA, RNA, and derivatives thereof. A DNA is suitably composed of bases such as A, T, C and G, and T is replaced by U in an RNA.

SiRNAs may be expressed from a vector. The vector preferably comprises a regulatory sequence adjacent to the region encoding the present double-stranded molecule that directs the expression of the molecule in a cell. For example, the double-stranded molecules of the present invention are intracellularly transcribed by cloning their coding sequence into a vector containing, e.g., a RNA polymerase III transcription unit from the small nuclear RNA (snRNA) U6 or the human H1 RNA promoter.

Alternatively, the present vectors are produced, for example, by cloning the target sequence into an expression vector so the objective sequence is operatively-linked to a regulatory sequence of the vector in a manner to allow expression thereof (transcription of the DNA molecule) (Lee et al., 2002, Nature Biotechnology 20:500-505). For example, the transcription of an RNA molecule having an antisense sequence to the target sequence is driven by a first promoter (e.g., a promoter sequence linked to the 3′-end of the cloned DNA) and that having the sense strand to the target sequence by a second promoter (e.g., a promoter sequence linked to the 5′-end of the cloned DNA). The expressed sense and antisense strands hybridize to each other in vivo to generate an siRNA construct to silence a gene that comprises the target sequence. Furthermore, two constructs (vectors) may be utilized to respectively produce the sense and anti-sense strands of an siRNA construct.

For introducing the vectors into a cell, transfection-enhancing agent can be used. FuGENE6® (Roche Diagnostic), Lipofectamine® 2000 (Invitrogen), Oligofectamine® (Invitrogen), and Nucleofector® (Wako pure Chemical) are useful as the transfection-enhancing agent. Transfection of vectors expressing siRNA polynucleotides of the invention can be used to inhibit a myosin phosphatase in a mammalian cell. Thus, it is another aspect of the present invention to provide a double-stranded molecule comprising a sense-strand and antisense-strand which molecule functions as an siRNA for PP1α, PP1β, PP1γ, MYPT1 or M20, and a vector encoding the double-stranded molecule.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In a preferred embodiment, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. application publication number 20040019001 and U.S. Pat. No. 6,673,611 (incorporated by reference). Collectively, all such altered RNAs described above are referred to as modified siRNAs.

Preferably, RNAi is capable of decreasing the expression of myosin phosphatase in a cell by at least 10%, 20%, 30%, or 40%, more preferably by at least 50%, 60%, or 70%, and most preferably by at least 75%, 80%, 90%, 95% or more.

Introduction of siRNA into cells can be achieved by methods known in the art and disclosed herein, including for example, microinjection, electroporation, or transfection of a vector comprising a nucleic acid from which the siRNA can be transcribed. Alternatively, an siRNA for myosin phosphatase can be directly introduced into a cell in a form that is capable of binding to myosin phosphatase mRNA transcripts. To increase durability and membrane-permeability the siRNA may be combined or modified with liposomes, poly-L-lysine, lipids, cholesterol, lipofectine or derivatives thereof. Preferred are cholesterol-conjugated siRNA for myosin phosphatase (see, Song et al., Nature Med. 9:347-351 (2003)).

SiRNAs and vectors comprising siRNA nucleic acid sequences and methods for preparing and using same are described, for example, in U.S. application publication number. 20060051815, which is incorporated herewith in its entirety by reference.

B. Small Molecules

A variety of compounds can be used to inhibit the level or activity of a myosin phosphatase and in particular inhibit the dephosphorylation of a histone deacetylase by the myosin phosphatase or inhibit the interaction between a histone deacetylase and the myosin phosphatase. In a preferred embodiment the inhibitor is a small molecule compound which can be identified as described herein.

C. Antisense RNA And Ribozymes

A variety of compounds can be used to inhibit the level or activity of a myosin phosphatase and in particular inhibit the dephosphorylation of a histone deacetylase by the myosin phosphatase or inhibit the interaction between a histone deacetylase and the myosin phosphatase. For example, the expression of myosin phosphatase or the expression of a subunit thereof, such as PP1β, MYPT1, or M20 can be inhibited by administering to a cell or a subject a nucleic acid that inhibits or antagonizes the expression of a PP1β, MYPT1, or M20 gene. In addition to siRNAs, described above, antisense oligonucleotides or ribozymes which disrupt the expression of a PP1β, MYPT1, or M20 gene can be used for modulating the level or activity of a myosin phosphatase. In a preferred embodiment the inhibitor is an anti-sense RNA, which can be identified as described herein.

As noted above, antisense-oligonucleotides corresponding to any of the nucleotide sequence of a PP1α, PP1β, PP1γ, MYPT1, or M20 gene can be used to reduce the expression level of the respective gene. Specifically, the antisense-oligonucleotides against the PP1α, PP1β, PP1γ, MYPT1, or M20 genes may act by binding to any of the corresponding mRNAs, thereby inhibiting the transcription or translation of these genes, promoting the degradation of the mRNAs, and/or inhibiting the expression of proteins encoded by the PP1α, PP1β, PP1γ, MYPT1, or M20 genes, and finally inhibiting the function of the proteins.

Anti-sense oligonucleotides and siRNAs of the invention can also be defined by their ability to hybridize specifically to mRNA or cDNA from the genes disclosed herein.

An antisense-oligonucleotide and derivatives thereof can be made into an external preparation, such as a liniment or a poultice, by mixing with a suitable base material which is inactive against the derivative.

The antisense-oligonucleotides of the invention inhibit the expression of at least one protein encoded by a PP1α, PP1β, PP1γ, MYPT1 or M20 gene, and thus are useful for suppressing the biological activity of a myosin phosphatase.

The nucleic acids that inhibit one or more gene products of over-expressed genes also include ribozymes against one or more of the PP1α, PP1β, PP1γ, MYPT1 or M20 gene(s). The ribozymes inhibit the expression of PP1α, PP1β, PP1γ, MYPT1 or M20 proteins and are thereby useful for suppressing the biological activity of the myosin phosphatase. Therefore, a composition comprising the ribozyme is useful in a method for inducing apoptosis in a mammalian cell or in a method for the treatment of a pathological condition as described herein.

Generally, ribozymes are classified into large ribozymes and small ribozymes. A large ribozyme is known as an enzyme that cleaves the phosphate ester bond of nucleic acids. After the reaction with the large ribozyme, the reacted site consists of a 5′-phosphate and 3′-hydroxyl group. The large ribozyme is further classified into (1) group I intron RNA catalyzing transesterification at the 5′-splice site by guanosine; (2) group II intron RNA catalyzing self-splicing through a two step reaction via lariat structure; and (3) RNA component of the ribonuclease P that cleaves the tRNA precursor at the 5′ site through hydrolysis. On the other hand, small ribozymes have a smaller size (about 40 bp) compared to the large ribozymes and cleave RNAs to generate a 5′-hydroxyl group and a 2′-3′ cyclic phosphate. Hammerhead type ribozymes (Koizumi et al., 1988, FEBS Lett. 228:225) and hairpin type ribozymes (Buzayan, 1986, Nature 323:349; Kikuchi and Sasaki, 1991, Nucleic Acids Res. 19: 6751) are included in the small ribozymes. Methods for designing and constructing ribozymes are known in the art (see Koizumi et al., 1988, FEBS Lett. 228:225; Koizumi et al., 1989, Nucleic Acids Res. 17:7059; Kikuchi and Sasaki, 1991, Nucleic Acids Res. 19: 6751) and ribozymes inhibiting the expression of an PP1β, MYPT1 or M20 protein can be constructed based on the sequence information of the nucleotide sequence encoding a PP1β, MYPT1 or M20 protein according to conventional methods for producing ribozymes.

D. Dominant Negative Proteins

A variety of compounds can be used to inhibit the level or activity of a myosin phosphatase and in particular inhibit the dephosphorylation of a histone deacetylase by the myosin phosphatase or inhibit the interaction between a histone deacetylase and the myosin phosphatase. In a preferred embodiment the inhibitor is a dominant negative protein which can be identified as described herein.

In a preferred embodiment, a dominant negative protein inhibiting the level or activity of a myosin phosphatase is the protein kinase C-potentiated inhibitor protein 17 kDa (CPI-17). Thus, CPI-17 or an active fragment thereof can be used as an inhibitor of myosin phosphatase to practice the methods of the present invention.

Other dominant negative proteins for myosin phosphatase may be identified using methods known in the art and the assays disclosed herein.

When the candidate compound is a protein, the amino acid sequence of the protein is determined, an oligo DNA is synthesized based on the sequence, and cDNA libraries are screened using the oligo DNA as a probe to obtain a DNA encoding the protein.

### III. Activators of Myosin Phosphatase

As described herein, dephosphorylation of a histone deacetylase, such as HDAC7, leads to re-entry of HDAC7 into the nucleus and to the inhibition of apoptosis. Thus, compounds that modulate the level or activity of a myosin phosphatase, are useful for controlling the subcellular localization of HDAC7 and for inhibiting, reducing or inducing apoptosis. Compounds that increase the level or activity of a myosin phosphatase are particularly useful for promoting the re-entry of non-phosphorylated HDAC7 into the nucleus and subsequently inhibiting or reducing apoptosis. Such compounds can be identified as described herein. Preferably an activator of myosin phosphatase is a small molecule.

In one embodiment of the present invention, an activator of myosin phosphatase increases the enzymatic activity of myosin phosphatase. An increase of myosin phosphatase enzymatic activity is determined by comparing the enzymatic activity of the myosin phosphatase in the presence of a candidate agent to the enzymatic activity of the myosin phosphatase in the absence of such candidate agent. A higher enzymatic activity of the myosin phosphatase in the presence of a candidate agent compared to the enzymatic activity of the myosin phosphatase in the absence of such candidate agent indicates that the candidate agent is an activator of myosin phosphatase, in particular an activator of the enzymatic activity of the myosin phosphatase.

In another embodiment of the present invention, an activator of myosin phosphatase increases the expression of myosin phosphatase. An increase of myosin phosphatase expression is determined by comparing the level of myosin phosphatase polypeptide or myosin phosphatase mRNA in a first cell in the presence of a candidate agent to the level of myosin phosphatase polypeptide or myosin phosphatase mRNA in a second cell in the absence of the candidate agent. A higher level of the myosin phosphatase in the presence of the candidate agent compared to the level of the myosin phosphatase in the absence of such candidate agent indicates that the candidate agent is an activator of myosin phosphatase, in particular an activator of the myosin phosphatase expression.

Applicants have shown herein that there is an intricate balance between the enzymatic activity of myosin phosphatase dephosphorylating HDAC7 and PKD1 phosphorylating HDAC7. Thus, in yet another embodiment of the present invention, an activator of myosin phosphatase is an inhibitor of PKD1.

### IV. Identification of Inhibitors and Activators of Myosin Phosphatase

Inhibitors and activators, referred to herein as modulators of level or activity of myosin phosphatase are identified using methods known in the art and described herein. A number of different screening protocols can be utilized to identify agents that modulate the level of expression or activity of a myosin phosphatase. The term “modulate” encompasses an increase or a decrease in the measured activity of a myosin phosphatase or histone deacetylase when compared to a suitable control.

These screening protocols can be used in cells, particularly mammalian cells, and especially human cells. Alternatively, as further described herein, screening assays can be performed in vitro. In vitro screening assays may use (i) a naturally occurring histone deacetylase and a naturally occurring myosin phosophatase, (ii) a recombinantly produced histone deacetylase and a recombinantly produced myosin phosophatase, or (iii) combinations of naturally occurring and recombinantly produced polypeptides.

In general terms, the screening methods involve screening a variety of agents to identify an agent that modulats the level of expression or activity of a myosin phosphatase. The method generally comprises the step of (a) contacting a candidate compound with a myosin phosphatase, with a biological sample comprising a myosin phosphatase or with a mammalian cell expressing a myosin phosphatase; and (b) assaying an activity of the myosin phosphatase in the presence of the candidate compound. An increase or a decrease in the activity measured in comparison to the activity of the myosin phosphatase in a suitable control (e.g., a myosin phosphatase in the absence of the candidate compound, a biological sample comprising a myosin phosphatase in the absence of the candidate compound or a mammalian cell expressing a myosin phosphatase in the absence of the candidate compound) is an indication that the candidate compound modulates an activity of the myosin phosphatase. Once a candidate compound or candidate agent has been identified in one of the screening methods of the present invention, it is typically referred to as a compound or agent, rather than a candidate compound or candidate agent.

Agents that increase or decrease an HDAC activity of a polypeptide to the desired extent may be selected for further study, and assessed for cellular availability, cytotoxicity, biocompatibility, etc.

In one aspect, the screening methods involve screening candidate agents to identify an agent that inhibits or reduces the level of expression or activity of a myosin phosphatase. In another aspect, the screening methods involve screening candidate agents to identify an agent that increases the level of expression or activity of a myosin phosphatase.

In a first aspect, the present invention provides a method for identifying a candidate compound which modulates the dephosphorylation of a histone deacetylase by a myosin phosphatase. In a preferred embodiment, this method comprises the steps of (a) assaying for the dephosphorylation of a histone deacetylase by a myosin phosphatase, which is able to dephosphorylate the histone deacetylase, and (b) assaying for the dephosphorylation in the presence of a candidate compound, to identify a candidate compound which modulates the dephosphorylation.

In another embodiment, the screening assay comprises the step of performing a first assay determining the dephosphorylation of a histone deacetylase by a myosin phosphatase and performing a second assay determining the dephosphorylation of the histone deacetylase by the myosin phosphatase in the presence of a candidate compound for modulating the dephosphorylation of the histone deacetylase by the myosin phosphatase. Prefereably, the first assay and the second assay are performed under similar or identical conditions, differeing only by the absence or presence of the candidate compound. Typically it is sufficient to perform the first assay in the absence of the candidate compound once and then perform the second assay in the presence of different candidate compounds as often as different compounds are tested. The result of the first assay is compared to the result(s) of the second assay(s). A difference in a result of the first assay when compared to the result(s) of the second assay(s) indicates that the candidate compound which was tested is a compound which modulates the dephosphorylation of the histone deacetylase by the myosin phosphatase.

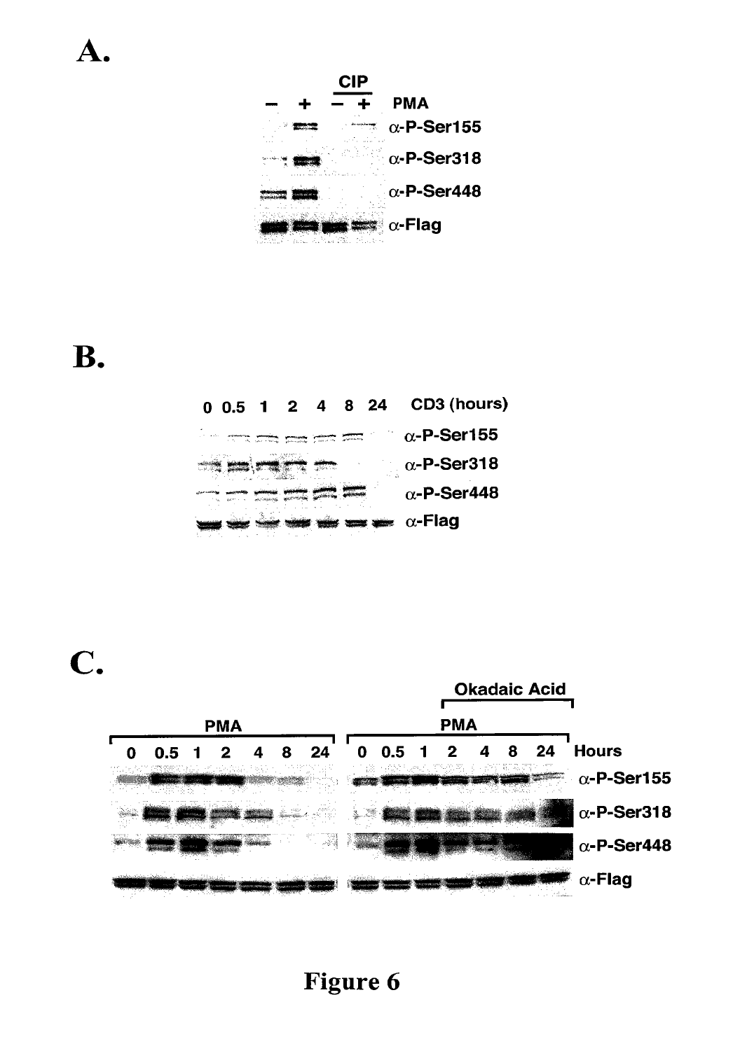

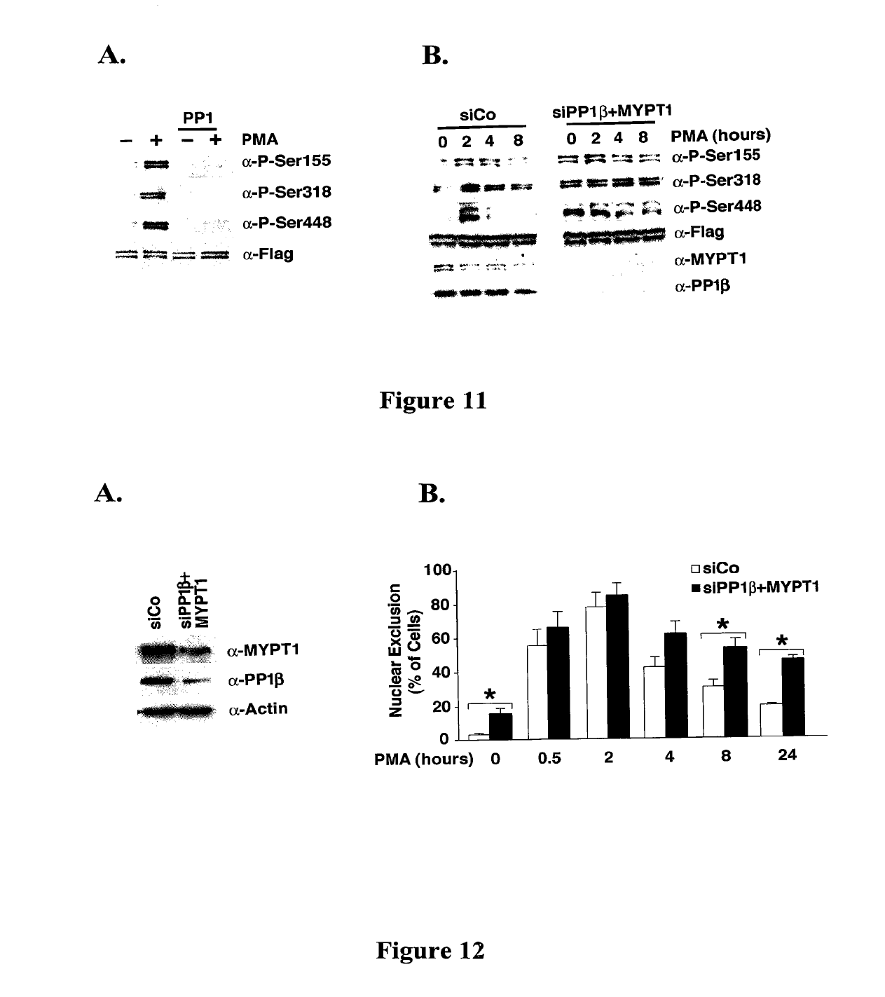

Using antibodies against mouse HDAC7 phosphorylated at serine residues 178, 344 and 479 or human HDAC7 phosphorylated at amino acid positions 155, 318, and 448 (see, e.g., Examples 4 and 9; FIGS. 6 and 11), the modulation of dephosphorylation of a histone deacetylase, and in particular HDAC7, by myosin phosphatase in the absence or presence of an candidate compound, can be assessed.

Using an HDAC7-GFP protein to monitor the subcellular localization of an HDAC7 as described herein can be used to identify a modulator of myosin phosphatase activity. An activator of myosin phosphatase will cause the HDAC7 to remain in the nucleus and/or promote the re-entry of HDAC7 into the nucleus.

Also provided herein is a method for identifying a candidate compound which modulates the interaction between a histone deacetylase and a myosin phosphatase. In a preferred embodiment of the present invention, this method comprises the steps of (a) assaying for the interaction between a histone deacetylase and a myosin phosphatase, which is able to bind to the histone deacetylase, and (b) assaying for the interaction in the presence of a candidate compound, to identify a candidate compound which modulates the interaction.

In another embodiment, this screening assay comprises the step of performing a first assay determining the interaction between a histone deacetylase and a myosin phosphatase and performing a second assay determining the interaction between the histone deacetylase and the myosin phosphatase in the presence of a candidate compound for modulating the interaction between the histone deacetylase and the myosin phosphatase. Prefereably, the first assay and the second assay are performed under similar or identical conditions, differeing only by the absence or presence of the candidate compound. Typically it is sufficient to perform the first assay in the absence of the candidate compound once and then perform the second assay in the presence of different candidate compounds as often as different compounds are tested. The result of the first assay is compared to the result(s) of the second assay(s). A difference in a result of the first assay when compared to the result(s) of the second assay(s) indicates that the candidate compound which was tested is a compound which modulates the interaction between the histone deacetylase and the myosin phosphatase.

Using, e.g., the binding assays described herein, such as an immunoprecipitation assay, (see, e.g., Examples 7, 8, and 9; FIGS. 9, 10, and 11), the modulation of binding of a histone deacetylase, and in particular HDAC7, to a myosin phosphatase in the absence or presence of an candidate compound, can be assessed.

In another aspect of the present invention, a method for identifying a candidate compound capable of reducing or inhibiting apoptosis in a mammalian cell expressing a histone deacetylase, preferably a class II histone deacetylase, is provided. In a preferred embodiment of the present invention, this method comprises the steps of (a) assaying expression of a gene regulated in a mammalian cell by the histone deacetylase and a MEF2 family protein, (b) contacting the mammalian cell with a candidate compound, and (c) determining whether, in the presence of the candidate compound, the expression of the gene regulated by the histone deacetylase and the MEF2 family protein is inhibited, wherein if the expression of the gene in the presence of the candidate compound is inhibited, the candidate compound is capable of reducing or inhibiting apoptosis.

In a preferred embodiment of the present invention, the MEF2 family protein is the transcription factor MEF2-D.

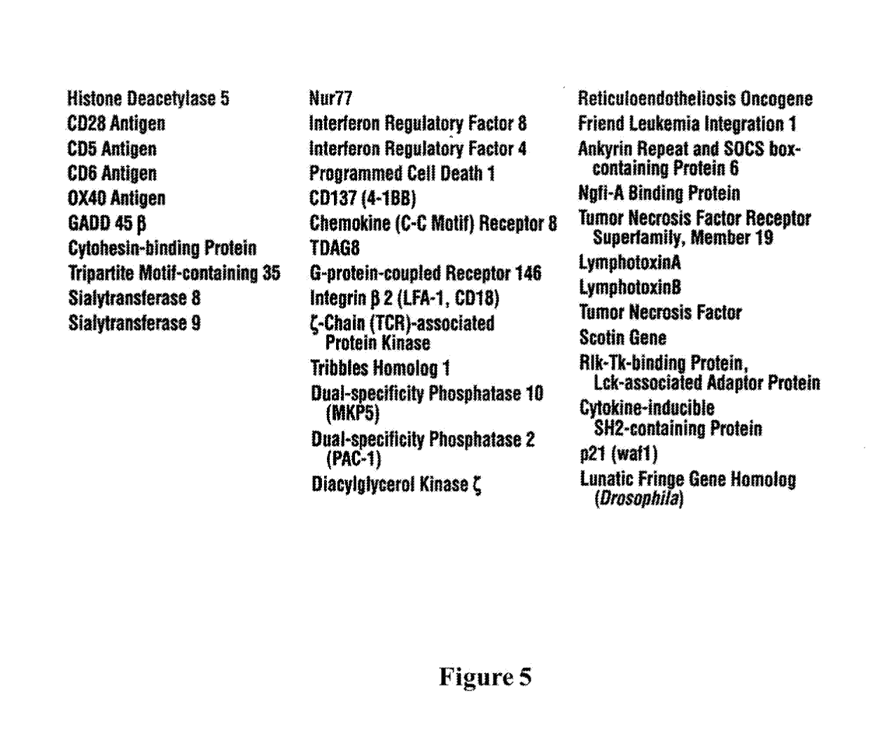

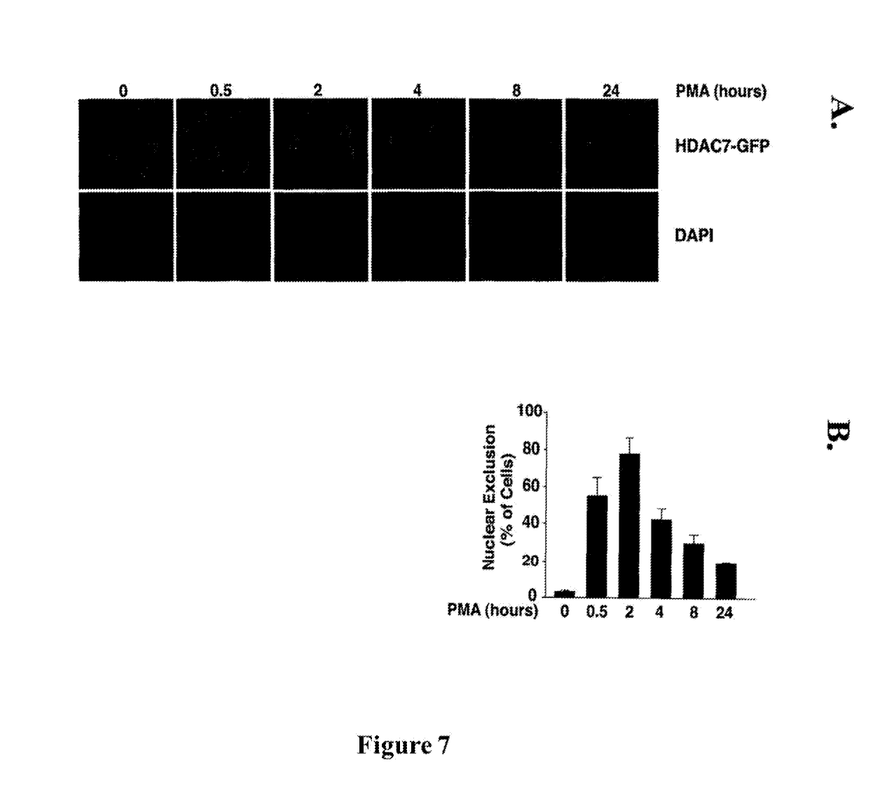

As described herein, histone deacetylases, and in particular HDAC7 regulate the expression of a variety of genes. A representative set of genes of which the expression is regulated by HDAC7 is shown in FIG. 5. Thus, in a preferred embodiment of the present invention, a gene regulated by a histone deacetylase, in particular by HDAC7, and a MEF2 family protein is selected from the group of genes shown in FIG. 5. A preferred gene regulated by a histone deacetylase, in particular by HDAC7, and a MEF2 family protein is Nur77. Nur77 expression in the absence or presence of a candidate compound can be determined using assays described herein, for example, Northern blot analysis, in situ hybridization for Nur77 RNA detection or by Western blot analysis as shown in FIG. 7.

As described herein, myosin phosphatase dephosphorylation of histone deacetylase, and in particular HDAC7, leads to a reduction or inhibition of apoptosis. Thus, in yet another aspect of the present invention, a method for identifying a candidate compound for reducing or inhibiting apoptosis is provided. In a preferred embodiment of this method, the method comprises the steps of (a) contacting a myosin phosphatase with a candidate compound, and (b) determining whether the candidate compound binds to the myosin phosphatase, increases the activity of the myosin phosphatase, or increases binding of the myosin phosphatase to a histone deacetylase, wherein a candidate compound that binds to the myosin phosphatase, increases the activity of the myosin phosphatase, or increases binding of the myosin phosphatase to the histone deacetylase is a candidate compound useful for reducing or inhibiting apoptosis.

Optionally, the methods for identifying a candidate compound as described herein, comprise the step of selecting the compound that binds to a myosin phosphatase or modulates the level or activity of the myosin phosphatase.

The present invention also provides methods for inducing apoptosis. In a preferred embodiment of this invention, this method comprises the steps of (a) contacting a myosin phosphatase with a candidate compound, and (b) determining whether the candidate compound binds to the myosin phosphatase, inhibits the activity of the myosin phosphatase, or inhibits binding of the myosin phosphatase to a histone deacetylase, wherein a candidate compound that binds to the myosin phosphatase, inhibits the activity of the myosin phosphatase, or inhibits binding of the myosin phosphatase to the histone deacetylase is a candidate compound useful for inducing apoptosis.

Candidate compounds useful for reducing, inhibiting or inducing apoptosis identified by the method described herein can be assessed by using the apoptosis assay described herein (see FIG. 12) and assays known in the art.

In another aspect, the present invention provides a method for identifying a candidate compound which mimics the effect of a myosin phosphatase. In a preferred embodiment of the present invention, this method comprises the steps of (a) assaying the enzymatic activity or binding activity of a histone deacetylase in the presence of a myosin phosphatase, (b) contacting the histone deacetylase with a compound, and (c) determining whether, in the presence of the compound, the histone deacetylase mimics the enzymatic activity or binding activity of the histone deacetylase in the presence of the myosin phosphatase; wherein if the histone deacetylase mimics the enzymatic activity or binding activity of the myosin phosphatase, the candidate compound mimics the effect of the myosin phosphatase.

Candidate compounds which mimic the effect of a myosin phosphatase can be tested using the assays described herein, such as Northern blot assays, in situ hybridization, Western blot assays, immunoprecipitation assays, apoptosis assays, and the like.

A preferred histone deacetylase for practicing the methods and compositions of the present invention is HDAC7. HDAC7 is a class IIa histone deacetylase. Other class IIa histone deacetylases share significant homology to HDAC7, in particular in a protein region comprising the serine residues that are phosphorylated upon TCR activation and which become dephosphorylated by myosin phosphatase. Thus, other preferred histone deacetylases include, but are not limited to class IIa histone deacetylases HDAC4, HDAC5, and HDAC9.

A candidate compound is assessed for any cytotoxic activity it may exhibit toward the cell used in the assay, using well-known assays, such as trypan blue dye exclusion, an MTT ([3-(4,5-dimethylthiazol-2-yl)-2,5-d-iphenyl-2H-tetrazolium bromide]) assay, and the like. Agents that do not exhibit cytotoxic activity are considered candidate agents.

A. Screening for Compounds

In addition to the screening methods described above, in another embodiment of the screening method of the present invention, a two-hybrid system utilizing cells may be used (“MATCHMAKER® Two-Hybrid system”, “Mammalian MATCHMAKER® Two-Hybrid Assay Kit”, “MATCHMAKER® one-Hybrid system” (Clontech); “HybriZAP® Two-Hybrid Vector System” (Stratagene); see also Dalton and Treisman, 1992, Cell 68: 597-612; Fields and Sternglanz, 1994, Trends Genet. 10:286-92).

In the two-hybrid system, for example, a histone deacetylase, preferable, an HDAC7 polypeptide, is fused to the SRF-binding region or GAL4-binding region and expressed in yeast cells. Myosin phosphatase or a subunit of myosin phosphatase that binds to the histone deacetylase polypeptide is fused to the VP 16 or GAL4 transcriptional activation region and also expressed in the yeast cells in the existence of a test compound. Alternatively, myosin phosphatase or a subunit of myosin phosphatase that binds to the histone deacetylase polypeptide may be fused to the SRF-binding region or GAL4-binding region, and the histone deacetylase polypeptide to the VP 16 or GAL4 transcriptional activation region. When the test compound does not inhibit the binding between histone deacetylase and myosin phosphatase or a subunit of myosin phosphatase that binds to the histone deacetylase polypeptide, the binding of the two activates a reporter gene, making positive clones detectable. As a reporter gene, for example, HIS3 gene, Ade2 gene, lacZ gene, CAT gene, luciferase gene can be used.

Any test compound, for example, cell extracts, cell culture supernatants, products of fermenting microorganisms, extracts from marine organism, plant extracts, purified or crude proteins, peptides, non-peptide compounds, synthetic micromolecular compounds and natural compounds can be used in the screening methods of the present invention. The test compound of the present invention can also be obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection are limited to peptide libraries, while the other four approaches are applicable to peptide, non-peptide oligomer or small molecule libraries of compounds (Lam, 1997, Anticancer Drug Des. 12:145). Examples of methods for the synthesis of molecular libraries can be found in the art (DeWitt et al., 1993, Proc Natl Acad Sci USA 90: 6909; Erb et al., 1994, Pro. Natl Acad Sci USA 91:11422; Zuckermann et al., 1994, J Med Chem 37:2678; Cho et al., 1993, Science 261:1303; Carell et al., 1994, Angew Chem. Int. Ed Engl. 33:2059; Carell et al., 1994, Angew Chem Int Ed. Engl. 33:2061; Gallop et al., 1994, J Med Chem 37:1233). Libraries of compounds may be presented in solution (see Houghten, 1992, Bio/Techniques 13:412) or on beads (Lam, 1991, Nature 354: 82), chips (Fodor, 1993, Nature 364:555), bacteria (U.S. Pat. No. 5,223,409), spores (U.S. Pat. Nos. 5,571,698; 5,403,484, and 5,223,409), plasmids (Cull et al., 1992, Proc Natl Acad Sci USA 89:1865) or phage (Scott and Smith, 1990, Science 249:386; Devlin, 1990, Science 249:404; Cwirla et al., 1990, Proc Natl Acad Sci USA 87:6378; Felici, 1991, J Mol Biol 222 301; US Pat. Application 20020103360). The test compound exposed to a cell or protein according to the screening methods of the present invention may be a single compound or a combination of compounds. When a combination of compounds is used in the screening methods of the invention, the compounds may be contacted sequentially or simultaneously.

A compound isolated by the screening methods of the present invention is a candidate for a drug which modulates the level or activity of a myosin phosphatase, for reducing, inhibiting, or inducing apoptosis and for treating or preventing a pathological condition as described herein. A compound in which a part of the structure of the compound obtained by the present screening methods of the present invention is converted by addition, deletion and/or replacement, is included in the compounds obtained by the screening methods of the present invention. A compound effective in suppressing the expression of over-expressed genes, i.e., one or more of those listed in FIG. 5, is deemed to have a clinical benefit and can be further tested for its ability to treat or prevent a disorder, disease or pathological condition in animal models or test subjects.

Both naturally occurring histone deacetylase and myosin phosphatase poly peptides and recombinant histone deacetylase and myosin phosphatase poly peptides can be used to practice the methods of the present invention.

### V. Testing Inhibitors and Activators of Myosin Phosphatase

Methods for testing and assaying compounds, agents or antagonists identified by methods described herein, are provided herein and involve a variety of accepted tests to determine whether a given candidate compound, agent, or small molecule is useful to practice a method of the present invention. Methods of the present invention may optionally comprise the step of detecting a nucleic acid, such as an mRNA or a polypeptide. In one embodiment, such a method comprises determining or detecting an mRNA, preferably a myosin phosphatase (PP1β, MYPT1, or M20) mRNA. Other mRNAs, such as a histone deacetylase mRNA, in particular an HDAC7 mRNA, a Nur77 mRNA, or an mRNA of any gene shown in FIG. 5, and other mRNAs encoding polypeptides described herein can also be determined using the following methods.

A. Detection of mRNAs

Methods of evaluating mRNA expression of a particular gene are well known to those of skill in the art, and include, inter alia, hybridization and amplification based assays.

1. Direct Hybridization-Based Assays

Methods of detecting and/or quantifying the level of a gene transcript (mRNA or cDNA made therefrom) using nucleic acid hybridization techniques are known to those of skill in the art. For example, one method for evaluating the presence, absence, or quantity of a polynucleotide involves a Northern blot. Gene expression levels can also be analyzed by techniques known in the art, e.g., dot blotting, in situ hybridization, RNase protection, probing DNA microchip arrays, and the like (e.g., see Sambrook, J., Fritsch, E. F., and Maniatis, “Molecular Cloning A Laboratory Manual” by T. published by Cold Spring Harbor Laboratory Press, 2nd edition, 1989).

2. Amplification-Based Assays

In another embodiment, amplification-based assays are used to measure the expression level of a gene. In such an assay, the nucleic acid sequences act as a template in an amplification reaction (e.g., Polymerase Chain Reaction, or PCR). In a quantitative amplification, the amount of amplification product will be proportional to the amount of template in the original sample. Comparison to appropriate controls provides a measure of the level of an mRNA in the sample. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.). Exemplary methods using HDAC nucleic acids as a template for PCR and nucleic acid primers for RT-PCR are described herein (Example 1).

In one embodiment, a TaqMan based assay is used to quantify a polynucleotide. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, Heid et al., 1996, Genome Res 6(10):986-94; Morris et al., 1996, J Clin Microbiol 34(12):2933-6).

Other suitable amplification methods include, but are not limited to, ligase chain reaction (LCR) (see, Wu and Wallace, 1989, Genomics 4:560; Landegren et al., 1988, Science 241:1077; and Barringer et al., 1990, Gene 89:117), transcription amplification (Kwoh et al., 1989, Proc Natl Acad Sci USA 86:1173), self-sustained sequence replication (Guatelli et al., 1990, Proc Nat Acad Sci USA 87: 1874), dot PCR, linker adapter PCR, and the like.

B. Detection of Polypeptides

Methods of the present invention described herein, may optionally comprise the step of determining or detecting a polypeptide, such as a histone deacetylase, a myosin phosphatase or a Nur77 polypeptide. Other polypeptides, such as those listed in FIG. 5 and others described herein can also be determined using the following methods.

Expression levels of a polypeptide may be determined by a variety of methods, including, but not limited to, affinity capture, mass spectrometry, traditional immunoassays and immunoprecipitation assays, PAGE, Western Blotting, or HPLC as further described herein (e.g., see FIGS. 6-13 and Examples 4-11), or as known by one of skill in the art.

Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

C. Detection Of Enzymatic Activity

In a preferred embodiment of the present invention, enzymatic activity of a myosin phosphatase is determined. As described herein (see FIG. 11 and Example 9), myosin phosphatase dephosphorylates histone deacetylases, including HDAC7. This assay can be used to assess dephosphorylation by myosin phosphatase in the absence or presence of a candidate compound.

D. Detection Of Subcellular Localization of HDAC7

As described herein, myosin phosphatase dephosphorylates HDAC7 which then re-enters the nucleus. Thus, in a preferred embodiment of the present invention, enzymatic activity of a myosin phosphatase is determined by monitoring or determining the subcellular localization of HDAC7 as described herein.

Applicants propose that myosin phosphatase also dephosphorylates other class II HDACs, such as HDAC4 and HDAC5, which may also influence the subcellular localization of HDAC4 and HDAC5. Therefore, the activity of myosin phosphatase can also be determined by monitoring or determining the subcellular localization of HDAC4 and HDAC5. Monitoring or determining the subcellular localization of HDAC4 and HDAC5 is performed similar as for HDAC7 (described herein), i.e., using GFP fusion proteins or specific antibodies for HDAC4 or HDAC5.

Inhibitors of HDAC7 nuclear export can be identified as follows. One first determines the nuclear localization of HDAC7 in a cell, e.g., using the HDAC7-GFP expression construct and assay as described herein in the absence of a candidate agent and in the absence of a stimulus. Next, the cell is exposed to a stimulus. A preferred stimulus is PMA stimulation. Another preferred stimulus is TCR activation. An amount of HDAC7 remaining in the nucleus after exposure to the stimulus is determined as described herein. This measurement provides a first amount of nuclear HDAC7. In a parallel assay, the cell is contacted with a candidate agent and then exposed to the stimulus in the presence of a candidate agent. A preferred candidate agent is a small molecule. The amount of HDAC7 remaining in the nucleus in the presence of the candidate agent and after the stimulus is then compared to the amount of HDAC7 remaining in the nucleus after exposure to stimulus alone, i.e., in the absence of the candidate agent. This measurement provides a second amount of nuclear HDAC7. A candidate agent leading to a higher second amount of HDAC7 when compared to the first amount of HDAC7 inhibits nuclear export of HDAC7.

Inhibitors of HDAC7 nuclear export can be identified in cells as described herein, but also in organs and living organisms.

### VI. Methods for Using Inhibitors and Activators of Myosin Phosphatase

The present invention provides (i) methods for reducing or inhibiting apoptosis, (ii) methods for inducing apoptosis, and methods for treatment of a pathological condition. These methods make use of compounds agents, and small molecules described herein and/or identified using one or more methods described herein.

Methods of the present invention can be practiced using any mammalian cell. A preferred mammalian cell is a human cell.

Methods of the present invention can be practiced in vitro and in vivo. In a preferred embodiment, a method for inducing, reducing or inhibiting apoptosis is performed with a human cell which is in a human.

A. Reducing Or Inhibiting Apoptosis

This invention provides methods for reducing or preventing apoptosis in a mammalian cell expressing a histone deacetylase and a myosin phosphatase. In a preferred embodiment of the present invention, the method comprises the step of contacting the mammalian cell with an effective amount of an agent that increases the level or activity of the myosin phosphatase in the mammalian cell.

Control samples (untreated with candidate agents) are assigned a relative activity value of 100%. The level or activity of the myosin phosphatase in the mammalian cell is increased by at least 10% relative to the untreated control, preferably by at least 30%, at least 50%, at least 100%, at least 200%, at least 300%, at least 400%, at least 500%, or at least 1,000-3,000 or more relative to an untreated control.

B. Inducing Apoptosis

This invention also provides methods for inducing apoptosis in a mammalian cell expressing a histone deacetylase and a myosin phosphatase. In a preferred embodiment of the present invention, the method comprises the step of contacting the mammalian cell with an effective amount of an agent that inhibits the level or activity of the myosin phosphatase in the mammalian cell.

As shown herein, using an inhibitor of the level or activity of the myosin phosphatase induces apoptosis. A preferred agent is an siRNA as fully described herein.

Apoptosis can be assayed using any known method and methods described herein. Assays can be conducted on cell populations or an individual cell and include morphological assays and biochemical assays. A non-limiting example of a method of determining the level of apoptosis in a cell population is TUNEL (TdT-mediated dUTP nick-end labeling) labeling of the 3′-OH free end of DNA fragments produced during apoptosis (Gavrieli et al., 1992, J Cell Biol 119:493). The TUNEL method consists of catalytically adding a nucleotide, which has been conjugated to a chromogen system or a to a fluorescent tag, to the 3′-OH end of a 180-bp (base pair) oligomer DNA fragments in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus). Another marker that is currently available is annexin, sold under the trademark APOPTESTJ®. This marker is used in the “Apoptosis Detection Kit,” which is also commercially available, e.g., from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both. Other methods of testing for apoptosis are known in the art and can be used, including, e.g., the method disclosed in U.S. Pat. No. 6,048,703.

C. Treatment Of Pathological Conditions

In one aspect of the present invention, a method for the treatment of a pathological condition which involves an aberrant expression of at least one gene, the expression of which is controlled by a histone deacetylase, preferably a class II histone deacetylase, and a transcription factor of the MEF2 family protein, is provided. In a preferred embodiment of the present invention, this method comprises the step of administering to a patient a therapeutically effective amount of an agent that reduces the interaction between the histone deacetylase and a myosin phosphatase, whereby the expression of at least one gene is increased, thereby treating the pathological condition.

In another embodiment of this method, a therapeutically effective amount of an agent that reduces the dephosphorylation of a histone deacetylase by a myosin phosphatase is administered to the patient.

In another aspect of the present invention, a method for the treatment of a pathological condition which involves an aberrant expression of at least one gene, the expression of which is controlled by a histone deacetylase, preferably a class II histone deacetylase, and a transcription factor of the MEF2 family protein, is provided. In a preferred embodiment of the present invention, this method comprises the step of administering to a patient a therapeutically effective amount of an agent that increases the interaction between the histone deacetylase and a myosin phosphatase, whereby the expression of at least one gene is reduced or decreased, thereby treating the pathological condition. A preferred gene regulated by a histone deacetylase, preferably HDAC7, and a transcription factor of the MEF2 family protein, is selected from the genes shown in FIG. 5 of this application. A preferred gene regulated by a histone deacetylase, preferably HDAC7, and a transcription factor of the MEF2 family protein, is Nur77.

In another embodiment of this method, a therapeutically effective amount of an agent that increases the dephosphorylation of a histone deacetylase by a myosin phosphatase is administered to the patient.

In another embodiment of this method, a therapeutically effective amount of an agent that inhibits the nuclear export of HDAC7 is administered to the patient.

In one embodiment of the present invention, a pathological condition is characterized by an increase of expression of at least one gene shown in FIG. 5.

In another embodiment of the present invention, a pathological condition is characterized by a decrease of expression of at least one gene shown in FIG. 5.

Other pathological conditions which can be treated or prevented using a method of the present invention include a smooth muscle cell disorder, cardiac hypertrophy, hypertension, and asthma as further described below.

Maximal inhibition of myosin phosphatase activity is not always necessary, or even desired, to achieve a therapeutic effect. Agents which decrease a myosin phosphatase activity of a polypeptide are useful in inducing apoptosis in cancerous cells, particular cancerous thymocytes, and thus may be useful in treating cancers.

Methods of reducing tumor growth, and methods of reducing subject myosin phosphatase activity or level of myosin phosphatase, generally comprise administering to an individual an agent that modulates the level or activity of a myosin phosphatase. Whether tumor cell growth is inhibited or reduced can be assessed by any means known in the art, including, but not limited to, measuring tumor size, determining whether tumor cells are proliferating, e.g., by using a ³H-incorporation assay and/or counting tumor cells.

In addition, because of its ability to regulate apoptosis, inappropriate expression of HDAC7 in any tumor could potentially contribute to the tumor phenotype. Accordingly, it is conceivable that HDAC7 overexpression could be associated with any tumor. Because HDAC7 is expressed during T cell development at a time when T cells learn to distinguish self from nonself (thymic negative selection) overexpression or inappropriate expression of HDAC7 could lead to selective dysregulation of the immune system as seen in autoimmune diseases or immune deficiencies. In the case of autoimmune diseases, inhibition of HDAC7 activity or expression might be useful in the treatment of diseases such as juvenile diabetes, multiple sclerosis, systemic lupus erythematosus, rheumatoid arthritis and other related disorders. As shown herein, myosin phosphatase binds to and dephosphorylates HDCA7. Thus, a compound or agent identified herein that modulates the activity of the myosin phosphatase also affects the activity of HDAC7, and other class IIa histone deacetylases. As such, a compound or agent identified herein is also useful to treat any of the above conditions.

1. Smooth Muscle Cell Disorder

Phosphorylation of smooth muscle and on muscle myosin II is implicated in many physiological phenomena, including smooth muscle contraction, cell motility and cytokinesis. A distinct phosphatase, termed myosin phosphatase, is responsible for dephosphorylation of the phosphorylated light chain (for review, see Ito et al., 2004, Mol Cell Biochem 259:197-209). Applicants have described herein that the myosin phosphatase also binds to and dephosphorylates HDAC7, a histone deacetylase involved in gene regulation.

In another aspect of the present invention methods and compositions for the treatment of a disorder associated with smooth muscle cell hyperactivity, are provided.

In a preferred embodiment of the present invention, the method of treating a disorder associated with smooth muscle cell hyperactivity comprises the step of administering to an individual in need thereof an effective amount of an agent that modulates the level or activity of a myosin phosphatase activity and/or the level or activity of a myosin phosphatase mRNA, in particular, the level or activity of a PP1β, MYPT1 or M20 mRNA, in a smooth muscle cell.

Smooth muscle disorders that are amenable to treatment with a method of the present invention include GI tract motility disorders, such as Hirschprung's disease, duodenal atresia, chronic intestinal pseudo-obstruction; hypertension; asthma; atherosclerosis; benign hyperplasia of the prostate; irritable bowel syndrome; erectile dysfunction; urinary urgency; myometrium hyperactivity; bladder hyperactivity; acute kidney dilation due to obstruction by urolithiasis; tendon fibrosis (e.g., Dupuytren's disease, Ledderhose disease, etc.); penile induration (La Peyronie disease); fibrosis in various tissues; and hypertrophic scars.

In one embodiment, the disorder associated with smooth muscle cell hyperactivity is selected from hypertension, asthma, atherosclerosis, myometrium hyperactivity, bladder hyperactivity, benign hyperplasia of the prostate, fibrosis, and hypertrophic scars.

In another embodiment of the present invention, the disorder associated with smooth muscle cell hyperactivity is a cancer. Cancers that can be treated using a subject method are cancers arising from smooth muscle cells. Cancerous cells or cancers that can be treated using a subject method include, but are not limited to, benign or malignant tumors originating either from smooth muscle cells or like cells from any organ or tissue, such as uterine tumors of stromal cell origin (e.g., uterine leiomyosarcoma); intestinal tumors of stromal cell origin (including gastrointestinal stromal tumor cells); vascular wall tumors (including leiomyomas and leiomyosarcomas); and tumors from any cell type with smooth muscle differentiation (e.g., uterine endometrial stroma sarcoma with smooth muscle differentiation); and the like.

2. Hypertension And Hypertrophy

Hypertension, or high blood pressure, is a generally symptomless condition characterized by abnormally high pressure in the arteries. It is an extremely common disorder, affecting approximately 30% of adults. Untreated hypertension increases the risk of stroke, aortic disease, coronary heart disease, heart failure and cardiac hypertrophy (enlargement of the heart).

Heart disease and its manifestations, including congestive heart failure and cardiac hypertrophy, present a major health risk in the Western world. Cardiac hypertrophy is an increase in the size of the heart reflecting a quantitative increase in cell size and mass (rather than cell number) as the result of any or a combination of neural, endocrine or mechanical stimuli. In humans, hypertrophy is the compensatory response of the myocardium (cardiac muscle) to increased work as a result of an increase in blood pressure or blood volume (hemodynamic overload). Cardiac hypertrophy could arise from hypertension, mechanical load, myocardial infarction, cardiac arrhythmias, endocrine disorders and genetic mutations in cardiac contractile protein genes. The cardiac hypertrophic response is a complex syndrome and the elucidation of the pathways leading to both cardiac hypertrophy and heart failure will be beneficial in the treatment of cardiovascular disease resulting from various stimuli.

A family of transcription factors, the myocyte enhancer factor-2 family (MEF2), is involved in cardiac hypertrophy. There are four members of the MEF2 family in vertebrates, referred to as MEF2A, -B, -C, and D. These transcription factors share homology in an N-terminal MADS-box and an adjacent motif known as the MEF2 domain (see Olson et al., 1995, Dev Biol 172(1):2-14). Together, these regions mediate DNA binding, homo- and heterodimerization, and interaction with various cofactors. MEF2 binding sites are found in the control regions of the majority of skeletal, cardiac, and smooth muscle genes.

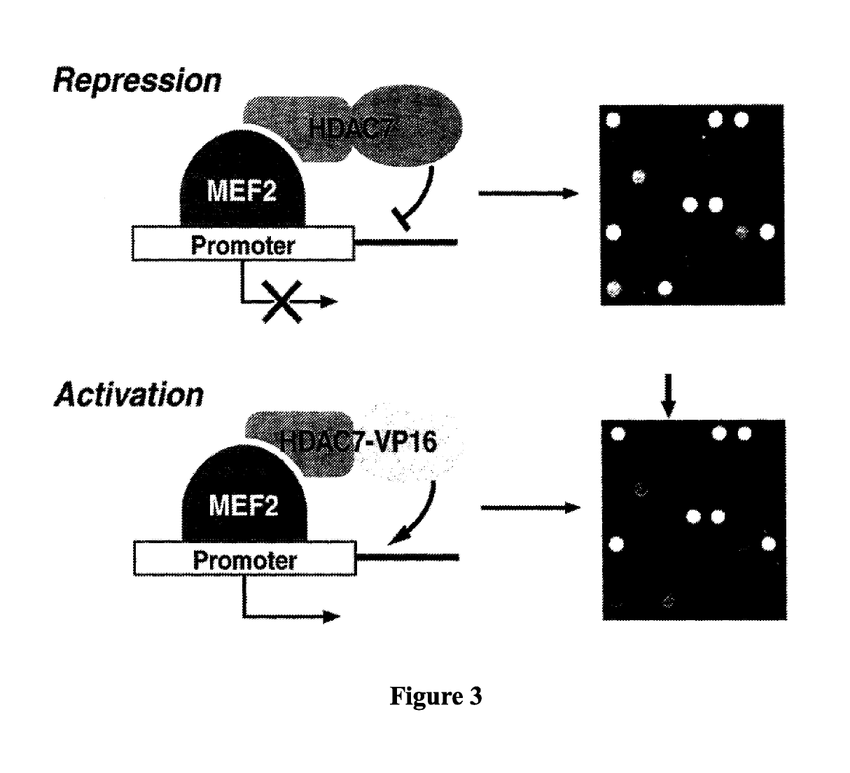

Many signals activate MEF2 and result in cardiac hypertrophy. For example, a variety of stimuli can elevate intracellular calcium, resulting in a cascade of intracellular signaling systems or pathways, including calcineurin, CAM kinases, PKC and MAP kinases and in turn lead to the activation of MEF2 dependent gene activation. It is known that class II HDACs are involved in modulating MEF2 activity (FIGS. 3-5). In order to accomplish this modulation, the class II HDACs must be present in the nucleus of the cell to repress MEF2 driven transcription, and when HDACs are exported out of the nucleus in response to a variety of stimuli (such as phosphorylation), MEF2 genes are activated, leading to hypertrophy and heart failure.

As such, the nuclear compartmentalization of HDAC7 may be a key factor in cardiac disease. HDAC7 which remains in the nucleus or re-enters the nucleus has an anti-hypertrophic function. As such, uncovering a cellular step that keeps HDAC7 in the nucleus, uncovering a way to inhibit nuclear export or uncovering a way to promote or increase re-entry of HDAC7 into the nucleus, represent potential therapeutic targets for the treatment or prevention of hypertrophy, heart failure, or hypertension. Herein, Applicants have shown that dephosphorylation of HDAC7 by myosin phosphatase promotes the re-entry of HDAC7 into the nucleus, where HDAC7 becomes associated with MEF2 and inhibits MEF2 dependent gene activation of MEF2 target genes.

Thus, in accordance with the present invention, a method for the treatment of a pathologic cardiac hypertrophy, heart failure, or hypertension is provided. In a preferred embodiment, this method comprises the steps of (a) identifying a patient having cardiac hypertrophy or heart failure and (b) administering to the patient an effective amount of an activator of myosin phosphatase, wherein the cardiac hypertrophy, heart failure, or hypertension is treated.

In another preferred embodiment, this method comprises the step of (a) identifying a patient having cardiac hypertrophy or heart failure and (b) administering to the patient an effective amount of an inhibitor of HDAC7 nuclear export, wherein the cardiac hypertrophy, heart failure, or hypertension is treated. An inhibitor of HDAC7 nuclear export is a molecule which inhibits or reduces the export of HDAC7 from the nucleus of a cell into the cytoplasm.

The treatment may improve one or more symptoms of cardiac heart failure, such as providing increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, wall tension and wall thickness, quality of life, disease-related morbidity and mortality, decreased remodeling, ventricular dilation, or improving pump performance, decreasing necrosis, arrhythmia, fibrosis, energy starvation or apoptosis. In particular embodiments, the patient is a human.

An activator of myosin phosphatase useful in the subject method is a molecule that increases the dephosphorylation of HADC7, or activates a pathway, mechanism, or protein directly involved in the export of HDAC7 from the nucleus of a cell. This includes proteins, peptides, peptide aptamers, DNA molecules (including antisense), RNA molecules (including RNAi and antisense) and small molecules.

In accordance with the present invention, a method for the prevention of a pathologic cardiac hypertrophy or heart failure is provided. In a preferred embodiment, this method comprises the step of (a) identifying a patient at risk of developing cardiac hypertrophy or heart failure and (b) administering to the patient an effective amount of an activator of myosin phosphatase, wherein the cardiac hypertrophy or heart failure is prevented.

In another preferred embodiment, this method comprises the step of (a) identifying a patient at risk of developing cardiac hypertrophy or heart failure and (b) administering to the patient an effective amount of an inhibitor of HDAC7 nuclear export, wherein the cardiac hypertrophy or heart failure is prevented.

A patient at risk may exhibit one or more of the following: hypertension, uncorrected valvular disease, chronic angina, and/or recent myocardial infarction. Symptoms may include one or more of the following: chest pain, fainting (especially during exercise), light-headedness (especially after activity or exercise), dizziness, sensation of feeling heart beat (palpitations), and shortness of breath. In particular embodiments, the patient is a human.

Hypertension means high blood pressure. This generally means that the systolic blood pressure is consistently over 140 and the diastolic blood pressure is consistently over 90. Pre-hypertension is when the systolic blood pressure is between 120 and 139 and the diastolic blood pressure is between 80 and 89 on multiple readings. Patients with pre-hypertension are likely to develop high blood pressure at some point.

Essential hypertension typically has no identifiable cause. It may be caused by genetics, environmental factors, or diet. Secondary hypertension is high blood pressure caused by a disorder, including, but not limited to, adrenal gland tumors; Xushing's syndrome; kidney disorders (e.g., glomerulonephritis (inflammation of kidneys); renal vascular obstruction or narrowing; renal failure); use of medications, drugs, or other chemicals; oral contraceptives; hemolytic-uremic syndrome; Henoch-Schonlein purpura; periarteritis nodosa; radiation enteritis, retroperitoneal fibrosis, or Wilms' tumor.

3. Asthma

Asthma is a serious chronic condition affecting an estimated 20 million Americans. Asthma is characterized by (i) bronchoconstriction, (ii) excessive mucus production, and (iii) inflammation and swelling of airways. These conditions cause widespread and variable airflow obstruction thereby making it difficult for the asthma sufferer to breathe. Asthma further includes acute episodes or attacks of additional airway narrowing via contraction of hyper-responsive airway smooth muscle.

In asthma, chronic inflammatory processes in the airway play a central role in increasing the resistance to airflow within the lungs. Many cells and cellular elements are involved in the inflammatory process, particularly mast cells, eosinophils T lymphocytes, neutrophils, epithelial cells, and even airway smooth muscle itself. The reactions of these cells result in an associated increase in the existing sensitivity and hyper-responsiveness of the airway smooth muscle cells that line the airways to the particular stimuli involved.

In accordance with the present invention, a method for the treatment of a patient having asthma is provided. In a preferred embodiment, this method comprises the step of (a) identifying a patient having asthma and (b) administering to the patient an effective amount of an activator of myosin phosphatase, wherein asthma is treated.

In another preferred embodiment, this method comprises the step of (a) identifying a patient having asthma and (b) administering to the patient an effective amount of an inhibitor of HDAC7 nuclear export, wherein asthma is treated. An inhibitor of HDAC7 nuclear export is a molecule which inhibits or reduces the export of HDAC7 from the nucleus of a cell into the cytoplasm.

In a further aspect, this invention also provides a method for the prevention of asthma. In a preferred embodiment, this method comprises the steps of (a) identifying a patient at risk of developing asthma and (b) administering to the patient an effective amount of an activator of myosin phosphatase, wherein the asthma is prevented.

In another embodiment of the present invention, the method for the prevention of asthma comprises the steps of (a) identifying a patient at risk of developing asthma and (b) administering to the patient an effective amount of an inhibitor of HDAC7 nuclear export, wherein the asthma is prevented.

A patient having asthma can be identified by e.g., diagnosing (i) bronchoconstriction, (ii) excessive mucus production, and (iii) inflammation and swelling of airways in the patient. Tests may include lung function tests, peak flow measurements, chest x-ray, blood tests (including eosinophil count), or arterial blood gas. Similarly, a patient at risk of developing asthma can be identified using the same tests as above. Symptoms useful for the identification of a patient having asthma or for a patient at risk of developing asthma include wheezing, cough with or without sputum (phlegm) production, shortness of breath which may get worth with exercise or activity, intercostals retractions (pulling of the skin between the ribs when breathing), extreme difficulty breathing, bluish color to the lips and face, severe anxiety due to shortness of breath, rapid pulse, sweating, decreased level of alertness, nasal flaring, chest pain, tightness in the chest, and abnormal breathing pattern.

An inhibitor of HDAC7 nuclear export or an activator of myosin phosphatase identified by one of the subject methods described herein may also be used in a combination therapy with one of the following: (i) inhaled steroids (such as Azmacort® [triamcinolone acetonide], Vanceril® [beclomethasone dipropionate], AeroBid® [flunisolide], Flovent® [fluticasone propionate]), (ii) leukotrine inhibitors (such as Singulair® [montelukast sodium] and Accolate® [zafirlukast]), (iii) anti-IgE therapy (Xolair® [omalizumab]), (iv) long-acting bronchodilators (such as Serevent® [salmeterol xinafoate]), (v) cromolyn sodium (Intal®) or nedocromil sodium, or (vi) aminophylline or theophylline.

### VII. Pharmaceutical Compositions

In one aspect, the present invention provides a pharmaceutical composition or a medicament comprising at least an agent that modulates, the level or activity of a myosin phosphatase and a pharmaceutically acceptable carrier. In a preferred embodiment, the agent reduces the level or activity of the myosin phosphatase. In another preferred embodiment, the agent increases the level or activity of the myosin phosphatase. A pharmaceutical composition or medicament can be administered to a subject for the treatment of or for the prevention of, for example, a pathological condition or disease as described herein.

A. Formulation and Administration

Compounds, agents, and small molecules identified by a method of the present invention, are useful in the manufacture of a pharmaceutical composition or a medicament comprising an effective amount thereof in conjunction or mixture with excipients or carriers suitable for either enteral or parenteral application.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds, agents, and small molecules of the present invention and their physiologically acceptable salts and solvates can be formulated for administration by any suitable route, including via inhalation, topically, nasally, orally, parenterally, or rectally. Thus, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets and capsules can be administered orally, rectally or vaginally.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., a small molecule compound of the present invention, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Compounds, agents, and small molecules of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the compounds, agents, and small molecules may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a compound, agent, and small molecules of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The compounds, agents, and small molecules can also be formulated in rectal compositions, for example, suppositories or retention enemas, for example, containing conventional suppository bases, for example, cocoa butter or other glycerides.

Furthermore, the compounds, agents, and small molecules can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

In one embodiment of the present invention, a pharmaceutical composition or medicament comprises an effective amount of an agent that modulates the level or activity of a myosin phosphatase of the present invention as defined above, and another therapeutic agent. When used with compounds, agents, and small molecules of the invention, such therapeutic agent may be used individually, sequentially, or in combination with one or more other such therapeutic agents (e.g., a first therapeutic agent, a second therapeutic agent, and compounds of the present invention). Administration may be by the same or different route of administration or together in the same pharmaceutical formulation.

B. Therapeutic Effective Amount And Dosing

In one embodiment of the present invention, a pharmaceutical composition or medicament is administered to a subject, preferably a human, at a therapeutically effective dose to prevent, treat, or control a pathological condition or disease as described herein. The pharmaceutical composition or medicament is administered to a subject in an amount sufficient to elicit an effective therapeutic response in the subject. An effective therapeutic response is a response that at least partially arrests or slows the symptoms or complications of the pathological condition or disease. An amount adequate to accomplish this is defined as “therapeutically effective dose.”

The dosage of active compounds administered is dependent on the species of warm-blooded animal (mammal), the body weight, age, individual condition, surface area or volume of the area to be treated and on the form of administration. The size of the dose also will be determined by the existence, nature, and extent of any adverse effects that accompany the administration of a particular small molecule compound in a particular subject. A unit dosage for oral administration to a mammal of about 50 to 70 kg may contain between about 5 and 500 mg of the active ingredient. Typically, a dosage of the active compounds of the present invention, is a dosage that is sufficient to achieve the desired effect. Optimal dosing schedules can be calculated from measurements of compound accumulation in the body of a subject. In general, dosage may be given once or more daily, weekly, or monthly. Persons of ordinary skill in the art can easily determine optimum dosages, dosing methodologies and repetition rates.

In one embodiment of the present invention, a pharmaceutical composition or medicament comprising compounds, agents or small molecules of the present invention is administered in a daily dose in the range from about 1 mg of each compound per kg of subject weight (1 mg/kg) to about 1 g/kg for multiple days. In another embodiment, the daily dose is a dose in the range of about 5 mg/kg to about 500 mg/kg. In yet another embodiment, the daily dose is about 10 mg/kg to about 250 mg/kg. In another embodiment, the daily dose is about 25 mg/kg to about 150 mg/kg. A preferred dose is about 10 mg/kg. The daily dose can be administered once per day or divided into subdoses and administered in multiple doses, e.g., twice, three times, or four times per day. However, as will be appreciated by a skilled artisan, compounds, agents, or small molecules identified by methods of the present invention may be administered in different amounts and at different times.

To achieve the desired therapeutic effect, compounds, agents or small molecules may be administered for multiple days at the therapeutically effective daily dose. Thus, therapeutically effective administration of compounds to treat a pathological condition or disease described herein in a subject requires periodic (e.g., daily) administration that continues for a period ranging from three days to two weeks or longer. Typically, compounds will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the compounds are not administered daily, so long as the administration is repeated frequently enough to maintain a therapeutically effective concentration of the compounds in the subject. For example, one can administer the compounds every other day, every third day, or, if higher dose ranges are employed and tolerated by the subject, once a week.

Optimum dosages, toxicity, and therapeutic efficacy of such compounds, agents and small molecules may vary depending on the relative potency of individual compounds, agents or small molecules and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of compounds is from about 1 ng/kg to 100 mg/kg for a typical subject.

Following successful treatment, it may be desirable to have the subject undergo maintenance therapy to prevent the recurrence of the condition or disease treated.

### VIII. Kits

For use in diagnostic, research, and therapeutic applications suggested above, kits are also provided by the invention. In the diagnostic and research applications such kits may include any or all of the following: assay reagents, buffers, a compound, agent or small molecule of the present invention, a myosin phosphatase polypeptide, a myosin phosphatase nucleic acid, an anti-myosin phosphatase antibody, hybridization probes and/or primers detecting a myosin phosphatase nucleic acid, a myosin phosphatase expression construct, a histone deacetylase polypeptide, a histone deacetylase nucleic acid, a histone deacetylase antibody, hybridization probes and/or primers detecting a histone deacetylase nucleic acid, a histone deacetylase expression construct, a Nur77 polypeptide, a Nur77 nucleic acid, an anti-Nur77 antibody, hybridization probes and/or primers detecting a Nur77 nucleic acid, a Nur77 expression construct, or any other compound or composition described herein. A therapeutic product may include sterile saline or another pharmaceutically acceptable emulsion and suspension base.

Typically, the components of a kit are provided in a container. In a preferred embodiment of the present invention, a kit for reducing, inhibiting or inducing apoptosis comprises a container containing an agent that modulates the level or activity of a myosin phosphatase.

In addition, a kit may include instructional materials containing directions (i.e., protocols) for the practice of the methods of this invention. The instructions may be present in the subject kits in a variety of forms, one or more of which may be present in the kit. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

In a preferred embodiment of the present invention, the kit comprises an instruction for using an agent that increases the level or activity of a myosin phosphatase for reducing or inhibiting apoptosis. In another embodiment, the kit comprises an instruction for using an agent that reduces the level or activity of a myosin phosphatase for inducing apoptosis.

Optionally, the instruction comprises warnings of possible side effects and drug-drug or drug-food interactions.

A wide variety of kits and components can be prepared according to the present invention, depending upon the intended user of the kit and the particular needs of the user.

In a preferred embodiment of the present invention, the kit is a pharmaceutical kit and comprises a pharmaceutical composition comprising (i) an agent that modulates the level or activity of a myosin phosphatase and (ii) a pharmaceutical acceptable carrier. Pharmaceutical kits optionally comprise an instruction stating that the pharmaceutical composition can or should be used for treating a pathological condition or disease described herein.

Additional kit embodiments of the present invention include optional functional components that would allow one of ordinary skill in the art to perform any of the method variations described herein.

Although the forgoing invention has been described in some detail by way of illustration and example for clarity and understanding, it will be readily apparent to one of ordinary skill in the art in light of the teachings of this invention that certain variations, changes, modifications and substitution of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The referenced patents, patent applications, and scientific literature, including accession numbers to GenBank database sequences, referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As can be appreciated from the disclosure above, the present invention has a wide variety of applications. The invention is further illustrated by the following examples, which are only illustrative and are not intended to limit the definition and scope of the invention in any way.

### IX. Examples

### Example 1: General Methods

A. Cell Culture And Cell Treatment

DO11.10 T-cell hybridoma was grown at 37° C. in RPMI 1640 and Dulbecco's modified Eagles medium supplemented with 10% fetal bovine serum, 2 mM glutamine, and 50 U/ml streptomycin/penicillin. DO11.10 cells stably expressing either empty vector, HDAC7-Flag, or HDAC7ΔP-Flag have been described (Dequiedt et al., 2003, Immunity 18:687-698). Mouse primary thymocytes were obtained from 4-6-wk-old Balb/c mice. Where indicated, PMA was added at a concentration of 10 ng/ml, unless indicated otherwise. For CD3 stimulation, tissue culture plates were coated with an anti CD3 antibody (500A2) at a 1:1000 dilution in PBS overnight at 4° C.

B. Cell Transfection Assays

Nucleofection of DO11.10 cells was conducted using Nucleofector Kit R and program 028. Cells were split to 300,000/ml 24 hours before Amaxa nucleofection. Cells (5×10⁶) were spun at 1000 rpm for 10 minutes at room temperature, resuspended in 100 μl of solution R, and nucleofected with 2 μg of either siRNA or expression plasmid by program O28. Nucleofected cells were resuspended in 500 μl of pre-warmed serum-free RPMI lacking antibiotics and allowed to recover at 37° C. in 5% CO₂ incubator for 15 minutes, and 4.5 ml of pre-warmed complete RPMI was added to the cells. Nucleofection of mouse primary thymocytes was performed using the Mouse T cell Nucleofector Kit and program X001, following the manufacturer instructions.

C. Immunoprecipitation

Total cellular extracts from DO11.10 cells or primary thymocytes were prepared in PLB buffer (0.5% Triton-X100, 0.5 mM EDTA, 1 mM DTT in PBS and supplemented with protease inhibitors (Complete, Roche Molecular Biochemicals, Indianapolis, Ind.)). Cellular lysates were precleared with mouse IgG-agarose beads (Sigma, St. Louis, Mo.) for 2 hours at 4° C. Immunoprecipitations of HDAC7-Flag tagged proteins were carried out for 4 hours at 4° C. using anti-M2-agarose beads (Sigma, St. Louis, Mo.) at a concentration of 15 μl/ml. Immunoprecipitated material was washed three times in IPLS buffer (50 mM Tris-HCl, pH7.5, 0.5 mM EDTA. 0.5% NP-40, and 150 mM NaCl) supplemented with protease inhibitors. For immunoprecipitation of endogenous proteins from primary thymocytes, anti-PP 1, anti-PP1β, anti-MYPT1, and anti-14-3-3 antibodies were used at concentrations of 2 g/ml in combination with 50% protein A-Sepharose slurry (Amersham Pharmacia Biotech, Piscataway, N.J.). Immunoprecipitated material was washed three times in IPLS. Bound proteins were subjected to SDS-PAGE and Western blotting.

D. In Vivo HDAC7 Phosphorylation

DO11.10-HDAC7-Flag cells were untreated or treated with either PMA or anti CD3 antibody for the indicated times. Total cellular extracts were prepared in 20 mM Hepes (pH 7.5), 10 mM EGTA, 22.5 mM MgCl₂, 1% NP-40, 2 mM orthovanadate, 1 mM dithiothreitol, and 0.5 mM phenyl-methyl sulfonyl fluoride supplemented with protease inhibitors, and subjected to Western blot analysis.

E. Mass Spectrometry Analysis

DO11.10-Empty and DO11.10-HDAC7-Flag cells were lysed in PLB buffer and the cellular lysates were pre-cleared with mouse IgG-agarose beads for 2 hours at 4° C. HDAC7-FLAG was immunoprecipitated with anti-FLAG M2 agarose affinity gel (Sigma) overnight at 4° C. Immunoprecipitated material was washed three times for 15 min each with lysis buffer and boiled in SDS-sample buffer. The samples were subjected to SDS-PAGE followed by Coomassie blue staining.

Bands corresponding to proteins specifically interacting with HDAC7 were excised (see below) and prepared for mass spectrometry. Gel slices were de-stained in 25 mM ammonium bicarbonate/50% acetonitrile. The gel pieces were treated with 100% acetonitrile until shrinking of the gel pieces was noted. Acetonitrile was removed and the gel pieces were dried in a vacuum centrifuge. Samples were reduced by treatment with 10 mM DTT solution for 45 min at 56° C. The supernatant was removed and the samples were alkylated in 55 mM iodoacetamide solution for 30 min in darkness at room temperature. After washing in 25 mM ammonium bicarbonate for 15 minutes, the gel pieces were treated with 100% acetonitrile for 5 min and completely dried in a vacuum centrifuge. 25 μL of trypsin (12.5 ng/ml) were added to the dried gel pieces followed by incubation on ice for 30 min. 25 mM ammonium bicarbonate was added to cover the gel pieces. After in-gel digestion for 16 h at 37° C., the supernatant was transferred to a fresh tube and peptides were extracted twice by vortexing the gel pieces for 20 min in 50% acetonitrile/5% trifluoroacetic acid. Aqueous and organic peptide extracts were combined and concentrated under vacuum.

Peptide mass fingerprints were obtained by mixing 0.5 μL of each in-gel digest peptide extract with 0.5 μL of matrix solution m-cyano-4-hydroxycinnamic acid, 5 mg/mL in 50% acetonitrile/50% water/0.1% trifluoroacetic acid) directly on a stainless steel target. After co-crystallization of the peptide mixture with the matrix, peptide mass maps were obtained using a Voyager DE STR MALDI-TOF mass spectrometer (Applied Biosystems). In the MALDI-TOF process, peptides were ionized following a matrix-analyte crystal irradiation with a pulsed nitrogen laser (337 nm) that struck the sample at a frequency of 20 Hz. A voltage of 25 kV accelerated the peptide ions out of the ion source into the flight tube after a 125 nanosecond delay. Monoprotonated peptide ions were temporally separated according to their mass-to-charge ratios as they drifted down the flight tube through the reflector mass analyzer eventually striking the detector. Individual peptide masses were determined by measuring the time it took each ion to travel the distance from its origin to the detector. Delayed extraction of peptide ions from the ion source in combination with the kinetic energy focusing properties of the reflector (also called the ion mirror) provided mass resolution sufficient for determining the monoisotopic mass of each peptide. Close proximity external calibration enabled peptide masses to be measured within +50-100 ppm of their theoretical values. Protein identification was accomplished by comparing the experimentally generated set of peptide masses with theoretically predicted sets of tryptic peptides derived from each protein in the Swiss-Prot database, by a process of “in silico digestion.” Database searches were performed using the Aldente Peptide Mass Fingerprinting Tool.

F. SDS-PAGE And Western Blotting

SDS-PAGE and Western blot analysis were performed according to standard procedures. Western blots were developed with the ECL detection kit (Amersham Pharmacia Biotech, Piscataway, N.J.).

G. Plasmid Constructs

The pcDNA3.1-based expression vectors for FLAG-tagged human HDAC7 and FLAG-tagged human HDAC7 phosphorylation mutant (HDAC7Δ) have been described in Dequeidt et al. (2003, Immunity 18(5):687-98).

H. Antibodies

Anti-FLAG (α-FLAG) antibodies were obtained from Sigma. Anti-PKD1 (α-PKD1) antibodies, anti-PP1β (α-PP1β) antibodies, anti-PP1γ (α-PP1γ) antibodies, anti-PP2B (α-PP2B) antibodies, anti-14-3-3ε (α-14-3-3ε) antibodies, anti-14-3-3β (α-14-3-3β) antibodies, anti-14-3-3θ (α-14-3-3θ) antibodies, and anti-actin (α-Actin) antibodies were obtained from Santa Cruz Biotechnology. Anti-actin (α-Actin) antibodies were also obtained from Sigma. Anti-MYTP1 (α-MYTP 1) antibodies were obtained from Abcam. Anti-PP1α (α-PP1α) antibodies, anti-PP1β (α-PP1β) antibodies, and anti-PP2A (α-PP2A) antibodies were obtained from Upstate Biotechnology. Anti-CD3 (α-CD3) antibodies and anti-Nur77(α-Nur77) antibodies were obtained from BD Pharmingen.

I. Immunofluorescence

DO11.10 cells (5×10⁶) were nucleofected with an HDAC7-GFP expression vector together with either siRNA control or siRNAs for PP1β and MYPT1. Cells (5×105) were seeded onto poly-L-lysine-coated coverslips 12 h after nucleofection and allowed to attach for 12 h. Cells were stimulated with 10 ng/ml PMA. HDAC7 was localized by immunofluorescence microscopy with a confocal fluorescence microscope (Olympus BX60, Bio-Rad).

J. Si RNA Inhibition (RNA Interference)

SiRNA inhibition was performed as follows. Pre-designed siRNA pools targeting transcripts of the mouse PP1α, PP1β, PP1γ, and MYPT1 genes as well as control siRNA pool were used to knockdown the respective genes in DO11.10 cells and mouse primary thymocytes. siControl and siMYPT1 were from Dharmacon. The siRNAs for the different PP1 isoforms were from Ambion. siRNAs were delivered by Amaxa nucleofection.

The following oligonucleotide(s) were used for inhibiting PP1α expression from PP1α mRNA (siPP1α): 5′-GAACGUGCAGCUGACAGAGtt-3′,5′-GGGCAAGUAUGGGCAGUUCtt-3′, and 5′-GGUUGUAGAAGAUGGCUAUtt-3′.

The following oligonucleotide(s) were used for inhibiting PP1β expression from PP1β mRNA (siPP1β): 5′-CCAGAAGCCAACUAUCUUUtt-3′,5′-GCCAACUAUCUUUUCUUAGtt-3′, and 5′-CGGAUAUGAAUUUUUUGCUtt-3′.

The following oligonucleotide(s) were used for inhibiting PP1γ expression from PP1γ mRNA (siPP1γ): 5′-CCGAUAAUGCUUUCUUUGGtt-3′,5′-GCAAGCCAAGCACUUCAUUtt-3′, and 5′-CGGGCAGUACUAUGAUUUGtt-3′.

The following oligonucleotide(s) were used for inhibiting MYPT1 expression from MYPT1 mRNA (siMYPT1): 5′-GAACGAGACUUGCGUAUGUUU-3′,5′-AAGAAUAGUUCGAUCAAUGUU-3′,5′-CGACAUCAAUUACGCCAAUUU-3′ and 5′-UCGGCAAGGUGUUGAUAUAUU-3′.

The following non-targeting oligonucleotide(s) were used as control oligonucleotides in RNAi experiments (siCo): 5′-AUGAACGUGAAUUGCUCAA-3′,5′-UAAGGCUAUGAAGAGAUAC-3′,5′-AUGUAUUGGCCUGUAUUAG-3′ and 5′-UAGCGACUAAACACAUCAA-3′. Some experiments were also performed using an siRNA control targeting the GL3 luciferase mRNA (Dharmacon) and having the sequence 5′-CUUACGCUGAGUACUUCGAtt-3′.

Oligonucleotide(s) for inhibiting MYTP 1 expression from MYTP 1 mRNA (siMYTPT1) and control oligonucleotides (siCo) were obtained from Dharmacon (“smart pool”).

K. Nucleofection

DO11.10 cells were transfected using the Amxa nucleofector kit R and program O28. Cells were split to 3×10⁵ cells/ml 24 h before nucleofection. Cells (5×10⁶) were spun at 1,000 rpm for 10 min at room temperature, resuspended in 100 μl of solution R, and nucleofected with 2 μg of either siRNA or expression plasmid by program 028. Nucleofected cells were resuspended in 500 μl of prewarmed serum-free RPMI lacking antibiotics and allowed to recover for 15 min at 37° C. in a 5% CO₂ incubator, and 4.5 ml of prewarmed complete RPMI was added to the cells. Nucleofection of mouse primary thymocytes was performed with the Mouse T-cell Nucleofactor Kit and program X001, following the manufacturer's instructions.

L. Flow Cytometry

Postnatal human thymus specimens were obtained from patients undergoing cardiac surgery (Moffitt Hospital at University of California, San Francisco) and were processed within 6 hr. After mechanical disruption of thymus fragments, single-cell suspensions of thymocytes were stained with a mAb cocktail containing CD4-PE (Becton Dickinson), CD8-Tricolor (Becton Dickinson), and CD3-FITC (Becton Dickinson). A FACS Vantage (Becton Dickinson) was used to purify five thymocyte subsets: CD3+CD4+CD8⁻ (SP4), CD3⁺ CD4⁻ CD8⁺ (SP8), CD31^{low}CD4⁺CD8⁺ (DP^{low}), CD3^{med/high}CD4⁺CD8⁺ (DP^{medium/high}), and CD3⁻CD4⁻ CD8⁻ (TN). Typically, the purity of sorted cells was greater than 97%.

M. RT-PCR

Total RNA was extracted, e.g., from frozen pellets (−10⁵ cells) with Trizol (Gibco BRL) or from cultured cells according to the manufacturer's instructions. RNA was treated with DNaseI (RQ1 RNase-Free DNase, Promega) to ensure total removal of genomic DNA. First-strand cDNA (20 μl) was generated from isolated RNA with the SuperScript First-Strand Synthesis System for RT-PCR (Gibco BRL) as described by the manufacturer. HDAC mRNAs were quantified with the TaqMan fluorogenic detection system on an ABI Prism 7700 Sequence Detector (Perkin-Elmer Applied Biosystems). PCR reactions were performed in duplicate on two dilutions of first strand cDNA with the following primers: HDAC4 forward 5′-TGACCGCCATTTGCGA-3′, HDAC4 reverse 5′-CGTTTCCCAGCAAGGCA-3′, HDAC5 forward 5′-TGGTCTACGACACGTT CATGCT-3′, HDAC5 reverse 5′-TCAGGGTGCACGTGTGTGTT-3′, HDAC7 forward 5′-TGGTGTCTGCTGGATTTGATG-3′, HDAC7 reverse 5′-ATCCAAAACATTTGGCAGAAACAT-3′. MGB-5′-CCTCGGAAGCATGTGTTA-3′, MGB-5′-CACCAGTGCATGTGC-3′, and FAM-5′-CCGGCCCCACTGGGTGGCTA-3TAMRA (Operon, CA) were used as HDAC4, HDAC5, and HDAC7-specific probe, respectively. PCR amplification consisted of denaturation at 95° C. for 10 min, followed by 40 cycles of denaturation at 95° C. for 15 s and annealing/extension at 58° C. for HDAC7 or 60° C. for HDAC4/5 for 60 s. For GAPDH detection, the TaqMan GAPDH control reagents kit (Applied Biosystems, CA) was used with an annealing/extension step at 60° C. Standard curves were plotted for HDACs and GAPDH. For each sample, HDAC expression was normalized to GAPDH.

N. Northern Blot Analysis

The tissue expression of HDAC7 was analyzed with a multiple human tissue Northern blot and RNA master blots from Clontech. Total RNA (5 g) isolated with Trizol was used to detect the Nur77 message by standard Northern blot analysis (Sambrook et al., 1989, Molecular Cloning: A Laboratory Manual, Second Edition (Cold Spring Harbor, N.Y.; Cold Spring Harbor Laboratory Press)). ³²P-labeled probes corresponding to human HDAC7, mouse Nur77, or human GAPDH were prepared with the Megaprime DNA labeling system (Amersham Pharmacia Biotech). Blots were prehybridized and hybridized with ExpressHyb hybridization solution (Clontech) and washed under high stringency conditions (Sambrook et al., 1989, Molecular Cloning: A Laboratory Manual, Second Edition (Cold Spring Harbor, N.Y.; Cold Spring Harbor Laboratory Press)). Autoradiographs were analyzed with a FUJIX BAS 1000 phosphor imaging system (Fuji, Tokyo, Japan).

O. In Situ Hybridization

In situ hybridization was performed according to Mannheim (1996, Nonradioactive In Situ Hybridization Application Manual, Second Edition, Roche Diagnostic Corporation)). Sense and antisense digoxigenin-labeled human HDAC7 riboprobes and other riboprobes were prepared with the Dig RNA Labeling Kit (Boehringer Mannheim) and shortened to 150-300 base fragments by alkaline hydrolysis. Sections of formalin-fixed paraffin-embedded tissue (4 μm thick) were deparaffinized, hydrated, pretreated with 0.2N HCl for 10 min and digested with proteinase K (Dako) for 25 min. The tissue was covered with probe solution (0.5 ng/μl) and hybridized overnight at 55° C. Excess probe was removed by stringent, 2×SSC for 15 min at 42° C., and 0.1×SSC for 15 min at 42° C. The sections were incubated with anti-digoxigenin Fab fragments conjugated with alkaline phosphatase diluted 1:300 (Boehringer Mannheim) for 30 min, followed by the substrate BCIP/NBT (Vector Laboratories, Burlingame, Calif.) and developed overnight. The slides were washed and then counterstained with Nuclear Fast Red (Vector Laboratories).

P. Protein Kinase Assays

Immunoprecipitated PKD 1 was incubated with myelin basic protein or purified GST-HDAC7 fusion proteins. Phosphorylation reactions were performed in 301 of PKD1 kinase buffer supplemented with 20 μM ATP and 5 μCi of [γ-³²P]ATP at 30° C. for 30 min. Reactions were stopped by the addition of 4× Laemmli sample buffer and resolved by SDS-PAGE on 8% gels.

Q. In Vitro Dephosphorylation Assays

Lysates were prepared from DO11.10-HDAC7-Flag cells either untreated or treated with PMA for 30 min. Cell lysates were immunoprecipitated and washed as described herein. Washed beads were resuspended in 20 μl of phosphatase assay buffer (50 mM Tris-HCl at pH 7.5, 0.1% 2-(3-mercaptoethanol, 0.1 mM EDTA, 1 mg·ml BSA) and treated with 10 U of CIP (New England Biolabs), or 0.5 U of a mixture of recombinant PP1 isoforms (Upstate Biotechnology) was added to the beads. The dephosphorylation reaction was carried out for 30 min at 30° C. The samples were subjected to Western blot analysis.

R. Apoptosis Analysis

Primary thymocytes were nucleofected with 2 ug of the indicated siRNA and expression plasmid. 16 hours after nucleofection, 10⁶ cells were plate in triplicate onto the anti-CD3 coated wells. After 24 hours, thymocytes were stained with AnnexinV-APC, anti-CD4-PE and anti-CD8-FITC (all of them from BD Pharmingen), and analyzed on a FACSCalibur (Becton Dickinson) with CellQuest software. Apoptosis represents the percentage of double-positive thymocytes positive for Annexin V staining. Viability represents the % of double positive (DP) thymocytes negative for AnnexinV staining.

S. Statistical Analysis

Statistical analysis was performed with SPSS 10.0 (SPSS). Differences between means were assessed by ANOVA, followed by Tukey-Kramer post hoc test.

### Example 2: Expression and Function of HDAC7

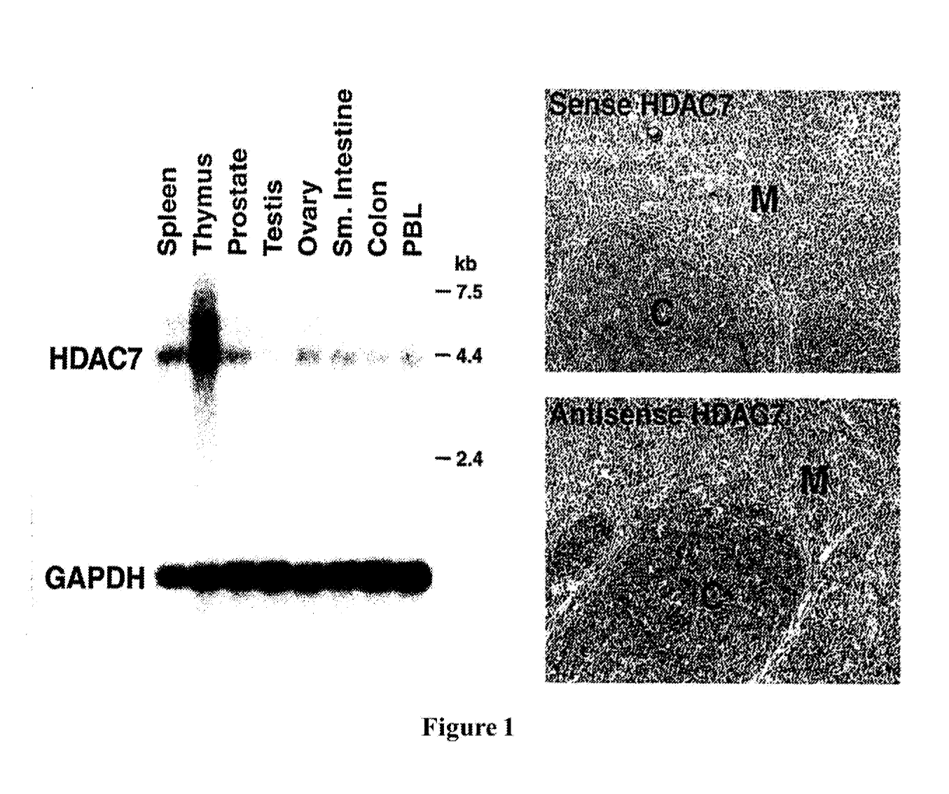

Northern analysis revealed that HDAC7 is highly expressed in the thymus (FIG. 1). In situ hybridization further revealed that HDAC7 was expressed in cortical lymphocytes within the thymus (FIG. 1).

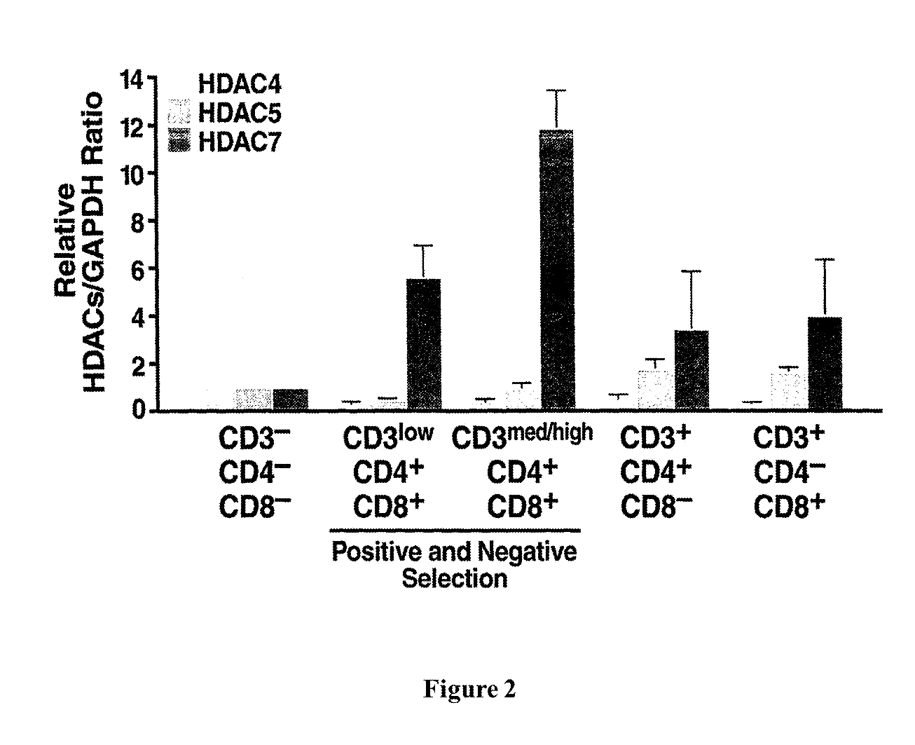

Separation of thymic lymphocytes based on CD3, CD4 and CD8 by FACS showed that HDAC7 is present at highest levels in the double positive, CD4 and CD8 thymocytes, and that its expression significantly decreases in single positives, CD4 and CD8 thymocytes (FIG. 2). These observations suggested a possible role of HDAC7 in the process of positive or negative selection. In resting double positive thymocytes, HDAC7 is a predominantly nuclear protein where it represses its target genes.

Applicants observed that activation of thymocytes via their T cell receptor rapidly leads to the phosphorylation of three residues in the N-terminal domain of HDAC7, to the recognition of these phosphorylated residues by 14-3-3 adaptor proteins and to the nuclear-cytoplasmic transport of HDAC7. The removal of HDAC7, a transcriptional repressor, from its target genes leads to their transcriptional activation.

### Example 3: Identification Of Genes Regulated By HDAC7

To identify the genes that are regulated by HDAC7, two new mutated versions of HDAC7 were generated. The first construct transformed HDAC7 from a repressor to an activator. In this construct, the catalytic deacetylase domain of HDAC7, which functions as a repressor, was substituted by the VP16 transactivating domain. This substitution should transform HDAC7 from a repressor to a transcriptional activator. By profiling gene expression in cells expressing this HDAC7-VP 16 construct in comparison to cells expressing wild type HDAC7 protein, primary and secondary targets of HDAC7 were identified. A typical example of a microarray is shown in FIG. 3 with a single gene lighting up in response to HDAC7-VP 16.

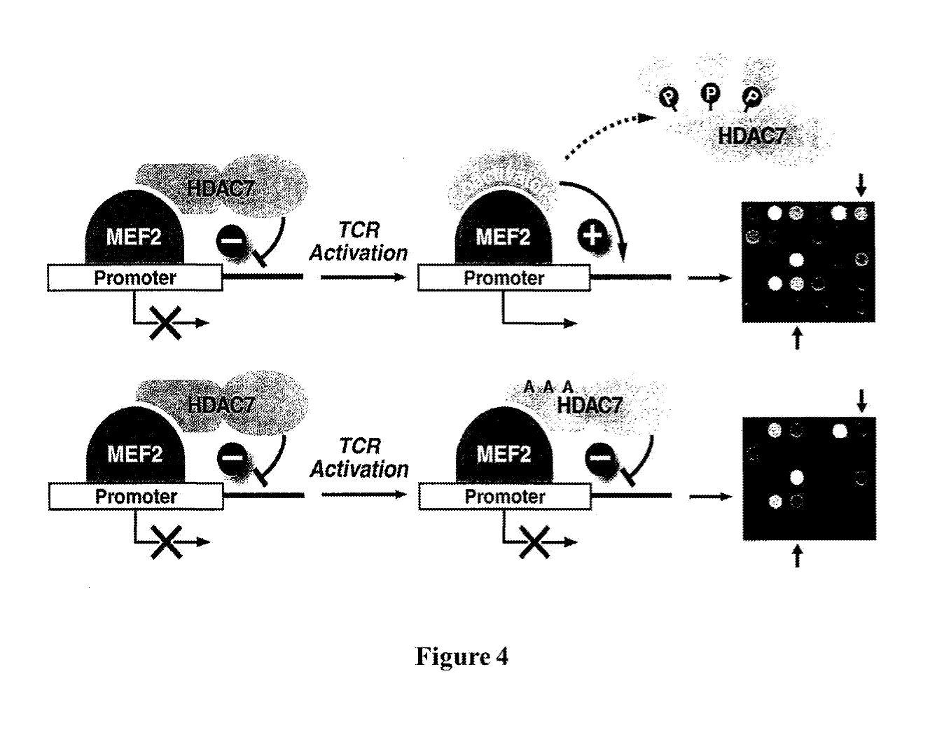

The second construct attempted to block the nucleocytoplasmic shuttling of HDAC7. As shown by Applicants, HDAC7 becomes phosphorylated after TCR activation, leading to its nucleocytoplasmic shuttling. TCR activation also lead to the recruitment of transcriptional coactivators, which bind to MEF2 and lead to the transcriptional activation of the genes that were repressed by HDAC7. Applicants identified the sites of phosphorylation of HDAC7 and found that mutation of these sites (serine residues as described herein) locked the nucleocytoplasmic shuttling of HDAC7 in response to TCR activation. By overexpressing this mutated HDAC7 construct in cells and profiling their gene expression after TCR activation in comparison to cells expressing wild type HDAC7, the activation of a subset of genes in response to TCR activation should be blocked (FIG. 4).

A subset of the genes that were identified using this approach and which were subsequently validated using Northern blot analysis is shown in FIG. 5. These genes fall in three groups, responding to HDAC7-VP 16 alone, suppressed by HDAC7-delta P (the HDAC7 mutant that cannot be phosphorylated during TCR activation), or modulated by both constructs. Many of these genes are previously identified molecules that participate in thymocyte signaling, apoptosis and differentiation. Quite interestingly, many of the genes identified are also transcriptionally activated during positive selection, (e.g., HDAC5, CD28 antigen, CD5 antigen, CD6 antigen, Cytohesin-binding protein, Tripartite motif-containing 35, Sialytransferase 8, Sialyltransferase 9, Nur77, Programmed cell death 1, Chemokine (C-C motif) receptor 8. TDAG8, G-protein-coupled receptor 146, Integrin β2 (LFA-1, CD18), ζ-chain (TCR)-associated protein kinase, Dual-specificity phosphatsee 10 (MKP5), Dual-specificity phosphatase 2 (PAC-1), Diacylglycerol kinase ζ, Friend Leukemia Integration 1, Ankyrin repeat and SOCS box-containing protein 6, Ngfi-A binding protein; Lymphotoxin B, Tumor necrosis factor, Scotin gene, Rlk-Tk-binding protein, and Lck-associated adaptor protein), while a smaller subset is activated during negative selection (e.g., CD6 antigen, OX40 antigen, GADD 45 β, Cytohesin-binding protein, Nur77, Interferon regulatory factor 8, Interferon regulatory factor 4, CD137 (4-1BB), Tribbles homolog 1, Reticuloendotheliosis oncogene, Ngfi-A binding protein; Tumor necrosis factor receptor superfamily member 19, Lymphotoxin A, Lymphotoxin B, Tumor necrosis factor, Scotin gene, Cytokine-inducible SH2-containing protein; pP21 (waf1), and Lunatic fringe gene homolog (Drosophila). A significant fraction of these genes are activated both during negative and positive selection.

### Example 4: HDAC7-Specific Phospho Antibodies Demonstrate HDAC7 Phosphorylation after Stimulation with PMA or TCR Activation and Rapid Dephosphorylation

Parra et al. and Dequiedt et al. reported the identification of a kinase that links TCR activation with HDAC7 (Parra et al., J Biol Chem Biol 280(14):13762-13770; Dequiedt et al., 2003, Immunity 18:687-698). It is called protein kinase D, or PKD, and has also been implicated in lymphocyte signaling by Doreen Cantrell and her group. Recently, it was reported that PKD also phosphorylated HDAC5, another class IIa HDAC that is expressed in heart (Matthews et al., 2006, Mol Cell Biol 26(4):1569-77). To further study this process, three antisera specific for each of the phosphorylation sites of HDAC7 were generated.

Antibodies against mouse HDAC7 phosphorylated at serine residues 178, 344 and 479 were generated (Sigma Genosys, INC., Houston Tex. 77216-1508, USA). Briefly, rabbits were immunized with KLH-conjugated peptides. The phosphopeptides used to generate the phosphor-specific antibodies were FPLRTV[pSer]EPNLKL for P-Ser178, RPLNRTR[pSer]EPLPPS for P-Ser344 and RPLSRTQ[pSer]SPAAPV for P-Ser479. [pSer] indicates the phosphate group on the conserved serine residues. HDAC7 phospho specific antibodies were purified from crude rabbit serum by double affinity purification with nonphosphorylated and phosphorylated peptide. The corresponding serine residues can be found in human HDAC7 at amino acid positions 155, 318, and 448, respectively.

The specificity of these antisera is demonstrated by the fact that they only recognize immunoprecipitated HDAC7 from cells treated with PMA, or CD3 crosslinking and by the observation that dephosphorylation of HDAC7 in vitro with calf intestinal phosphatase abrogates the signal detected with each of these antisera.

Using these antisera, the state of phosphorylation of HDAC7 following PMA treatment was assessed. Western blotting of immunoprecipitated HDAC7 from a thymocyte hybridoma cell line (DO11.10) stably expressing HDAC7-Flag showed increased HDAC7 phosphorylation at each serine after treatment with PMA (FIGS. 6A, 6B) or TCA activation via CD3 cross-linking (FIG. 6C). All three residues showed some degree of phosphorylation under basal conditions, consistent with the observation that HDAC7 occurs both in the cytoplasm and nucleus of untreated DO11.10 cells (FIG. 6A; Parra et al., J Biol Chem 280:13762-13770). Phosphatase treatment of immunoprecipitated HDAC7 abolished the reactivity of the different phosphor-HDAC7 antibodies confirming their specificity for phosphorylated HDAC7 (FIG. 6A).

Time-course analysis of HDAC7 phosphorylation after PMA treatment showed that HDAC7 was rapidly phosphorylated at the three conserved serine residues, reaching a maximum after 1-2 h of treatment with PMA (FIG. 6B). Unexpectedly, a progressive decrease in phosphorylation was observed for all three residues starting at 4 h after PMA treatment (FIG. 6B), while the total HDAC7 content did not change (FIG. 6B, α-Flag Western blot). Similar results were observed in response to CD3 crosslinking (FIG. 6C).

To test the possibility that HDAC7 became dephosphorylated by a phosphatase, cells were stimulated with PMA followed by the addition of okadaic acid, a phosphatase inhibitor, for the rest of the time course. Consistent with the hypothesis that HDAC7 is dephosphorylated by a phosphatase, okadaic acid treatment led to persistent phosphorylation of HDAC7 at each site up to 24 h (FIG. 6B). These data are consistent with the existence of a phosphatase responsible for the rapid dephosphorylation of serine 155, serine 318, and serine 448 after stimulation.

### Example 5: Subcellullar Localization Of HDAC7 Paralleles Phosphorylation Of HDAC7

To determine whether the changes in HDAC7 phosphorylation affected the subcellular localization of the protein, the fate of an HDAC7-GFP fusion protein in response to PMA was followed. As reported, HDAC7-GFP was present both in the nucleus and cytoplasm under basal conditions and was rapidly excluded from the nucleus after PMA stimulation (FIG. 7A; Dequiedt et al., 2003, Immunity 18:687-698); Parra et al., 2005 J Biol Chem 280:13762-13770). The subcellular localization of HDAC7 also closely paralleled serine 155, serine 318, and serine 448 phosphorylation. While resting cells did show nuclear exclusion of HDAC7, more than 75% of activated cells excluded HDAC7 from the nucleus at 2 hours after stimulation (FIG. 7A). However, this exclusion was transient and HDAC7 progressively returned to the nucleus over the next few hours (FIGS. 7A, 7B). Significant reimport of HDAC7-GFP into the nucleus occurred at 4 h after PMA treatment, a time course that paralleled HDAC7 dephosphorylation (FIGS. 7A, 7B). By 24 hours, <20% of the cells showed nuclear exclusion of HDAC7 (FIGS. 7A, 7B). Based on these data, it was hypothesized that a phosphatase was responsible for the dephosphorylation of HDAC7 and its return to the nucleus. Phosphatases have been predicted to regulate the nucleo-cytoplasmic shuttling of other class IIa HDAC, such as HDAC4, 5, and 9 for some time but have not been identified so far.

### Example 6: Activation of the Nur77 Gene after PMA Treatment is Transient

Applicants have previously shown that HDAC7 is the main Class IIa HDAC expressed in the thymus (Dequiedt et al., 2003, Immunity, 18:687-698; incorporated herewith by reference in its entirety). Under basal conditions, HDAC7 is mainly present in the nucleus of T cells repressing the Nur77 gene. Nur77 plays a key role in the induction of negative selection or apoptosis of T cells. The HDAC7-mediated Nur77 repression results in the inhibition of apoptosis. Applicants recently showed that, after TCR activation, the serine/threonine kinase PKD1 phosphorylates HDAC7 at three conserved serine residues leading to its nuclear export and to the transcriptional activation of Nur77 (Parra et al., 2005, J Biol Chem 280(14):13762-13770; incorporated herewith by reference in its entirety).

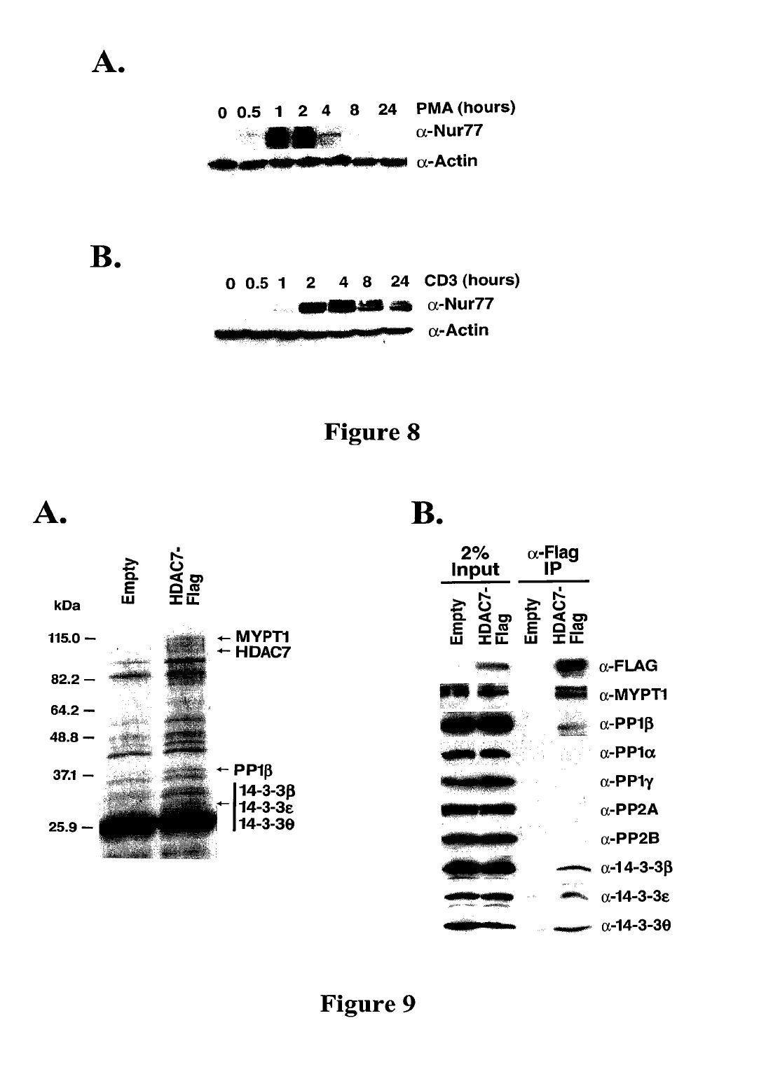

In agreement with the observation of HDAC7 serine 155 phosphorylation, HDAC7 nucleocytoplasmic shuttling correlated with a rapid and transient induction of its target gene, Nur77. Induction of Nur77 peaked at 2 h after PMA treatment (FIG. 8A) and 2-4 hours following CD3 crosslinking (FIG. 8B) and disappeared rapidly thereafter. The kinetics of HDAC7 phosphorylation and dephosphorylation were slightly delayed when the cells were stimulated through CD3 cross-linking: HDAC7 became fully phosphorylated at 4 h after stimulation and was completely dephosphorylated after 8-14 h (FIG. 6C).

### Example 7: Identification of the HDAC7 Phosphatase, Myosin Phosphatase

An open question that remains to be addressed in the field of Class IIa HDACs is the identification of a phosphatase dephosphorylates them in the cytoplasm resulting in their nuclear relocalization and in the repression of their target genes.

To identify a potential HDAC7 phosphatase, a cell line stably expressing a FLAG-tagged HDAC7 in the T cell hybridoma DO11.10 (DO11.10-HDAC7-Flag cells) was constructed. As negative control a DO11.10 cell line expressing empty vector (DO11.10-Empty) was used. HDAC7-Flag tagged protein was immunoprecipitated using an anti-FLAG antiserum. The purified HDAC7 complex was then subjected to SDS-PAGE and Coomassie staining (FIG. 9) followed by mass spectrometry analysis of the differential bands that were pulled down with HDAC7. The mass spectrometry analysis of the purified peptides was performed at BRC Mass Spectrometry facility, University of California San Francisco. As reported for class IIa HDACs (Grozinger and Schreiber 2000, Proc Natl Acad Sci USA 97:7835-7840; Wang et al., 2000, Mol Cell Biol 20:6904-6912), different 14-3-3 isoforms β, ε, and θ also coimmunoprecipitated with HDAC7 (FIG. 9A) However, surprisingly, it was found that the protein phosphatase PP1 isoform, PP1β, and the myosin phosphatase target subunit, MYPT1, were also present in the HDAC7 complex in DO.11.10 cells and found to be associated with HDAC7. MYPT1 is a specific PP1β regulatory subunit. Specifically, MYPT1 is an adaptor protein that mediates the binding of the catalytic subunit, PP1β, to the phosphorylated substrate (Ito et al., 2004, Nature 367:281-284). Both proteins, MYPT1 and PP1β, are part of a complex called myosin phosphatase, which contains a third subunit called M20.

The following peptide sequences were identified as corresponding to mouse 14-3-3 protein epsilon (P62259, web site for National Center for Biotechnology Information (NCBI)) and human 14-3-3 protein epsilon (P62258, web site for NCBI): NH₂-YLAEFATGNDRK-COOH, NH₂-NLLSVAYKNVIGAR-COOH, NH₂-MDDREDLVYQAK-COOH, NH₂-AASDIAMTELPPTHPIR-COOH, and NH₂-LAEQAERYDEMVESMK-COOH.

The following peptide sequences were identified as corresponding to mouse 14-3-3 protein eta (P68510, web site for NCBI): NH₂-GDREQLLQR-COOH, NH₂-LAEQAERYDDMASAMK-COOH, NH₂-EAFEISKEHMQPTHPIR-COOH, NH₂-NSVVEASEAAYKEAFEISK-COOH, and NH₂-AVTELNEPLSNEDRNLLSVAYK-COOH.

The following peptide sequences were identified as corresponding to mouse 14-3-3 protein zeta/delta (Protein kinase C inhibitor protein 1) (KCIP-1) (SEZ-2) (P63101, web site for NCBI): NH₂-LAEQAER-COOH, NH₂-SVTEQGAELSNEER-COOH, NH₂-NLLSVAYK-COOH, NH₂-VVSSIEQK-COOH, NH₂-VVSSIEQKTEGAEKK-COOH, NH₂-FLIPNASQPESK-COOH, NH₂-YLAEVAAGDDKK-COOH, NH₂-EMQPTHPIR-COOH, and NH₂-ACSLAK-COOH.

The following peptide sequences were identified as corresponding to mouse 14-3-3 protein gamma (P61982, web site for NCBI): NH₂-LAEQAER-COOH, NH₂-LAEQAERYDDMAAAMK-COOH, NH₂-NLLSVAYK-COOH, NH₂-VISSIEQK-COOH, NH₂-KIEMVR-COOH, NH₂-IEMVR-COOH, NH₂-YLAEVATGEK-COOH, NH₂-YLAEVATGEKR-COOH, NH₂-ATVVESSEK-COOH, and NH₂-AYSEAHEISK-COOH.

The only peptide sequence identified differentiating 14-3-3 protein theta (P68254, web site for NCBI) from other 14-3-3 species detected in the same sample was NH₂-NVVGGRR-COOH.

The only peptide sequence identified differentiating 14-3-3 protein beta/alpha (Q9CQV8, web site for NCBI) from 14-3-3 protein gamma detected in the same sample was NH₂-GDYFR-COOH.

The following peptide sequences were identified as corresponding to mouse PP1β protein (Serine/threonine-protein phosphatase PP1-beta catalytic subunit (EC 3.1.3.16) (P62141, web site for NCBI): NH₂-IYGFYDECKR-COOH, NH₂-IYGFYDECKRR-COOH, NH₂-YQYGGLNSGRPVTPPR-COOH, and NH₂-TANPPKKR-COOH.

The following peptide sequences were identified as corresponding to mouse MYPT1 protein (Protein phosphatase 1 regulatory subunit 12A (Myosin phosphatase targeting subunit 1) (Q9 DBR7, web site for NCBI): NH₂-LAYVTPTIPR-COOH, NH₂-TSSSYTR-COOH, NH₂-SCSFGR-COOH, and NH₂-SLPSSTSTAAK-COOH.

Each of the identified protein identifiers (e.g., Q9 DBR7) at the NCBI web site allows the identification of the respective nucleotide sequence encoding such protein.

Myosin phosphatase dephosphorylates myosin light chain (MLC), leading to the relaxation of smooth muscle cell (Ceulemans and Bollen 2004, Physiol Rev 84:1-39; Ito et al., 2004, Nature 367:281-284). Myosin phosphatase has been extensively studied in smooth muscle cells where it controls the levels of phosphorylation of myosin and counteracts the activity of myosin kinase. Both proteins control muscle tone in smooth muscle cells, myosin kinase positively and myosin phosphatase negatively. In this cell type, myosin phosphatase is under the control of the Rho protein and Rho kinase which negatively regulate its activity.

To confirm that myosin phosphatase interacts with HDAC7, HDAC7 was immunoprecipitated from DO11.10-HDAC7-Flag cells and probed for its association with endogenous PP1β and MYPT1. The immunoprecipitations showed that HDAC7 associated with both proteins, i.e., MYPT1 and PP1β (FIG. 9B). To further analyze the specificity of this interaction, the potential interaction with other PP1 isoforms, such as PP1α and PP1γ, as well as the serine/threonine phosphatases PP2A and Calcineurin/PP2B was tested using specific antibodies directed to these phosphatases. Interestingly, none of them were found to interact with HDAC7 (FIG. 9B). Further, HDAC7 kinase, PKD1, and different 14-3-3 isoforms also were found to interact with HDAC7 (FIG. 9B and data not shown).

### Example 8: Myosin Phosphatase Interacts with HDAC7 in Mouse Primary Thymocytes

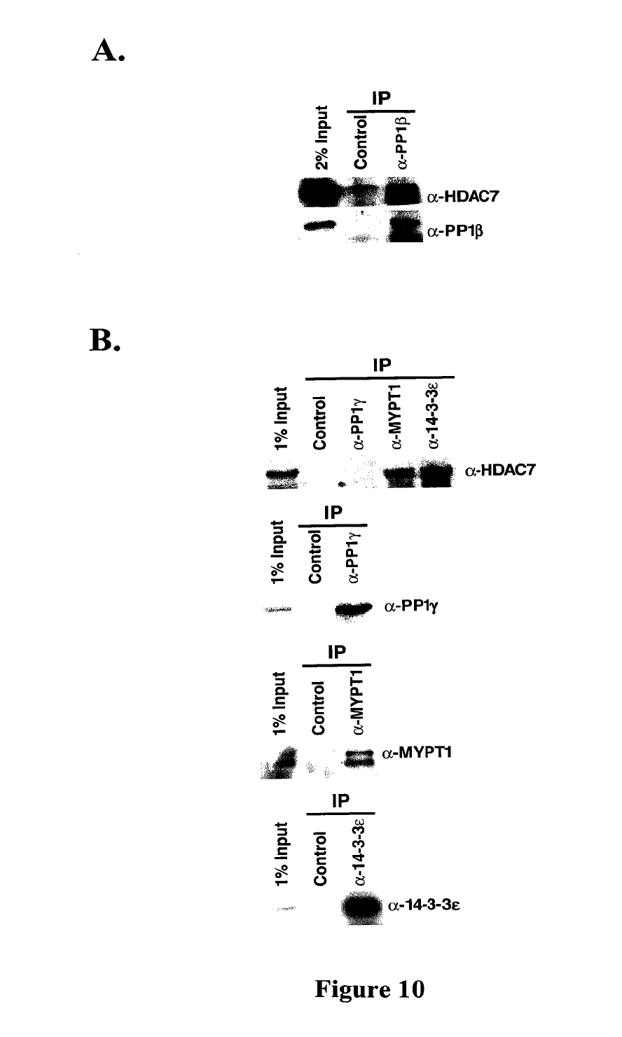

To demonstrate that HDAC7 also interacts with myosin phosphatase, i.e., MYPT1 and PP1β, in other cells, coimmunoprecipitation experiments similar to those described above, were performed in mouse primary thymocytes. The Western blot analyses of these immunoprecipitated proteins showed that endogenous HDAC7 also coimmunoprecipitated with MYPT1 and PP1β in mouse primary thymocytes (FIGS. 10 A, 10B). 14-3-3 was also found to interact with HDAC7, whereas PP1γ was not (FIG. 10B). Taken together, these results demonstrate that myosin phosphatase specifically interacts with HDAC7 in the DO11.10 T cell hybridoma and in mouse primary thymocytes.

### Example 9: Myosin Phosphatase Dephosphorylates and Regulates HDAC7

To study the phosphorylation/dephosphorylation of HDAC7 in vivo, specific phospho antibodies for each of the three conserved serines on HDAC7 were generated (see Example 4). The phospho-HDAC7 antibodies were tested by Western blot analysis of immunoprecipitated HDAC7-Flag tagged protein from DO11.10 cells untreated or treated with PMA for 30 minutes. An increase in HDAC7 phosphorylation at each of the serines was observed after PMA treatment (FIG. 11A).

To test whether PP1 dephosphorylates HDAC7, the immunoprecipitated HDAC7-Flag from cells treated or not with PMA were incubated with a mixture of recombinant PP1 isoforms (α, β, and γ; Upstate) and subject to a dephosphorylation assay for 30 minutes at 30° C., followed by Western blot analysis. It was found that recombinant PP1 treatment totally abolished the reactivity to the different phospho HDAC7 antibodies demonstrating that proteins of the PP1 family dephosphorylate HDAC7 in vitro (FIG. 11A).

To further probe the role of myosin phosphatase in the dephosphorylation of HDAC7 in vivo, small interfering RNAs (siRNAs) specific for PP1β and MYPT1 were introduced into DO11.10 cells expressing HDAC7. With an siRNA control (siCo), HDAC7 was transiently phosphorylated in response to PMA, with a peak at 2 h, and rapidly dephosphorylated thereafter (FIG. 11B, left panel). In contrast, when siRNAs specific for the myosin phosphatase subunits PP1β and MYPT1 were used, expression of PP1β and MYPT was markedly reduced and an increase in basal HDAC7 phosphorylation was found (FIG. 11B, right panel). Further, HDAC7 remained phosphorylated up to 8 h after PMA stimulation (FIG. 11B, right panel). This result demonstrated that myosin phosphatase specifically dephosphorylated HDAC7 at later time points after PMA stimulation.

### Example 10: SiRNA-Mediated Knockdown of Myosion Phosphatase Enhances HDAC7 Exclusion from the Nucleus and Delays Nuclear Re-Entry

Next, the effect of siRNA-mediated knockdown of myosin phosphatase on HDAC7 nucleo-cytoplasmic shuttling was examined. To test the role of myosin phosphatase in the nucleo-cytoplasmic shuttling of HDAC7, DO11.10 cells were nucleofected with an HDAC7-GFP expression construct together with specific siRNAs to knockdown PP1β, MYPT1 or both proteins. 24 hours after nucleofection the cells were treated with PMA for 0.5, 2, 4, 8, and 24 hours. The result of this analysis is shown in FIG. 12B. Examination of the nuclear exclusion of HDAC7 in response to PMA and treated with the control siRNA (siCo), showed, as before, a rapid exclusion of HDAC7 from the nucleus following PMA treatment, peaking at 2 hours, with a slow progressive reentry of HDAC7 in the nucleus during the next 22 hours (FIGS. 12A, 12B). Importantly, nuclear export was slightly enhanced when myosin phosphatase subunits were knocked down, and HDAC7 nuclear re-entry was significantly delayed at later time points (FIG. 12B). Thus, in the absence of myosin phosphatase a large percentage of cells showed exclusive cytoplasmic localization even at 24 hours after cell treatment with PMA. These results are in agreement with data showing higher basal phosphorylation and prolonged phosphorylation of HDAC7 after PMA stimulation after knockdown of myosin phosphatase. Similar results were obtained using CD3 crosslinking instead of PMA treatment (data not shown). These observations therefore support the model that HDAC7 is dephosphorylated by myosin phosphatase after stimulation by PMA and TCR activation, leading to its nuclear re-entry. These results demonstrated that myosin phosphatase regulates HDAC7 nucleo-cytoplasmic shuttling.

### Example 11: Suppression of Myosin Phosphatase Vian siRNA Induces Nur77 Expression

HDAC7 is recruited to its target promoters via its specific interaction with the transcription factor MEF2D. (FIGS. 3, 4) A genomic screen of HDAC7 targets has demonstrated that HDAC7 regulates the transcriptional activity of a cassette of genes (FIG. 5). One of the most highly regulated HDAC7 target is the transcription factor Nur77. The experiments performed so far suggested that the level of HDAC7 phosphorylation and the subcellular localization of HDAC7 is under the competing influences of protein kinase D1 and myosin phosphatase. According to this model, the removal of myosin phosphatase should lead to an increase in the cytoplasmic localization of HDAC7, a derepression of Nur77 and an increase in apoptosis.

To test whether myosin phosphatase regulates Nur77 gene expression, an siRNA knockdown experiment was performed (FIG. 13A). Specific siRNAs (as described above) were used to knockdown PP1β, MYPT1 or both proteins in DO11.10 cells (FIG. 13A). This analysis showed that siRNAs directed against PP1β and MYPT1 mRNAs drastically reduced cellular PP1β and MYPT1 proteins. The decrease in protein expression for both proteins as a result of this treatment is shown in FIG. 13A. siRNA-transfected cells were activated via TCR cross-linking (α-CD3 antibody), and the expression of Nur77 was analyzed at 24 h, when Nur77 expression is newly suppressed (FIG. 8B).

As previously reported Nur77 is induced after TCR activation via the crosslinking with anti-CD3 antibody (Parra et al., 2005, J Biol Chem 280(14):13762-70). Surprisingly, in the absence of PP1β or MYPT1, Nur77 was superinduced after TCR engagement (FIG. 13B). The superinduction was higher when both proteins (PP1β and MYPT1) were knocked down (FIG. 13B). Thus, the delay in reentry of HDAC7 is associated with a superinduction of Nur77 expression as shown by Western blot analysis (FIG. 13B). Knockdown of either PP1β or MYPT1, or both together leads to a persistence of Nur77 expression. Importantly, this persistence is abrogated by the expression in the same cells of an HDAC7 mutant in which all three sites of HDAC7 phosphorylation have been mutated (HDAC7ΔP; FIG. 13B, right panel). This result demonstrated that myosin phosphatase is involved in the HDAC7-mediated Nur77 regulation in response to TCR activation. These results further demonstrate that myosin phosphatase mediates the de novo repression of Nur77 expression by dephosphorylating HDAC7 at late times after TCR activation.

To further analyze the specificity of myosin phosphatase in the regulation of Nur77, siRNAs specific for each of the PP1 isoforms, PP1α, PP1β and PP1γ were used (FIG. 13C). PP1β depletion resulted in the superinduction of Nur77 after TCR activation, whereas depletion of PP1α pr PP1γ had no significant effect (FIG. 13D, left panel). Here also, expression of the HDAC7ΔP mutant prevented the superinduction of Nur77 after TCR activation (FIG. 13D, right panel).

### Example 12: Suppression of Myosin Phosphatase Vian siRNA Induces Apoptosis

Nur77 is an orphan nuclear receptor that is rapidly and transiently induced after TCR activation and plays a key role in the induction of negative selection of apoptosis in thymocytes (Liu et al., 1994, Nature 367:281-284; Woronicz et al., 1994, Nature 367:277-281; Calnan et al., 1995, Immunity 3:273-282). In addition, many of the proteins identified in the screen of genomic HDAC7 targets regulated apoptosis in developing thymocytes (FIG. 5).

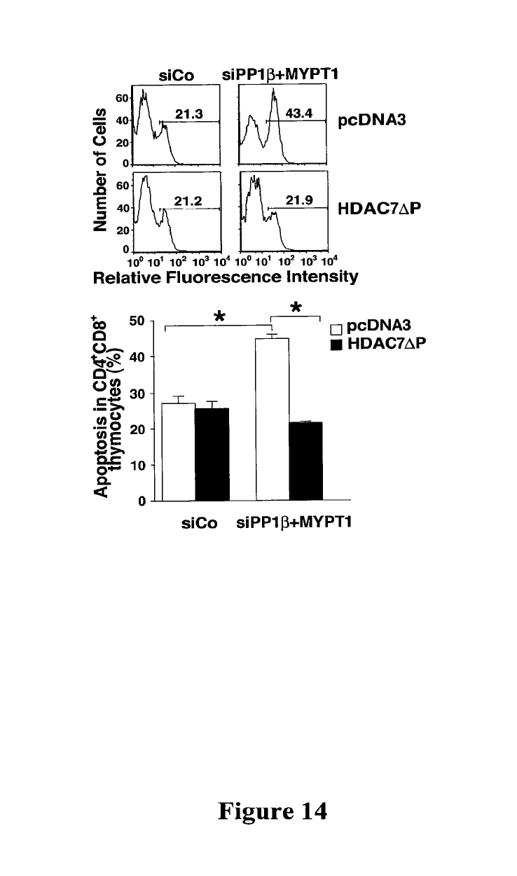

Next, it was analyzed whether the absence of myosin phosphatase also resulted in the increase of apoptosis of T cells. Specific siRNAs against PP1β and MYPT1 were nucleofected into mouse primary thymocytes. Thereafter, thymocytes were stained with CD4, CD8 and Annexin and followed by flow cytometry analysis. It was found that in the presence of an siRNA control (siCo) about 27% of double-positive thymocytes were undergoing apoptosis (FIG. 14). However, the absence of PP1β or MYPT1 resulted in a significant increase in the apoptosis of double-positive thymocytes that was further increased to about 45% when both proteins were knocked-down (FIG. 14). This result demonstrated that myosin phosphatase is involved in the negative selection of thymocytes, i.e., myosin phosphatase mediates the survival of thymocytes via phosphorylation of HDAC7.

### Example 13: An In Vitro Model for Thymic Positive Selection

The experiments described above indicate a critical role of HDAC7 in the control of gene expression for a family of genes that are normally transcriptionally activated during positive and negative selection, including a subset of genes that control apoptosis. Further, the experiments indicated that HDAC7 can control the rate of thymocyte apoptosis.

To address the possible role of HDAC7 in thymocyte differentiation in a more direct manner, an experimental system generated by Kaye and Ellenberger was used (Kaye and Ellenberger, 1992, Cell 71:423-435). It is based on a spontaneously arising thymoma, called DPK, which came from a transgenic mouse expressing a recombinant TCR for pigeon cytochrome C. When these DPK cells are cocultivated with the appropriate antigen presenting cells, e.g., a fibroblastic cell line expressing the class II MHC protein I-E of K and ICAM, in the presence of the appropriate pigeon cytochrome oxidase peptide, the cells undergo a differentiation process similar to positive selection. They lose their CD8 expression and become single positive CD4 T cells.

This system was used to examine the effect of HDAC7. Two different constructs were used, first the HDAC7-VP 16 mutant (see above) and the mutant HDAC7 carrying three mutated phosphorylation sites (see above), the super-repressor.

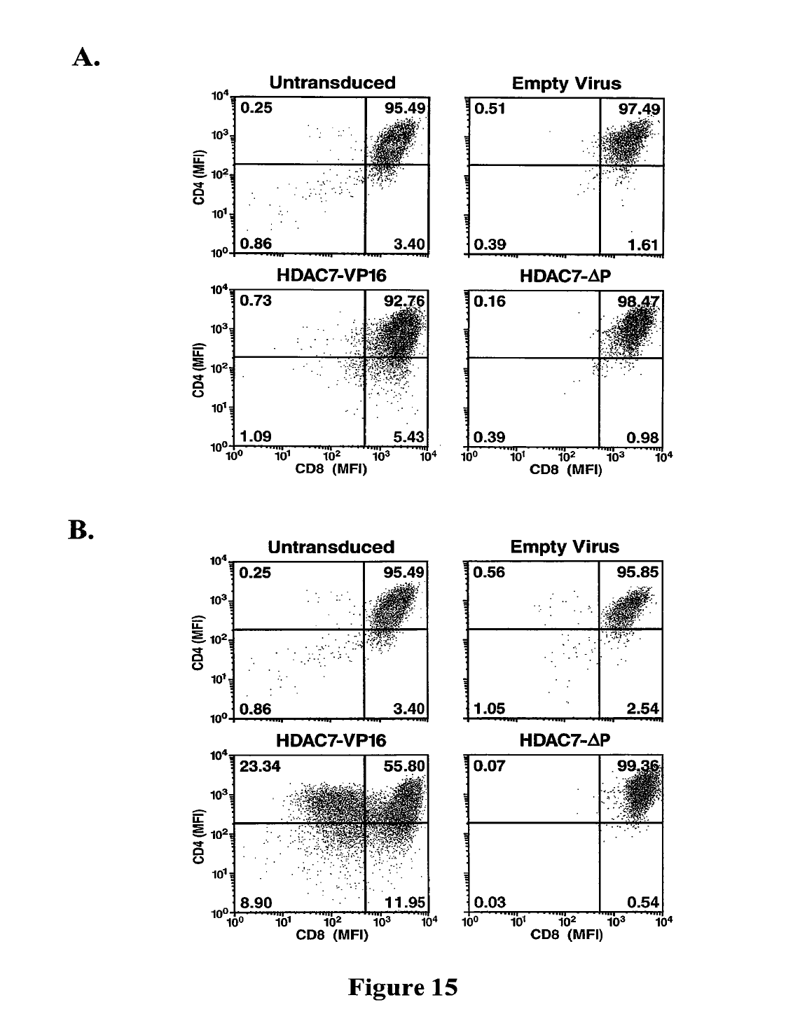

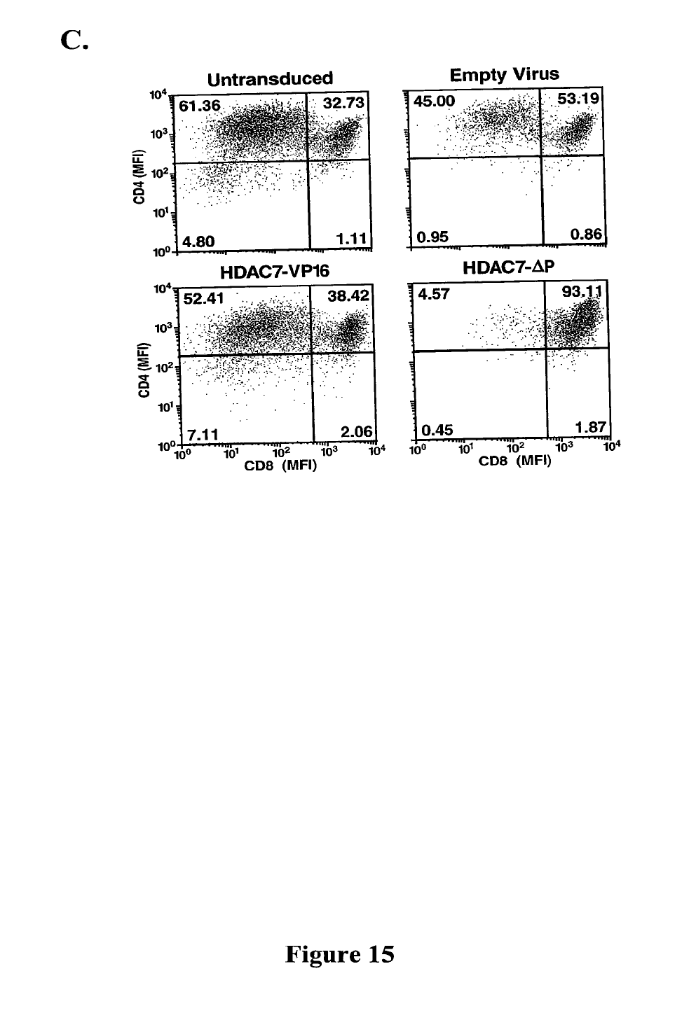

FIG. 15 shows the result of this analysis in form of fax plots where CD8 is on the X axis while CD4 is on the Y axis. The cells start out as double positive, CD4 and CD8 as shown. Under basal conditions, when the cells are cultivated alone, no effect of any of the constructs was observed and the cells were maintained as double positive CD4 and CD8 (FIG. 13A). Remarkably, when the cells were cocultivated with the antigen presenting cells DCEK-ICAM, in the absence of the peptide, HDAC7-VP 16 fusion protein expression was associated with a very significant differentiation of the cells into single positive CD4 T cells (FIG. 13B). When the peptide was added, the cells also became differentiated in CD4 positive T cells, but this effect was largely suppressed by the expression of the HDAC7 superrepressor (FIG. 13C). These results indicate that HDAC7 alone can modulate the rate of differentiation of this cell in in vitro.

### Example 14: Summary And Discussion

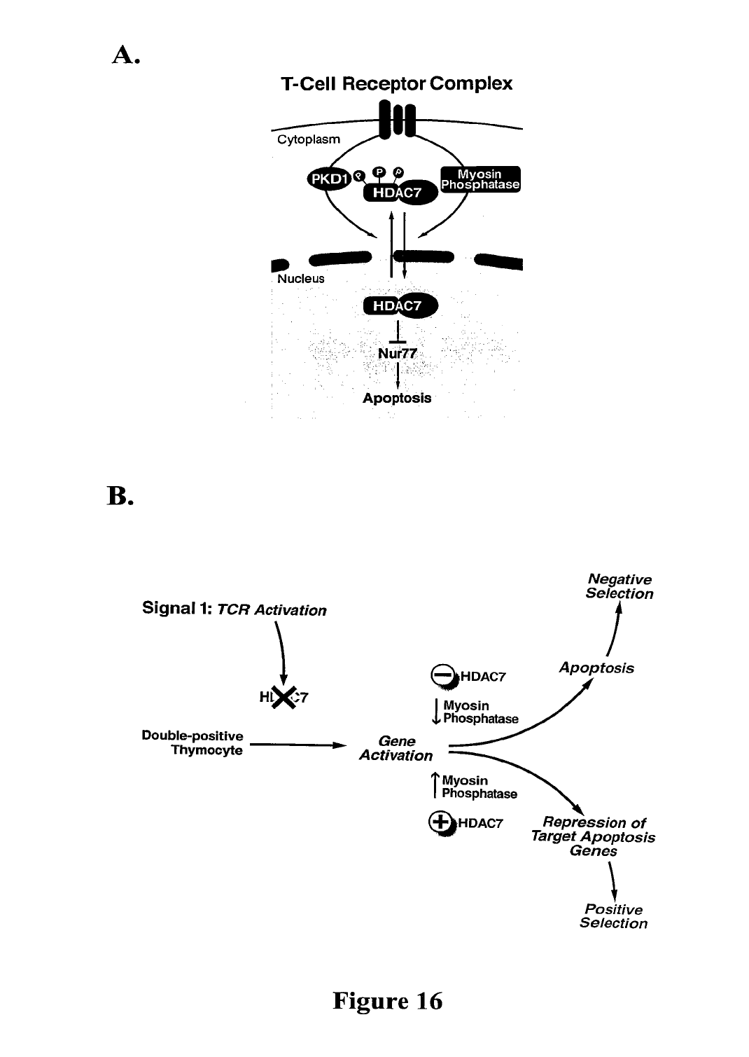

This invention discloses a regulatory mechanism involving reversible acetylation and deacetylation of histone protein catalyzed by histone deacetylase 7 (HDAC7) in T cells. Applicants disclosed herein that the phosphorylation of HDAC7, its nucleo-cytoplasmic shuttling of HDAC7, and its activity as a transcriptional repressor in thymocytes are regulated by a protein kinase (PKD1 phosphorylating the three serine residues in HDAC7) and a phosphatase, myosin phosphatase (FIG. 16A). Further, Applicants identified by immunoprecipitation and mass spectrometry analysis of HDAC7-associated proteins protein phosphatase 10 (PP1β and myosin phosphatase target subunit 1 (MYPT1) as HDAC7-associated proteins. PP1β and MYPT1 form part of a complex named myosin phosphatase that, in addition, includes a subunit called M20. PP1β dephosphorylates HDAC7 in vitro and in vivo. Knockdown of PP1β vian siRNA or its targeting subunit MYPT1 in primary thymocytes lead to the cytoplasmic localization of HDAC7, to derepression of Nur77 expression and to apoptosis induction.

These results indicate that the level of HDAC7 phosphorylation, its subcellular localization (nuclear vs. cytoplasmic) and its role as a transcriptional repressor after T cell receptor (TCR) activation are under the competing influences of PKD and PP1β. Thus, the regulation of PKD 1 and PP1β activities in developing thymocytes plays a critical role in apoptosis and thereby modulate positive vs. negative selection events (FIG. 16B).

The data disclosed here support an important role of HDAC7 in thymocyte differentiation. HDAC7, which is expressed at highest levels in double positive T cells, may represent the effector arm of a differentiation checkpoint that blocks double positive T cells at this stage by suppressing the expression of a set of genes that are critical for both positive and negative selection. During the first 4 h after cross-linking of the TCR (TCR activation), phosphorylation of HDAC7 is enhanced in response to PKD1 activation (Parra et al., 2005, J Biol Chem 280:13762-13770) and, possibly, in response to an inhibition of myosin phosphatase activity. Enhanced phosphorylation of HDAC7 leads to export from the nucleus to the cytoplasm and its functional inactivation as a transcriptional repressor. This leads to the derepression of a set of genes, such as Nur77, that are involved in thymocytes apoptosis. (FIG. 16). Starting 8 h after TCR activation, the activity of myosin phosphatase becomes dominant, leading to the dephosphorylation of HDAC7, its re-entry into the nucleus, and the resilencing of Nur77 and other genes that control apoptosis in developing T cells.

The anti-apoptotic role of myosin phosphatase is particularly intriguing with regard to T-cell development in the thymus. Indeed, a fraction of developing thymocytes responds to TCR activation by further differentiating into single-positive T cells (CD4 or CD8), a process referred to as positive selection. The data presented here support a model in which the level of HDAC7 phosphorylation, controlled by the competing activities of PKD1 and myosin phosphatase, could determine whether the developing T cells undergo positive or negative selection (FIG. 16). Depending on the activity level of myosin phosphatase, two outcomes seem to be possible.

Under some conditions, myosin phosphatase is inactivated. This leads to the persistent transcription and expression of a set of genes controlled by HDAC7 and involved in apoptosis. This persistent expression leads to apoptosis, a process called negative selection during thymocyte development (FIG. 16).

Under some other conditions, myosin phosphatase is activated, dephosphorylates HDAC7 and leads to the repression of the genes that control apoptosis. This allows the developing thymocyte to persist, a process that could lead to positive selection (FIG. 16).

According to this model, a key factor in determining the fate of the developing T cells in response to activation of its T cell receptor is mediated by myosin phosphate (FIG. 14).

Importantly, myosin phosphatase is also expressed in cardiac and skeletal muscle (Fujioka et al., 1998, Genomics 49:59-68; Arimura et al., 2001, J Biol Chem 276:6073-6082). In cardiomyocytes, overexpression of myosin phosphatase subunits results in the abolition of agonist-induced sarcomere organization, a marker of cardiac hypertrophy (Okamoto et al., 2006, Cell Signal 18:1408-1416). Interestingly, an HDAC5 mutant that cannot be phosphorylated (similar to the HDAC7Δ7 described herein and by Dequiedt et al., 2003, Immunity 18:687-698) is refractory to hypertrophic signaling and inhibits cardiomyocyte hypertrophy (Zhang et al., 2002, Cell 110:479-488). We suggest that myosin phosphatase could inhibit cardiac hypertrophy by dephosphorylating HDAC5, resulting in its nuclear localization and the repression of specific target genes. Based on the conservation of the sites of phosphorylation in HDAC4, HDAC5, HDAC7, and HDAC9 and on the presence of myosin phosphatase in the tissues where other class IIa HDACs are expressed (e.g., muscle, heart, and CNS), we further suggest that the mechanism described in this application for HDAC7 also contributes to the regulation of other class IIa HDACs in these other tissues.

1-34. (canceled)

35. An in vitro method for identifying a compound which modulates the dephosphorylation of a histone deacetylase-7 (HDAC7) polypeptide by a myosin phosphatase, the method comprising:
a) contacting a candidate compound with:
i) a purified myosin phosphatase polypeptide comprising an amino acid sequence selected from SEQ NOs: 82, 83, and 84; and
ii) a purified HDAC7 polypeptide comprising the amino acid sequence set forth in SEQ ID NO:81, wherein said HDAC7 polypeptide is recognized by one or more of an antibody raised to the peptide FPLRTV[pSer]EPNLKL (SEQ ID NO:39), an antibody raised to the peptide RPLNRTR[pSer]EPLPPS (SEQ ID NO:40), and an antibody raised to the peptide RPLSRTQ[pSer]SPAAPV (SEQ ID NO:41); and
b) determining the effect of the candidate compound on dephosphorylation of the HDAC7 polypeptide by the myosin phosphatase, wherein said determining comprises determining the phosphorylation status of the HDAC7 polypeptide using an antibody specific for phosphorylated HDAC7,
wherein a candidate compound which modulates the dephosphorylation of the HDAC7 polypeptide, compared to a control in the absence of the candidate compound, is identified as a modulator of dephosphorylation.

36. The method of claim 35, wherein the antibody specific for phosphorylated HDAC7 specifically recognizes a peptide selected from FPLRTV[pSer]EPNLKL (SEQ ID NO:39), RPLNRTR[pSer]EPLPPS (SEQ ID NO:40), and RPLSRTQ[pSer]SPAAPV (SEQ ID NO:41).

37. The method according to claim 36, wherein the antibody specific for phosphorylated HDAC7 specifically recognizes the peptide FPLRTV[pSer]EPNLKL (SEQ ID NO:39).

38. The method according to claim 36, wherein the antibody specific for phosphorylated HDAC7 specifically recognizes the peptide RPLNRTR[pSer]EPLPPS (SEQ ID NO:40).

39. The method according to claim 36, wherein the antibody specific for phosphorylated HDAC7 specifically recognizes the peptide RPLSRTQ[pSer]SPAAPV (SEQ ID NO:41).

40. The method according to claim 35, wherein the antibody specific for phosphorylated HDAC7 I selected from an antibody specifically recognizing a human HDAC7 phosphorylated at amino acid residues 155, 318 or 448.

41. The method according to claim 40, wherein the antibody specific for phosphorylated HDAC7 is an antibody that specifically recognizing a human HDAC7 phosphorylated at amino acid residue 155.

42. The method according to claim 35, wherein the antibody specific for phosphorylated HDAC7 is an antibody that specifically recognizing a human HDAC7 phosphorylated at amino acid residue 318.

43. The method according to claim 35, wherein the antibody specific for phosphorylated HDAC7 is an antibody that specifically recognizing a human HDAC7 phosphorylated at amino acid residue 448.

44. The method according to claim 35, wherein the HDAC7 polypeptide is a naturally occurring HDAC7 polypeptide.

45. The method according to claim 35, wherein the myosin phosphatase polypeptide and the HDAC7 polypeptide comprise:
(i) a naturally occurring HDAC7 polypeptide and a naturally occurring myosin phosphatase;
(ii) a recombinantly produced HDAC7 polypeptide and a recombinantly produced myosin phosphatase; or
(iii) a combination of (i) and (ii).

46. The method according to claim 35, wherein the myosin phosphatase polypeptide comprises the amino acid sequence set forth in SEQ ID NO:82.

47. The method according to claim 35, wherein the myosin phosphatase polypeptide comprises the amino acid sequence set forth in SEQ ID NO:83.

48. The method according to claim 35, wherein the myosin phosphatase polypeptide comprises the amino acid sequence set forth in SEQ ID NO:84.

49. The method of claim 35, further comprising the step of:
(c) determining whether the candidate compound binds to the myosin phosphatase polypeptide.

50. The method of claim 35, further comprising the step of:
(c) determining whether the candidate compound increases the activity of the myosin phosphatase polypeptide.

51. The method of claim 35, further comprising the step of:
(c) determining whether the candidate compound increases binding of the myosin phosphatase polypeptide to the HDAC7 polypeptide.

52. The method of claim 51, wherein step (c) comprises an immunoprecipitation assay.

53. The method of claim 35, further comprising the step of:
(c) determining whether the candidate compound decreases the activity of the myosin phosphatase polypeptide.

54. The method of claim 35, further comprising the step of:
(c) determining whether the candidate compound decreases binding of the myosin phosphatase polypeptide to the HDAC7 polypeptide.


SEQUENCE LISTING (80 sequences)

SEQ ID NO: 1 - RNA, 19 nt, Artificial Sequence
  Synthetic oligonucleotide
ccagaagccaacuaucuuu

SEQ ID NO: 2 - RNA, 19 nt, Artificial Sequence
  Synthetic oligonucleotide
gccaacuaucuuuucuuag

SEQ ID NO: 3 - RNA, 19 nt, Artificial Sequence
  Synthetic oligonucleotide
cggauaugaauuuuuugcu

SEQ ID NO: 4 - DNA, 21 bp, Artificial Sequence
  Synthetic oligonucleotide
ccagaagccaacuaucuuutt

SEQ ID NO: 5 - DNA, 21 bp, Artificial Sequence
  Synthetic oligonucleotide
gccaacuaucuuuucuuagtt

SEQ ID NO: 6 - DNA, 21 bp, Artificial Sequence
  Synthetic oligonucleotide
cggauaugaauuuuuugcutt

SEQ ID NO: 7 - RNA, 21 nt, Artificial Sequence
  Synthetic oligonucleotide
gaacgagacuugcguauguuu

SEQ ID NO: 8 - RNA, 21 nt, Artificial Sequence
  Synthetic oligonucleotide
aagaauaguucgaucaauguu

SEQ ID NO: 9 - RNA, 21 nt, Artificial Sequence
  Synthetic oligonucleotide
cgacaucaauuacgccaauuu

SEQ ID NO: 10 - RNA, 21 nt, Artificial Sequence
  Synthetic oligonucleotide
ucggcaagguguugauauauu

SEQ ID NO: 11 - RNA, 19 nt, Artificial Sequence
  Synthetic oligonucleotide
gaacgugcagcugacagag

SEQ ID NO: 12 - RNA, 19 nt, Artificial Sequence
  Synthetic oligonucleotide
gggcaaguaugggcaguuc

SEQ ID NO: 13 - RNA, 19 nt, Artificial Sequence
  Synthetic oligonucleotide
gguuguagaagauggcuau

SEQ ID NO: 14 - DNA, 21 bp, Artificial Sequence
  Synthetic oligonucleotide
gaacgugcagcugacagagtt

SEQ ID NO: 15 - DNA, 21 bp, Artificial Sequence
  Synthetic oligonucleotide
gggcaaguaugggcaguuctt

SEQ ID NO: 16 - DNA, 21 bp, Artificial Sequence
  Synthetic oligonucleotide
gguuguagaagauggcuautt

SEQ ID NO: 17 - RNA, 19 nt, Artificial Sequence
  Synthetic oligonucleotide
ccgauaaugcuuucuuugg

SEQ ID NO: 18 - RNA, 19 nt, Artificial Sequence
  Synthetic oligonucleotide
gcaagccaagcacuucauu

SEQ ID NO: 19 - RNA, 19 nt, Artificial Sequence
  Synthetic oligonucleotide
cgggcaguacuaugauuug

SEQ ID NO: 20 - DNA, 21 bp, Artificial Sequence
  Synthetic oligonucleotide
ccgauaaugcuuucuuuggtt

SEQ ID NO: 21 - DNA, 21 bp, Artificial Sequence
  Synthetic oligonucleotide
gcaagccaagcacuucauutt

SEQ ID NO: 22 - DNA, 21 bp, Artificial Sequence
  Synthetic oligonucleotide
cgggcaguacuaugauuugtt

SEQ ID NO: 23 - DNA, 21 bp, Artificial Sequence
  Synthetic oligonucleotide
cggauaugaauuuuuugcutt

SEQ ID NO: 24 - DNA, 20 bp, Artificial Sequence
  Synthetic oligonucleotide
ccgauaaugcwucuuuggtt

SEQ ID NO: 25 - RNA, 19 nt, Artificial Sequence
  Synthetic oligonucleotide
augaacgugaauugcucaa

SEQ ID NO: 26 - RNA, 19 nt, Artificial Sequence
  Synthetic oligonucleotide
uaaggcuaugaagagauac

SEQ ID NO: 27 - RNA, 19 nt, Artificial Sequence
  Synthetic oligonucleotide
auguauuggccuguauuag

SEQ ID NO: 28 - RNA, 19 nt, Artificial Sequence
  Synthetic oligonucleotide
uagcgacuaaacacaucaa

SEQ ID NO: 29 - DNA, 21 bp, Artificial Sequence
  Synthetic oligonucleotide
cuuacgcugaguacuucgatt

SEQ ID NO: 30 - DNA, 16 bp, Artificial Sequence
  Synthetic oligonucleotide
tgaccgccatttgcga

SEQ ID NO: 31 - DNA, 17 bp, Artificial Sequence
  Synthetic oligonucleotide
cgtttcccagcaaggca

SEQ ID NO: 32 - DNA, 22 bp, Artificial Sequence
  Synthetic oligonucleotide
tggtctacgacacgttcatgct

SEQ ID NO: 33 - DNA, 20 bp, Artificial Sequence
  Synthetic oligonucleotide
tcagggtgcacgtgtgtgtt

SEQ ID NO: 34 - DNA, 21 bp, Artificial Sequence
  Synthetic oligonucleotide
tggtgtctgctggatttgatg

SEQ ID NO: 35 - DNA, 24 bp, Artificial Sequence
  Synthetic oligonucleotide
atccaaaacatttggcagaaacat

SEQ ID NO: 36 - DNA, 18 bp, Artificial Sequence
  Synthetic oligonucleotide
cctcggaagcatgtgtta

SEQ ID NO: 37 - DNA, 15 bp, Artificial Sequence
  Synthetic oligonucleotide
caccagtgcatgtgc

SEQ ID NO: 38 - DNA, 20 bp, Artificial Sequence
  Synthetic oligonucleotide
ccggccccactgggtggcta

SEQ ID NO: 39 - protein, 13 aa, Artificial Sequence
  Synthetic peptide
FPLRTVSEPNLKL

SEQ ID NO: 40 - protein, 14 aa, Artificial Sequence
  Synthetic peptide
RPLNRTRSEPLPPS

SEQ ID NO: 41 - protein, 14 aa, Artificial Sequence
  Synthetic peptide
RPLSRTQSSPAAPV

SEQ ID NO: 42 - protein, 12 aa, Artificial Sequence
  Synthetic peptide
YLAEFATGNDRK

SEQ ID NO: 43 - protein, 14 aa, Artificial Sequence
  Synthetic peptide
NLLSVAYKNVIGAR

SEQ ID NO: 44 - protein, 12 aa, Artificial Sequence
  Synthetic peptide
MDDREDLVYQAK

SEQ ID NO: 45 - protein, 17 aa, Artificial Sequence
  Synthetic peptide
AASDIAMTELPPTHPIR

SEQ ID NO: 46 - protein, 16 aa, Artificial Sequence
  Synthetic peptide
LAEQAERYDEMVESMK

SEQ ID NO: 47 - protein, 9 aa, Artificial Sequence
  Synthetic peptide
GDREQLLQR

SEQ ID NO: 48 - protein, 16 aa, Artificial Sequence
  Synthetic peptide
LAEQAERYDDMASAMK

SEQ ID NO: 49 - protein, 17 aa, Artificial Sequence
  Synthetic peptide
EAFEISKEHMQPTHPIR

SEQ ID NO: 50 - protein, 19 aa, Artificial Sequence
  Synthetic peptide
NSVVEASEAAYKEAFEISK

SEQ ID NO: 51 - protein, 22 aa, Artificial Sequence
  Synthetic peptide
AVTELNEPLSNEDRNLLSVAYK

SEQ ID NO: 52 - protein, 7 aa, Artificial Sequence
  Synthetic peptide
LAEQAER

SEQ ID NO: 53 - protein, 14 aa, Artificial Sequence
  Synthetic peptide
SVTEQGAELSNEER

SEQ ID NO: 54 - protein, 8 aa, Artificial Sequence
  Synthetic peptide
NLLSVAYK

SEQ ID NO: 55 - protein, 8 aa, Artificial Sequence
  Synthetic peptide
VVSSIEQK

SEQ ID NO: 56 - protein, 15 aa, Artificial Sequence
  Synthetic peptide
VVSSIEQKTEGAEKK

SEQ ID NO: 57 - protein, 12 aa, Artificial Sequence
  Synthetic peptide
FLIPNASQPESK

SEQ ID NO: 58 - protein, 12 aa, Artificial Sequence
  Synthetic peptide
YLAEVAAGDDKK

SEQ ID NO: 59 - protein, 9 aa, Artificial Sequence
  Synthetic peptide
EMQPTHPIR

SEQ ID NO: 60 - protein, 6 aa, Artificial Sequence
  Synthetic peptide
ACSLAK

SEQ ID NO: 61 - protein, 7 aa, Artificial Sequence
  Synthetic peptide
LAEQAER

SEQ ID NO: 62 - protein, 16 aa, Artificial Sequence
  Synthetic peptide
LAEQAERYDDMAAAMK

SEQ ID NO: 63 - protein, 8 aa, Artificial Sequence
  Synthetic peptide
NLLSVAYK

SEQ ID NO: 64 - protein, 8 aa, Artificial Sequence
  Synthetic peptide
VISSIEQK

SEQ ID NO: 65 - protein, 6 aa, Artificial Sequence
  Synthetic peptide
KIEMVR

SEQ ID NO: 66 - protein, 5 aa, Artificial Sequence
  Synthetic peptide
IEMVR

SEQ ID NO: 67 - protein, 10 aa, Artificial Sequence
  Synthetic peptide
YLAEVATGEK

SEQ ID NO: 68 - protein, 11 aa, Artificial Sequence
  Synthetic peptide
YLAEVATGEKR

SEQ ID NO: 69 - protein, 8 aa, Artificial Sequence
  Synthetic peptide
ATWESSEK

SEQ ID NO: 70 - protein, 10 aa, Artificial Sequence
  Synthetic peptide
AYSEAHEISK

SEQ ID NO: 71 - protein, 7 aa, Artificial Sequence
  Synthetic peptide
NVVGGRR

SEQ ID NO: 72 - protein, 5 aa, Artificial Sequence
  Synthetic peptide
GDYFR

SEQ ID NO: 73 - protein, 10 aa, Artificial Sequence
  Synthetic peptide
IYGFYDECKR

SEQ ID NO: 74 - protein, 11 aa, Artificial Sequence
  Synthetic peptide
IYGFYDECKRR

SEQ ID NO: 75 - protein, 16 aa, Artificial Sequence
  Synthetic peptide
YQYGGLNSGRPVTPPR

SEQ ID NO: 76 - protein, 8 aa, Artificial Sequence
  Synthetic peptide
TANPPKKR

SEQ ID NO: 77 - protein, 10 aa, Artificial Sequence
  Synthetic peptide
LAYVTPTIPR

SEQ ID NO: 78 - protein, 7 aa, Artificial Sequence
  Synthetic peptide
TSSSYTR

SEQ ID NO: 79 - protein, 6 aa, Artificial Sequence
  Synthetic peptide
SCSFGR

SEQ ID NO: 80 - protein, 11 aa, Artificial Sequence
  Synthetic peptide
SLPSSTSTAAK